(19) United States
(12) Patent Application Publication
(10) Pub. No.: US 2011/0045053 A1
(43) Pub. Date: Feb. 24, 2011

(54) ISOLATED POPULATION OF LUMINAL STEM CELLS THAT GIVE RISE TO PROSTATE CANCER AND METHODS OF USING SAME

(76) Inventors: Michael M. Shen, New York, NY (US); Corinne T. Abate-Shen, New York, NY (US); Xi Wang, Edison, NJ (US); Marianna Kruithof - De Julio, Edgewater, NJ (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/816,331

(22) Filed: Jun. 15, 2010

Related U.S. Application Data
(60) Provisional application No. 61/234,975, filed on Aug. 18, 2009.

(51) Int. Cl. A61K 9/00; C12N 5/071; G01N 33/567; C12Q 1/04; C12Q 1/68; A61K 35/12; A61P 13/08

(52) U.S. Cl. 424/423; 435/325; 435/354; 435/7.21; 435/34; 435/6; 435/366; 424/93.7

(57) ABSTRACT

The present invention is directed to an isolated population of luminal stem cells obtained from the prostate epithelium that express Nkx3.1. The invention is also directed to methods for diagnosing whether a patient is at risk of developing prostate cancer.

This application claims the benefit of the filing date of U.S. Provisional Patent Application No. 61/234,975, filed Aug. 18, 2009, the contents of which are hereby incorporated by reference.

### GOVERNMENT SUPPORT

The work described herein was supported in whole, or in part, by National Cancer Institute Grant No. U01-CA84294 and by National Institute of Diabetes and Digestive and Kidney Diseases grant No. R01-DK076602. Thus, the United States Government has certain rights to the invention.

All patents, patent applications and publications cited herein are hereby incorporated by reference in their entirety. The disclosures of these publications in their entireties are hereby incorporated by reference into this application in order to more fully describe the state of the art as known to those skilled therein as of the date of the invention described and claimed herein.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Castration-resistant Nkx3.1-expressing cells (CARNs) are luminal cells that are distinct from other prostate cells. CARNs are cells of origin for prostate cancer.

Prostate cancer is one of the most common types of cancer in men, affecting about one in six men in the United States (http://www.mayoclinic.com/health/prostate-cancer/DS00043). Prostate cancer occurs when cells within the prostate grow uncontrollably, creating small tumors.

### SUMMARY OF THE INVENTION

The present invention relates generally to the finding that castration-resistant Nkx3.1-expressing cells (CARNs) are a population of luminal stem cells that are distinct from other prostate cells. This population of stem cells is found, for example, in humans. This population of stem cells is also found in mice. CARNs are cells of origin for prostate cancer.

An aspect of the invention provides for an isolated population of luminal stem cells that can be obtained from the prostate epithelium, and that further express Nkx3.1 in the absence of androgens. In one embodiment, the cells are castration-resistant. In another embodiment, the luminal stem cells further express Androgen receptor (AR), the luminal markers, which include but are not limited to, cytokeratin 8 (CK8), cytokeratin 18 (CK18), or a combination thereof. In a further embodiment, the luminal stem cells fail to express the Ki67 marker. In some embodiments, the luminal stem cells are obtained from the anterior region of the mouse prostate. In other embodiments, the population of luminal stem cells is obtained through a single-cell transplantation assay. In further embodiments, the population of luminal stem cells are human stem cells or mouse stem cells.

An aspect of the invention provides for a population of luminal stem cells from the prostate epithelium isolated by a single-cell transplantation assay, wherein the cells express Nkx3.1 in the absence of androgens. In one embodiment, the cells are castration-resistant. In another embodiment, the luminal stem cells further express Androgen receptor (AR), the luminal markers, which include but are not limited to, cytokeratin 8 (CK8), cytokeratin 18 (CK18), or a combination thereof. In a further embodiment, the luminal stem cells fail to express the Ki67 marker. In some embodiments, the luminal stem cells are obtained from the anterior region of the mouse prostate. In other embodiments, the population of luminal stem cells is obtained through a single-cell transplantation assay. In further embodiments, the population of luminal stem cells are human stem cells or mouse stem cells.

An aspect of the invention provides for an isolated cell of origin for prostate cancer, wherein the cell expresses Nkx3.1 in the absence of androgens. In one embodiment, the cell further expresses cytokeratin 8 (CK8), cytokeratin 18 (CK18), Androgen receptor (AR), or a combination thereof. In another embodiment, the cell fails to express the Ki67 marker.

Another aspect of the invention provides a purified preparation of prostate epithelium luminal stem cells wherein the cells express Nkx3.1 in the absence of androgens and cytokeratin 8 (CK8), cytokeratin 18 (CK18), Androgen receptor (AR), or a combination thereof. In further embodiments, the population of luminal stem cells are human stem cells or mouse stem cells.

A further aspect of the invention provides for a purified preparation of prostate epithelium luminal stem cells wherein the cells express Nkx3.1 in the absence of androgens and cytokeratin 8 (CK8), cytokeratin 18 (CK18), Androgen receptor (AR), or a combination thereof, and do not express Ki67. In further embodiments, the population of luminal stem cells are human stem cells or mouse stem cells.

An aspect of the invention provides for a method for diagnosing whether a patient is at risk of developing prostate cancer. The method comprises (a) obtaining a tissue, a tissue sample, or a cell population; (b) contacting the tissue, the tissue sample, or the cell population with an agent that binds to Nkx3.1; and (c) determining whether the agent has bound to the tissue, the tissue sample, or the cell population, wherein binding indicates the presence of stem cells that express Nkx3.1. In one embodiment, the step of determining is performed using a method selected from the group consisting of RT PCR, in situ hybridization, Northern blotting RNAase protection, or any combination thereof. In another embodiment, the tissue, the tissue sample, or the cell population comprises prostate tissue or cells, bone marrow, peripheral blood, lymph nodes, tumor metastases, or a combination thereof. In a further embodiment, the stem cells are castration-resistant, luminal prostate stem cells. In another embodiment, the castration-resistant, luminal prostate stem cells are human stem cells or mouse stem cells. In some embodiments, the agent is an antibody. In further embodiments, the antibody is a polyclonal antibody or a monoclonal antibody.

A further aspect of the invention provides for a method for diagnosing whether a subject is at risk of developing prostate cancer. The method comprises (a) obtaining a biological sample from a subject; and (b) determining whether or not stem cells that express Nkx3.1 are present in a biological sample from the subject as compared to a non-prostate cancer subject. In one embodiment, the step of determining is performed using a method selected from the group consisting of RT PCR, in situ hybridization, Northern blotting RNAase protection, or any combination thereof. In another embodiment, the tissue, the tissue sample, or the cell population comprises prostate tissue or cells, bone marrow, peripheral blood, lymph nodes, tumor metastases, or a combination thereof. In a further embodiment, the stem cells are castration-resistant, luminal prostate stem cells.

In another aspect, the invention provides for a method for diagnosing prostate cancer stem cells in metastatic cells or metastases. The method comprises (a) obtaining a tissue, a tissue sample, or a cell population; (b) contacting the tissue, the tissue sample, or the cell population with an agent that binds to Nkx3.1; and (c) determining whether the agent has bound to the tissue, the tissue sample, or the cell population, wherein binding indicates the presence of stem cells that express Nkx3.1. In one embodiment, the step of determining is performed using a method selected from the group consisting of RT PCR, in situ hybridization, Northern blotting RNAase protection, or any combination thereof. In another embodiment, the tissue, the tissue sample, or the cell population comprises prostate tissue or cells, bone marrow, peripheral blood, lymph nodes, tumor metastases, or a combination thereof. In a further embodiment, the stem cells are castration-resistant, luminal prostate stem cells. In another embodiment, the castration-resistant, luminal prostate stem cells are human stem cells or mouse stem cells. In some embodiments, the agent is an antibody. In further embodiments, the antibody is a polyclonal antibody or a monoclonal antibody.

An aspect of the invention provides for a method for diagnosing prostate cancer stem cells in metastatic cells or metastases. The method comprises (a) obtaining a biological sample from a subject; and (b) determining whether or not stem cells that express Nkx3.1 are present in a biological sample from the subject as compared to a non-prostate cancer subject. In one embodiment, the step of determining is performed using a method selected from the group consisting of RT PCR, in situ hybridization, Northern blotting RNAase protection, or any combination thereof. In another embodiment, the tissue, the tissue sample, or the cell population comprises prostate tissue or cells, bone marrow, peripheral blood, lymph nodes, tumor metastases, or a combination thereof. In a further embodiment, the stem cells are castration-resistant, luminal prostate stem cells.

An aspect of the invention provides for a diagnostic kit for detecting the presence of Nkx3.1 in a sample, the kit comprising a nucleic acid molecule that specifically hybridizes to or a primer combination that amplifies a Nkx3.1 nucleic acid sequence. In one embodiment, the nucleic acid molecule comprises a nucleic acid primer or nucleic acid probe. In another embodiment, the Nkx3.1 nucleic acid sequence comprises at least about 90% of SEQ ID NO: 20. In a further embodiment, the probe comprises at least 10 consecutive nucleotide bases comprising SEQ ID NO: 20. In some embodiments, the probe comprises a reverse complement of at least 10 consecutive nucleotide bases comprising SEQ ID NO: 20. In one embodiment, the primer comprises a nucleotide sequence that comprises SEQ ID NOS: 9, 11 or a combination thereof. In some embodiments, the sample is from a human or non-human animal. In other embodiments, the sample comprises prostate tissue or cells, bone marrow, peripheral blood, lymph nodes, tumor metastases, or a combination thereof.

An aspect of the invention provides for a diagnostic kit for determining whether a sample from a subject exhibits a presence of prostate cancer or a predisposition to developing prostate cancer, the kit comprising a nucleic acid primer that specifically hybridizes to a luminal prostate cancer biomarker, wherein the primer will prime a polymerase reaction only when a luminal prostate cancer biomarker is present. In some embodiments, the luminal prostate cancer biomarker is Nkx3.1. In one embodiment, the primer comprises a nucleotide sequence that comprises SEQ ID NOS: 9, 11 or a combination thereof. In some embodiments, the sample is from a human or non-human animal. In other embodiments, the sample comprises prostate tissue or cells, bone marrow, peripheral blood, lymph nodes, tumor metastases, or a combination thereof.

An aspect of the inventions provides for methods for reconstituting prostate tissue. The method comprises (a) isolating luminal stem cells expressing Nkx3.1 in the absence of androgens from dissociated prostate cells of a subject; (b) recombining the isolated luminal cells with mesenchymal cells; and (c) performing a graft in an immunodeficient subject. In one embodiment, the graft is a renal graft.

In another aspect, the invention provides methods for identifying a compound that inhibits prostate cancer. The method comprises contacting a population of luminal, prostate epithelium stem cells that express Nkx3.1 in the absence of androgens with a test compound under culture conditions which would cause differentiation of the stem cells into prostate cancer cells, and determining whether the differentiation of prostate cancer cells is inhibited in the presence of the test compound as compared to differentiation of the stem cells in the absence of the test compound. The test compound would be assessed for its ability to block the growth and/or maintenance of the cancer stem cells as compared to the absence of the test compound. In addition, the test compound could be assessed for its ability to induce the differentiation of the cancer stem cells as compared to the absence of the test compound.

An aspect of the invention provides for methods for identifying a compound that inhibits prostate cancer. The method comprises (a) obtaining a population of luminal, prostate epithelium stem cells that express Nkx3.1 in the absence of androgens; (b) contacting the population of luminal, prostate epithelium stem cells with a test compound; and (c) determining whether the a population of luminal, prostate epithelium stem cells that express Nkx3.1 in the absence of androgens fails to form a tumor in a graft.

An aspect of the invention provides methods for identifying a compound that inhibits prostate cancer, the method comprising: (a) obtaining a population of luminal, prostate epithelium stem cells that express Nkx3.1 in the absence of androgens; (b) contacting the population of luminal, prostate epithelium stem cells that express Nkx3.1 in the absence of androgens with a test compound; and (c) determining whether the population of luminal, prostate epithelium stem cells that express Nkx3.1 in the absence of androgens is reprogrammed to an embryonic differentiation pattern in the presence of the test compound as compared to a population of luminal, prostate epithelium stem cells that express Nkx3.1 in the absence of androgens that were not treated with the test compound.

### BRIEF DESCRIPTION OF THE FIGURES

To conform to the requirements for U.S. patent applications, many of the figures presented herein are black and white representations of images originally created in color, such as many of those figures based on immunofluorescence microscopy, yellow fluorescent protein (YFP) labeling, and BrdU (yellow) staining In the below descriptions and the examples, this colored staining is described in terms of its appearance in black and white. For example, Brd-U staining which appeared yellow in the original appears as a dark stain when presented in black and white. The original color versions of FIGS. 1-16 can be viewed in Wang et al., Nature. 2009 Sep. 24; 461(7263):495-500 (including the accompanying Supplementary Information available in the on-line version of the manuscript available on the Nature web site). For the purposes of the U.S., the contents of Wang et al., Nature. 2009 Sep. 24; 461(7263):495-500, including the accompanying “Supplementary Information,” are herein incorporated by reference.

FIG. 1 shows the expression of Nkx3.1 in epithelial cells of the intact and regressed anterior prostate. FIG. 1A is a schematic prostate duct in the intact, regressed, and regenerated states. Most luminal cells undergo apoptosis during regression, whereas most basal cells survive; hence, the process of regeneration primarily produces luminal cells. FIG. 1B is a fluorescence micrograph showing Nkx3.1 expression in all luminal cells of the wild-type intact prostate. FIG. 1C is a fluorescence micrograph showing Nkx3.1 expression is mostly absent in regressed prostate, except for rare castration-resistant Nkx3.1-expressing cells (CARNs, arrows). FIG. 1D is a fluorescence micrograph showing expression of Nkx3.1 in regenerated prostate, showing similarity to FIG. 1B. FIG. 1E is a fluorescence micrograph showing immunostaining for Nkx3.1 and β-catenin shows clustering of CARNs. FIG. 1F-1G are fluorescence micrographs showing CARNs are strictly luminal, as shown by lack of co-staining for Nkx3.1 (arrows) and p63 (FIG. 1F), and by co-localization of Nkx3.1 (arrows) with cytokeratin 18 (CK18) (FIG. 1G). Scale bars correspond to 25 microns.

FIGS. 2A-2E show the bipotentiality and self-renewal of CARNs in vivo. FIG. 2A is a schematic of a strategy for a lineage-marking experiment. FIG. 2B is a schematic of a timeline for the experiment. FIG. 2C is a fluorescence micrograph showing that YFP does not co-localize with p63 in lineage marked cells of a castrated and tamoxifen-induced Nkx3.1^{CreERT2/+}; R26R-YFP/+ anterior prostate. FIG. 2D is a fluorescence micrograph showing that clusters of YFP+ cells in a lineage-marked and regenerated prostate. FIG. 2E is a fluorescence micrograph showing that colocalization of YFP and cytokeratin 5 (CK5) in lineage-marked basal cells (arrows) of a regenerated prostate. Scale bars correspond to 25 microns.

FIGS. 2F-2L show the bipotentiality and self-renewal of CARNs in vivo. FIG. 2F is a schematic of a time-line for self-renewal experiment. FIGS. 2G-I are fluorescence micrographs showing that co-localization of Nkx3.1, YFP, and BrdU immunostaining (arrow) in anterior prostate, shown as an overlay (FIG. 2G) and individual channels (FIGS. 2H-2I); YFP+BrdU+ neighbors are indicated (arrowheads). FIG. 2J is a schematic of a strategy for four round serial regression/regeneration assay of long-term CARNs self-renewal. FIGS. 2K-L are fluorescence micrographs showing that clusters of YFP+ cells in the lineage-marked prostate after four rounds of serial regression/regeneration. Scale bars correspond to 25 microns.

FIGS. 3A-3D show the generation of prostatic ducts in renal grafts by single lineage-marked CARNs. FIG. 3A is a schematic of a strategy for tissue recombinant/renal graft analyses using a single YFP+ cell (or single YFP− cell as a control). FIGS. 3B-3C are microphotographs of hematoxylin-eosin (H&E) staining of prostatic ducts in a graft derived from a single YFP+ cell; note presence of basal cells (arrows) and secretions (FIG. 3C). FIG. 3D is a fluorescence photomicrograph showing epithelial cells in single-YFP+ derived duct express YFP, including p63+ basal cells (arrows). Scale bars in FIG. 3D correspond to 25 microns, in FIGS. 3B-3C to 50 microns.

FIGS. 3E-3J show the generation of prostatic ducts in renal grafts by single lineage-marked CARNs. FIGS. 3E-G are photomicrographs that show the expression of luminal marker CK18 (FIG. 3E), basal marker p63 (FIG. 3F), and neuroendocrine marker synaptophysin (Syn) (FIG. 3G) in ducts from single YFP+ cells. FIGS. 3H-3I show the expression of androgen receptor (AR) (FIG. 3H) and Nkx3.1 (FIG. 3I) that confirm prostate identity of ducts. FIG. 3J is a graph that summarizes single-cell transplantation data. Scale bars in FIGS. 3E-3F, and FIGS. 3H-3I correspond to 25 microns, in FIG. 3G to 50 microns.

FIGS. 4A-4D show Nkx3.1 mutants that display prostate epithelial defects in a serial regeneration assay. FIG. 4A is a schematic of a time-line for analysis of label-retaining cells (LRCs). FIGS. 4B-4D are fluorescent photomicrographs that show the overlap of CARNs with LRCs in a serially regressed prostate, shown as an overlay (FIG. 4B) and individual panels (FIGS. 4C-4D). Arrow in FIGS. 4B-4D indicates a Nkx3.1⁺BrdU⁺ cell; arrowhead in FIG. 4C indicates a CARN that is Brd⁻. Scale bars correspond to 25 microns.

FIGS. 4E-4H show Nkx3.1 mutants that display prostate epithelial defects in a serial regeneration assay. FIG. 4E is a schematic showing a time-line for serial regression/regeneration analyses. FIGS. 4F-4G are fluorescent photomicrographs that show decreased number of LRCs (arrows) in Nkx3.1^{-/-} anterior prostate (FIG. 4G) relative to wild-type controls (FIG. 4F) after serial regeneration. FIG. 4H is a graphs that shows decreased volume of Nkx3.1^{-/-} anterior prostate relative to wild-type and Nkx3.1^{+/-} prostates following serial regeneration, and to intact wild-type and Nkx3.1^{-/-} prostates. Error bars correspond to one standard deviation. Scale bars correspond to 25 microns.

FIG. 5A is a schematic of a timeline for inducible conditional deletion of Pten in CARNs.

FIGS. 5B-5M are photomicrographs showing that the CARNs population contains a cell type of origin for prostate cancer. FIGS. 5B-5E show hematoxylin-eosin (H&E) staining of anterior prostate from control Nkx3.1^{CreERT2/+}; Pten^{+/+} (FIG. 5B and FIG. 5D) and Nkx3.1^{CreERT2/+}; Pten^{flox/flox} mice (FIG. 5C and FIG. 5E), shown at low-power (FIGS. 5B-5C) and high-power (FIGS. 5D-5E). The Nkx3.1^{CreERT2/+}; Pten^{flox/flox} prostate contains high-grade PIN/carcinoma lesions with local invasive epithelium (arrows, FIG. 5E). FIGS. 5F-G show the detection of p63+ basal cells that show loss of basal cells except at the periphery (arrows, FIG. 5G) of PIN/carcinoma lesions. FIGS. 5H-5I show elevated Ki67 immunostaining in PIN/carcinoma lesions. FIGS. 5J-5K show Phospho-Akt immunostaining with cell membrane localization (arrows, FIG. 5K) in PIN/carcinoma lesions. FIGS. 5L-5M show Pten immunostaining is ubiquitous in control Nkx3.1^{CreERT2/+}; Pten^{-/+} prostate epithelium, but is restricted to basal cells and scattered luminal cells in induced Nkx3.1^{CreERT2/+}; pten^{flox/flox} prostate. Scale bars correspond to 100 microns.

FIG. 6 comprises schematics that show possible lineage relationships in the prostate epithelium. FIG. 6A shows that independent stem cells for basal and luminal epithelium may give rise to differentiated cell types through multipotent progenitors (MPP) and transit-amplifying progenitors, with some bipotentiality (dashed arrows). In this model, CARNs would correspond to the luminal stem cells. FIG. 6B shows, alternatively, that stem cells for prostate organogenesis may be basal, but luminal transit-amplifying cells, including CARNs, can acquire stem cell properties during regeneration (solid arrows), thus acting as facultative or “potential” stem cells.

FIG. 7 are photomicrographs that show expression of Nkx3.1 and CD117 (c-kit) in prostatic lobes of androgen-deprived mice. FIG. 7A depicts confocal immunofluorescence detection of Nkx3.1 and p63 in wildtype intact anterior prostate; nuclei are detected by counter-staining with TOPRO3. Representative Nkx3.1+ luminal (lum) and p63+ basal (bas) cells are indicated; arrow indicates a basal cell that co-expresses Nkx3.1 and p63. FIG. 7B shows expression of Nkx3.1 and p63 in wild-type anterior prostate after one round of regression and regeneration. Arrow indicates a basal cell that co-expresses Nkx3.1 and p63. FIGS. 7C-7D show detection of castration-resistant Nkx3.1-expressing cells (CARNs) (arrows) in ventral prostate (VP) (FIG. 7C) and dorsolateral prostate (DLP) (FIG. 7D) of a castrated adult male. FIGS. 7E-7F show detection of CARNs (arrows) in anterior prostate (AP) (FIG. 7E) and dorsolateral prostate (FIG. 7F) in the second-round regressed state, following one round of regeneration and regression. FIG. 7G shows expression of androgen receptor (AR) by a CARN (arrow) in the regressed prostate. FIG. 7H shows that CARNs are growth-quiescent, as shown by lack of co-staining for Nkx3.1 (arrows) and Ki67 (arrowheads) in regressed prostate. FIG. 7I shows detection of CARNs (arrows) in wild-type prostate using an independent Nkx3.1 polyclonal antiserum^{S4}. FIG. 7J shows the absence of Nkx3.1 immunostaining in a Nkx3.1^{-/-} homozygous mutant anterior prostate. FIGS. 7K-7L show Expression of CD117 (c-kit) in the regressed anterior prostate, as detected by two different monoclonal antibodies, ACK2 (FIG. 7K) and ACK45 (FIG. 7L). Note that the rare CD117-positive cells are never luminal. Scale bars correspond to 25 microns.

FIG. 8 shows the generation and analysis of the inducible Nkx3.1^{CreERT2/+} knock-in allele in intact male mice. FIG. 8A is a schematic of the targeting strategy that utilizes the self-excising ACE-Cre/PolII-neo selection cassette from the pACN vector^{S5}, and inserts CreER^{T2}-polyA at the translation start site for Nkx3.1, thereby generating a null allele for Nkx3.1. Excision of the selection cassette by CreloxP recombination occurs by passage through the male germline, and occurs with 100% efficiency. Abbreviations: E, EcoRI, H, HinDIII; X, XbaI. FIG. 8B is a schematic showing that the CreER^{T2} fusion protein is inactive unless transiently activated by tamoxifen. Cre activation can lead to recombination at the R26R-lacZ reporter locus; since this occurs on a cell-by-cell basis, the resulting tissue may be mosaic for lacZ expression. FIGS. 8C-8D are photomicrographs of low-power views of β-galactosidase staining of dorsolateral prostate from intact Nkx3.1^{CreERT2/+}; R26R-lacZ/+ mice, either mock-injected (FIG. 8C) or injected with tamoxifen (FIG. 8D). FIGS. 8E-8F are photomicrographs showing cre-mediated recombination in the anterior prostate of intact Nkx3.1^{CreERT2/+}; R26R-YFP/+ mice following tamoxifen-induction, showing sporadic YFP expression predominantly in luminal cells (FIG. 8E), but also in p63+ basal cells (arrow, FIG. 8F). Scale bars correspond to 100 microns (FIGS. 8C-8D) or 25 microns (FIGS. 8E-8F).

FIGS. 9A-9F are fluorescent photomicrographs that show bipotentiality and self-renewal of CARNs. FIG. 9A shows that co-localization of YFP (arrows) and cytokeratin 14 is not observed in the castrated and tamoxifen-induced Nkx3.1^{CreERT2/+}; R26R-YFP/+ anterior prostate (n=0/131 YFP+ cells). FIG. 9B shows that co-localization of YFP with cytokeratin 5 (CK5) is almost never observed (n=2/93 YFP+ cells); both of the observed YFP+CK5+ cells show atypical basal morphology (inset). FIG. 9C shows the co-localization of YFP with cytokeratin 18 (CK18) (n=123/123 YFP+ cells). FIG. 9D shows the co-expression of YFP with androgen receptor (AR) (n=94/94). FIG. 9E shows the overlap of Cre and YFP expression (arrows) in castrated and tamoxifen induced Nkx3.1^{CreERT2/+}; R26R-YFP/+ anterior prostate at four days following tamoxifen administration in the regressed state. FIG. 9F shows the persistence of lineage-marked cells (arrows) in the androgen-deprived and tamoxifen-induced Nkx3.1^{CreERT2/+}; R26R-YFP/+ prostate epithelium, using direct visualization of YFP. Mice were castrated at two months of age and tamoxifen induced after four weeks of regression, then maintained in the androgen-deprived state until analysis at ten months of age. Scale bars correspond to 25 microns.

FIGS. 9G-9L are fluorescent photomicrographs that show bipotentiality and self-renewal of CARNs. Co-localization of YFP and p63 in a lineage-marked basal cells (arrow) of a castrated, tamoxifen-induced, and regenerated Nkx3.1^{CreERT2/+}; R26R-YFP/+ anterior prostate, shown as an overlay (FIG. 9G) and as individual channels (FIGS. 9H-9I). FIGS. 9J-9L show co-localization of β-galactosidase and cytokeratin 14 in a lineage-marked basal cell (arrow) of a castrated, tamoxifen induced, and regenerated Nkx3.1^{CreERT2/+}; R26R-lacZ/+ anterior prostate, shown as an overlay (FIG. 9J) and as individual channels (FIGS. 9K-9L). Scale bars correspond to 25 microns.

FIG. 9M is a schematic showing the strategy for analysis of self-renewal. Nkx3.1^{CreERT2/+}; R26R-YFP/+ male mice are castrated, tamoxifen-induced, and regenerated, with BrdU administered during the first three days of regeneration, followed by removal of androgens and prostate regression. Triple-positive Nkx3.1+YFP+BrdU+ cells would correspond to cells that were CARNs in both the first and second regressions, and had undergone proliferation, consistent with self renewal.

FIG. 10A is a schematic of a strategy for analysis of CARNs potential in tissue recombinant/renal grafts generated using approximately 160 lineage-marked YFP+ cells.

FIGS. 10B-10G are fluorescent photomicrographs that show the generation of prostate tissue by dissociated CARNs in tissue recombinants. FIGS. 10B-10D show the contribution of lineage marked YFP+ cells in renal grafts grown from dissociated cells of castrated and tamoxifen induced Nkx3.1^{CreERT2/+}; R26R-YFP/+ mice combined with rat urogenital mesenchyme. The percentage of lineage-marked basal cells varies significantly in these grafts; compare their relative absence in FIG. 10B (arrowheads indicate non-marked basal cells) to their abundance in FIG. 10D. FIGS. 10E-10G show higher power view of duct in FIG. 10D shows numerous YFP+p63+basal cells (arrows); insets show colocalization of YFP and p63. Scale bars correspond to 25 microns.

FIG. 11 are photomicrographs that show the generation of prostatic ducts in renal grafts by single lineage marked CARNs. FIGS. 11A-11F show bright-field (FIGS. 11A-11B) and epifluorescence (FIGS. 11D-11E) views of dissociated prostate cells from lineage-marked Nkx3.1^{CreERT2/+}; R26R-YFP/+ prostate tissue (FIG. 11A, FIG. 11D) and single YFP+ cells isolated by mouth-pipetting (FIG. 11B, FIG. 11E). Dark-field (FIG. 11C) and epifluorescence (FIG. 11F) views of renal grafts after growth for 2.5 months. FIGS. 11G-11H show expression of E-cadherin (FIG. 11G) and cytokeratin 5 (CK5) (FIG. 11H) in ducts from single YFP+ cells. Scale bars correspond to 25 microns (FIGS. 11A-11B, FIGS. 11D-11E, FIGS. 11G-11H) or 1 mm (FIG. 11C, FIG. 11F).

FIG. 12 comprises fluorescent photomicrographs showing distinct morphology of mouse and rat nuclei in renal grafts. High-power images of DAPI-stained ducts from tissue recombinants/renal grafts show that mouse nuclei (FIG. 12A) contain multiple punctate staining regions, while rat nuclei (FIG. 12B) generally do not^{S6}. Note that the epithelium from the single-cell graft in FIG. 12A contains mouse epithelium (epi) and rat stroma (str), while both the epithelium and stroma in FIG. 12B are of rat origin.

FIG. 13A is a schematic of a timeline for serial regression/regeneration strategy.

FIGS. 13B-13I are photomicrographs showing histopathological analysis of wild-type and Nkx3.1^{-/-} mutant prostates after three rounds of serial regression/regeneration. FIGS. 13B-13E show immunostaining for the basal cell marker p63 in wild-type (FIG. 13B, FIG. 13D; arrow, FIG. 13D) and Nkx3.1^{-/-} serially-regenerated mutant prostate (FIG. 13C, FIG. 13E; arrow, FIG. 13E); note that an area of the Nkx3.1^{-/-} prostate in (FIG. 13C) lacks epithelial cells (arrows). FIGS. 13F-13G show that a-smooth muscle actin (SMA) marks stroma in wild-type (FIG. 13F) and Nkx3.1^{-/-} (FIG. 13G) serially-regenerated prostate; no difference in staining pattern is observed. FIGS. 13H-13I show that Nkx3.1 immunostaining is detected in wild-type (FIG. 13H), but not Nkx3.1^{-/-} (FIG. 13I) mutant prostate after three rounds of serial regeneration. Scale bars correspond to 50 microns.

FIG. 14A is a schematic of a timeline for serial regression/regeneration strategy.

FIGS. 14B-14K are photomicrographs showing histopathological analysis of wild-type and Nkx3.1^{-/-} mutant prostates after five rounds of serial regression/regeneration. FIGS. 14B-14E are photomicrographs of hematoxylin-eosin staining of wild-type (FIG. 14B, FIG. 14D) and Nkx3.1^{-/-} mutant (FIG. 14C, FIG. 14E) anterior prostate after serial regeneration, shown at low (FIGS. 14B-14C) and high-power (FIGS. 14D-14E). Note the relative absence of epithelial hyperplasia typically found in intact Nkx3.1^{-/-} mutants, as well as an increase in basal cells in limited regions (arrow, FIG. 14E). FIGS. 14F-14G show an increased number of p63+ basal cells (arrow, FIG. 14G) in regions of serially regenerated Nkx3.1^{-/-} mutant prostates. FIGS. 14H-14I show a similar proliferative index in serially regenerated wild-type (FIG. 14H) and Nkx3.1^{-/-} mutant (FIG. 14I) anterior prostates, as determined by Ki67 immunostaining (arrows). FIGS. 14J-14K show α-smooth muscle actin (SMA) marks stroma in wild-type and Nkx3.1^{-/-} serially-regenerated anterior prostate. No difference in staining patterns is observed, in contrast with the significantly attenuated SMA staining in intact Nkx3.1^{-/-} mutant prostate at similar ages^{S2}. Scale bars correspond to 100 microns (FIGS. 14B-14G) or 50 microns (FIGS. 14H-14K).

FIGS. 14L-14S are photomicrographs showing histopathological analysis of wild-type and Nkx3.1^{-/-} mutant prostates after five rounds of serial regression/regeneration. FIGS. 14L-14M show androgen receptor (AR) immunostaining shows an identical pattern in wild-type (FIG. 14L) and Nkx3.1−/− (FIG. 14M) serially-regenerated anterior prostate. Note that AR immunoreactivity is modestly increased in Nkx3.1−/− mutant prostate epithelium, as has been previously reported for intact mice^{S7}. FIGS. 14N-14O show that Nkx3.1 immunostaining can be detected in wild-type (FIG. 14N), but not Nkx3.1−/− (FIG. 14O) mutant prostate after five rounds of serial regeneration. FIGS. 14P-14Q show that synaptophysin (Syn) immunoreactivity detects rare neuroendocrine cells in serially regenerated wild-type (FIG. 14P) and Nkx3.1−/− mutant (FIG. 14Q) anterior prostates. FIGS. 14R-14S depicts Hematoxylin-eosin (H&E) staining of dorsolateral (DLP) prostate that shows that the phenotype of the serially regenerated Nkx3.1−/− mutant DLP (FIG. 14R) resembles that of the wild-type DLP control (FIG. 14S). Scale bars correspond to 150 microns (FIGS. 14L-14S).

FIG. 15 are schematics of a model for Nkx3.1 function in prostate stem cell maintenance. Note that for simplicity, stem cells and multipotent progenitors (MPP) are not depicted as either luminal or basal. FIG. 15A shows that in intact Nkx3.1 mutant mice, stem cell self-renewal may be impaired, leading to increased differentiation of lineage-restricted transit amplifying cells and consequent epithelial hyperplasia. FIG. 15B shows that depletion of stem cells and transit amplifying cells through serial regression/regeneration would lead to reversion of the hyperplasia phenotype due to androgen dependent apoptosis of luminal cells during regression, accompanied by potential accumulation of androgen-independent basal cells.

FIG. 16 is a summary of single-cell transplantation data showing the formation of mouse ducts formed or rat ducts formed.

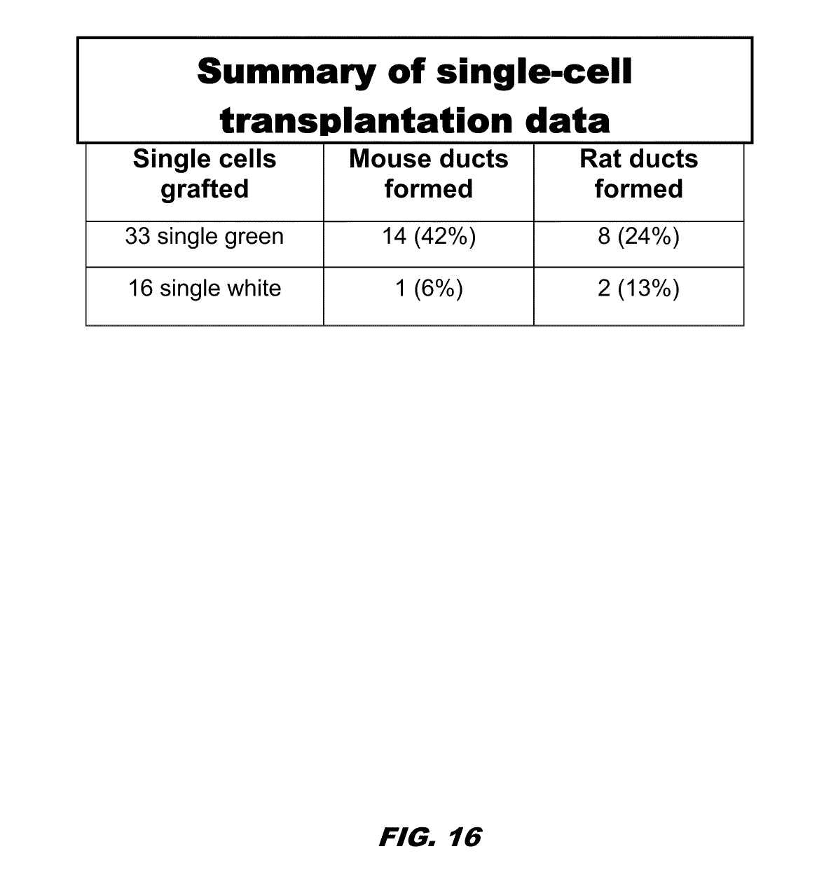

FIG. 17 comprises fluorescent photomicrographs showing reprogramming of adult stem cells to embryonic progenitors. Grafts grown from single-cell transplants of CARNs for two months contain ducts with morphologies ranging from adult-like (left panel, arrow) to neonatal (center, right panels), with luminal cells expressing the basal marker p63 (center, right panels, arrows).

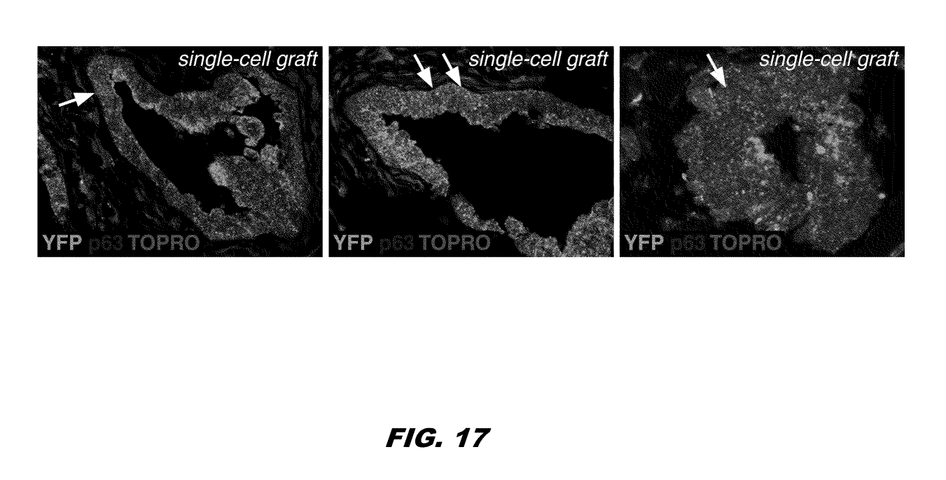

FIGS. 18A-C are photomicrographs showing the identification of prostate tumor-initiating cells by lineage-marked CARNs in renal grafts. Renal grafts were generated using YFP lineage-marked CARNs obtained from castrated and tamoxifen-induced Nkx3.1^{CreERT2/+}; Pten^{flox/flox}; Kras^{LSL/+}; R26R-YFP/+ prostate, and grown for one month. (A) Ductal morphology of graft containing multi-layered epithelium and nuclear atypia characteristic of PIN. (B) Region containing YFP⁺ cells with mesenchymal morphology, suggestive of an epithelial-mesenchymal transition. (C) Region of PIN with membrane-associated phospho-Akt, consistent with Pten inactivation. (D) Time line for generation of YFP⁺ lineage-marked CARNs for renal grafting. FIG. 18D is a schematic of a timeline for generation of YFP⁺ lineage-marked CARNs for renal grafting.

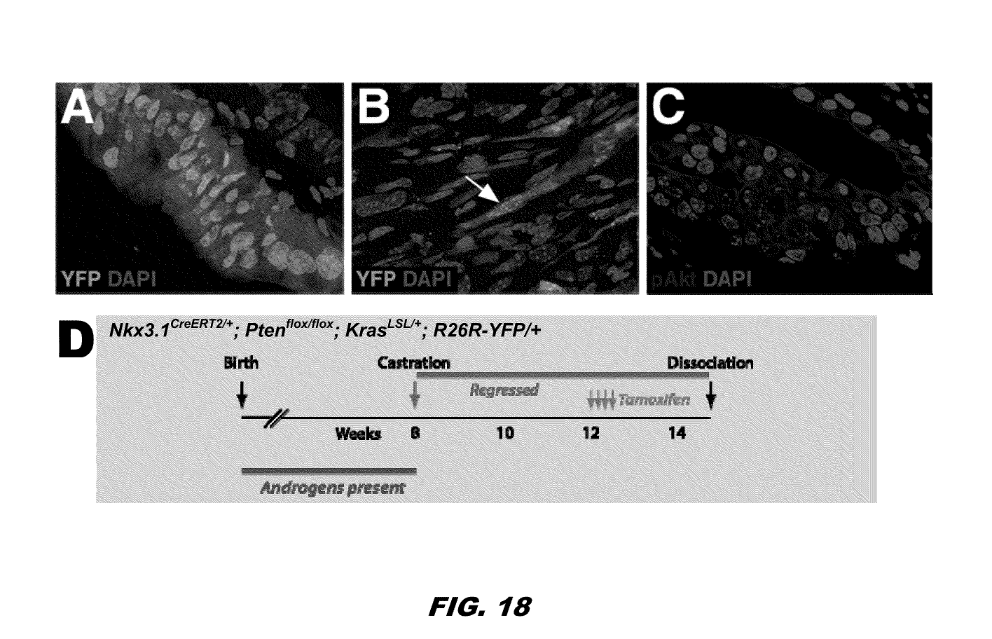

FIG. 19 are photomicrographs of the comparison of mouse (top panel) and human CARNs (bottom panel). Bottom panel shows detection of NKX3.1 expression in regressed human prostate tissue, obtained from a patient who had undergone neoadjuvant androgen-ablation prior to radical prostatectomy. Similar to mouse CARNs, human CARNs are extremely rare, and are usually but not always clustered.

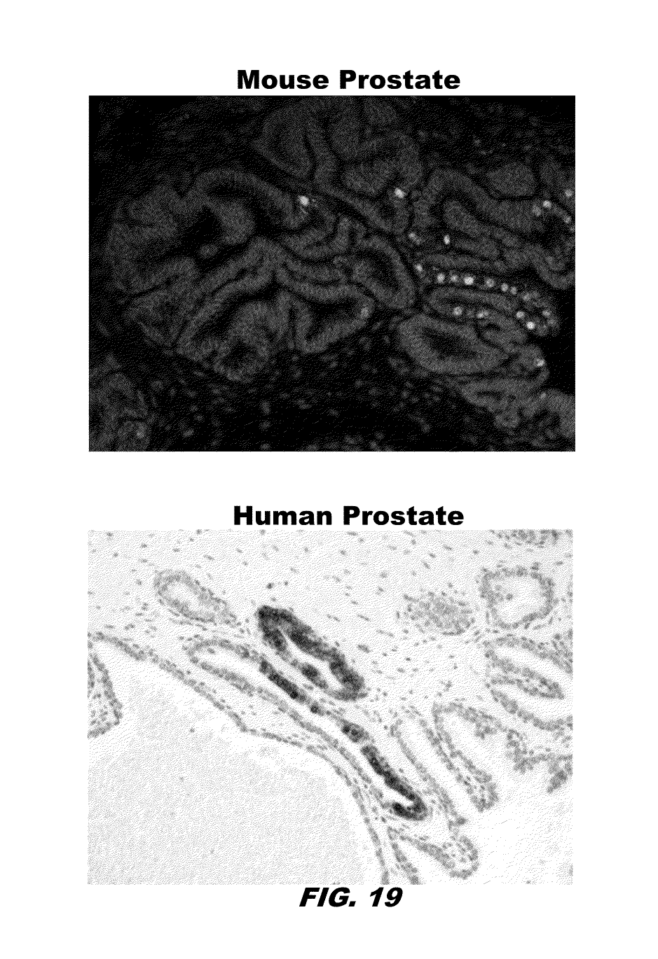

### DETAILED DESCRIPTION OF THE INVENTION

Prostate cancer is among the most diagnosed and prevalent types of cancer in men over the age of 55. In 2005, around 232,100 American men were diagnosed with this cancer [U.S. Cancer Molecular Diagnostics Markets N39E-55 Frost & Sullivan].

Prostate cancer is currently screened by performing a Prostate-Specific Antigen (PSA) blood test together with a Digital Rectal Exam (DRE), and by next performing a biopsy if either of the initial tests reveals abnormal results. The current market for PSA tests alone in the US is valued at about $200 million with the worldwide market at approximately $500 million. The US Prostate Cancer Molecular Diagnostics (CMD) market is estimated to be $5.1 million in 2008 and will grow to a $76.1 million market by 2014 at a CAGR of 62.9 percent [U.S. Cancer Molecular Diagnostics Markets N39E-55 Frost & Sullivan].

Most common treatments for prostate cancer are androgen ablation, radiation therapy and radical prostateclomy. Common prescribed drug for prostate cancer is dominated by hormone therapy, both anti-androgens and LHRH [European Prostate Cancer Therapeutics Markets M173-52 Frost & Sullivan].

The lineage relationship between normal progenitor cells and cell type(s) of origin for cancer has been poorly understood. The invention herein relates to a rare population of stem cells: castration-resistant Nkx3.1-expressing cells (CARNs), for the prostate epithelium in mice. This population of stem cells is an efficient target for oncogenic transformation, and thus is a cell of origin for prostate cancer.

A rare population of stem cells (CARNs, for castration-resistant Nkx3.1-expressing cells) have been identified for the prostate epithelium in mice. This has involved the use of genetically-engineered mice (Nkx3.1-CreERT2) that can be used to label the population of cells, This population has been demonstrated to have stem cell properties by 1) genetic lineage-marking in vivo, and 2) single-cell transplantation in renal grafts. This population of stem cells is an efficient target for oncogenic transformation, and thus is a cell of origin for prostate cancer. Genetic lineage-marking can be used to demonstrate the stem cell properties of CARNs. In one embodiment, CARNs can be human CARNs or mouse CARNs.

In one embodiment, the relationship between CARNs and potential prostate tumor-initiating cells (e.g., cancer stem cells) arising from oncogenic transformation of CARNs can be determined. The identification of CARNs and transformed CARNs (potential cancer stem cells) in human prostate tumor tissue can have prognostic significance. Molecular characterization of CARNs and transformed CARNs can also provide therapeutic insights into prostate cancer diagnosis and treatment.

CARNs Expressing Nkx3.1

CARNs are cells of origin for prostate cancer, distinct from all the other prostate stem cells that have been reported. Thus, they can be more physiologically relevant, especially given that human prostate cancer has a luminal phenotype. This stem cell population and the genetically-engineered animal model developed in this technology may also be used as novel disease model for prostate cancer research.

A Nkx3.1 gene, also known as NK-3 transcription factor, encodes the NK3 homeobox 1 protein. The homeodomain-containing transcription factor NKX3.1 is a putative prostate tumor suppressor that is expressed in a largely prostate-specific and androgen-regulated manner. Loss of NKX3.1 protein expression is a common finding in human prostate carcinomas and prostatic intraepithelial neoplasia. In the context of the invention, the Nkx3.1 gene also encompasses its variants, analogs and fragments thereof, including alleles thereof (e.g., germline mutations) which are related to susceptibility to prostate cancer.

The Nkx3.1 gene locus can comprise all Nkx3.1 sequences or products in a cell or organism, including Nkx3.1 coding sequences, Nkx3.1 non-coding sequences (e.g., introns), Nkx3.1 regulatory sequences controlling transcription and/or translation (e.g., promoter, enhancer, terminator).

SEQ ID NO: 19 is the human wild type amino acid sequence corresponding to the NK-3 transcription factor (residues 1-234) having GenBank Accession No. NP_{—}006158:

| | |
|---|---|
| 1 | MLRVPEPRPG EAKAEGAAPP TPSKPLTSFL IQDILRDGAQ RQGGRTSSQR QRDPEPEPEP |
| 61 | EPEGGRSRAG AQNDQLSTGP RAAPEEAETL AETEPERHLG SYLLDSENTS GALPRLPQTP |
| 121 | KQPQKRSRAA FSHTQVIELE RKFSHQKYLS APERAHLAKN LKLTETQVKI WFQNRRYKTK |
| 181 | RKQLSSELGD LEKHSSLPAL KEEAFSRASL VSVYNSYPYY PYLYCVGSWS PAFW |

SEQ ID NO: 20 is the human wild type nucleic acid sequence corresponding to the NK-3 transcription factor (bps 1-3281) having GenBank Accession No. NM_{—}006167:

| | |
|---|---|
| 1 | gcggtgcggg ccgggcgggt gcattcaggc caaggcgggg ccgccgggat gctcagggtt |
| 61 | ccggagccgc ggcccgggga ggcgaaagcg gagggggccg cgccgccgac cccgtccaag |
| 121 | ccgctcacgt ccttcctcat ccaggacatc ctgcgggacg gcgcgcagcg gcaaggcggc |
| 181 | cgcacgagca gccagagaca gcgcgacccg gagccggagc cagagccaga gccagaggga |
| 241 | ggacgcagcc gcgccggggc gcagaacgac cagctgagca ccgggccccg cgccgcgccg |
| 301 | gaggaggccg agacgctggc agagaccgag ccagaaaggc acttggggtc ttatctgttg |
| 361 | gactctgaaa acacttcagg cgcccttcca aggcttcccc aaacccctaa gcagccgcag |
| 421 | aagcgctccc gagctgcctt ctcccacact caggtgatcg agttggagag gaagttcagc |
| 481 | catcagaagt acctgtcggc ccctgaacgg gcccacctgg ccaagaacct caagctcacg |
| 541 | gagacccaag tgaagatatg gttccagaac agacgctata agactaagcg aaagcagctc |
| 601 | tcctcggagc tgggagactt ggagaagcac tcctctttgc cggccctgaa agaggaggcc |
| 661 | ttctcccggg cctccctggt ctccgtgtat aacagctatc cttactaccc atacctgtac |
| 721 | tgcgtgggca gctggagccc agctttttgg taatgccagc tcaggtgaca accattatga |
| 781 | tcaaaaactg ccttccccag ggtgtctcta tgaaaagcac aaggggccaa ggtcagggag |
| 841 | caagaggtgt gcacaccaaa gctattggag atttgcgtgg aaatctcaga ttcttcactg |
| 901 | gtgagacaat gaaacaacag agacagtgaa agttttaata cctaagtcat tcctccagtg |
| 961 | catactgtag gtcatttttt ttgcttctgg ctacctgttt gaaggggaga gagggaaaat |
| 1021 | caagtggtat tttccagcac tttgtatgat tttggatgag ttgtacaccc aaggattctg |
| 1081 | ttctgcaact ccatcctcct gtgtcactga atatcaactc tgaaagagca aacctaacag |
| 1141 | gagaaaggac aaccaggatg aggatgtcac caactgaatt aaacttaagt ccagaagcct |
| 1201 | cctgttggcc ttggaatatg gccaaggctc tctctgtccc tgtaaaagag aggggcaaat |
| 1261 | agagagtctc caagagaacg ccctcatgct cagcacatat ttgcatggga gggggagatg |
| 1321 | ggtgggagga gatgaaaata tcagcttttc ttattccttt ttattccttt taaaatggta |
| 1381 | tgccaactta agtatttaca gggtggccca aatagaacaa gatgcactcg ctgtgatttt |
| 1441 | aagacaagct gtataaacag aactccactg caagaggggg ggccgggcca ggagaatctc |
| 1501 | cgcttgtcca agacaggggc ctaaggaggg tctccacact gctgctaggg gctgttgcat |
| 1561 | ttttttatta gtagaaagtg gaaaggcctc ttctcaactt ttttcccttg ggctggagaa |
| 1621 | tttagaatca gaagtttcct ggagttttca ggctatcata tatactgtat cctgaaaggc |
| 1681 | aacataattc ttccttccct ccttttaaaa ttttgtgttc ctttttgcag caattactca |
| 1741 | ctaaagggct tcattttagt ccagattttt agtctggctg cacctaactt atgcctcgct |
| 1801 | tatttagccc gagatctggt cttttttttt tttttttttt ttttttttcc gtctccccaa |
| 1861 | agctttatct gtcttgactt tttaaaaaag tttgggggca gattctgaat tggctaaaag |
| 1921 | acatgcattt ttaaaactag caactcttat ttctttcctt taaaaataca tagcattaaa |
| 1981 | tcccaaatcc tatttaaaga cctgacagct tgagaaggtc actactgcat ttataggacc |
| 2041 | ttctggtggt tctgctgtta cgtttgaagt ctgacaatcc ttgagaatct ttgcatgcag |
| 2101 | aggaggtaag aggtattgga ttttcacaga ggaagaacac agcgcagaat gaagggccag |
| 2161 | gcttactgag ctgtccagtg gagggctcat gggtgggaca tggaaaagaa ggcagcctag |
| 2221 | gccctgggga gcccagtcca ctgagcaagc aagggactga gtgagccttt tgcaggaaaa |
| 2281 | ggctaagaaa aaggaaaacc attctaaaac acaacaagaa actgtccaaa tgctttggga |
| 2341 | actgtgttta ttgcctataa tgggtcccca aaatgggtaa cctagacttc agagagaatg |
| 2401 | agcagagagc aaaggagaaa tctggctgtc cttccatttt cattctgtta tctcaggtga |
| 2461 | gctggtagag gggagacatt agaaaaaaat gaaacaacaa aacaattact aatgaggtac |
| 2521 | gctgaggcct gggagtctct tgactccact acttaattcc gtttagtgag aaacctttca |
| 2581 | attttctttt attagaaggg ccagcttact gttggtggca aaattgccaa cataagttaa |
| 2641 | tagaaagttg gccaatttca ccccattttc tgtggtttgg gctccacatt gcaatgttca |
| 2701 | atgccacgtg ctgctgacac cgaccggagt actagccagc acaaaaggca gggtagcctg |
| 2761 | aattgctttc tgctctttac atttctttta aaataagcat ttagtgctca gtccctactg |
| 2821 | agtactcttt ctctcccctc ctctgaattt aattctttca acttgcaatt tgcaaggatt |
| 2881 | acacatttca ctgtgatgta tattgtgttg caaaaaaaaa aaaaaagtgt ctttgtttaa |
| 2941 | aattacttgg tttgtgaatc catcttgctt tttccccatt ggaactagtc attaacccat |
| 3001 | ctctgaactg gtagaaaaac atctgaagag ctagtctatc agcatctgac aggtgaattg |
| 3061 | gatggttctc agaaccattt cacccagaca gcctgtttct atcctgttta ataaattagt |
| 3121 | ttgggttctc tacatgcata acaaaccctg ctccaatctg tcacataaaa gtctgtgact |
| 3181 | tgaagtttag tcagcacccc caccaaactt tatttttcta tgtgtttttt gcaacatatg |
| 3241 | agtgttttga aaataaagta cccatgtctt tattagattt a |

SEQ ID NO: 21 is the mouse wild type amino acid sequence corresponding to the NK-3 transcription factor (residues 1-237) having GenBank Accession No. NP_{—}035051:

| | |
|---|---|
| 1 | MLRVAEPREP RVEAGGRSPW AAPPTQSKRL TSFLIQDILR DRAERHGGHS GNPQHSPDPR |
| 61 | RDSAPEPDKA GGRGVAPEDP PSIRHSPAET PTEPESDAHF ETYLLDCEHN PGDLASAPQV |
| 121 | TKQPQKRSRA AFSHTQVIEL ERKFSHQKYL SAPERAHLAK NLKLTETQVK IWFQNRRYKT |
| 181 | KRKQLSEDLG VLEKNSPLSL PALKDDSLPS TSLVSVYTSY PYYPYLYCLG SWHPSFW |

SEQ ID NO: 22 is the mouse wild type nucleic acid sequence corresponding to the NK-3 transcription factor (bps 1-3137) having GenBank Accession No. NM_{—}010921:

| | |
|---|---|
| 1 | cagcggagga gggatgctta gggtagcgga gccccgagag ccacgggtgg aggcgggtgg |
| 61 | ccgcagtcct tgggcagcgc cacccacgca gtccaagcgg ctcacctcct tcctcatcca |
| 121 | ggacatcctg cgggaccgcg cggagcggca cgggggacac tcaggcaatc cgcagcactc |
| 181 | gccggaccct aggagggact ccgctccaga gcccgacaaa gcagggggtc gcggcgtggc |
| 241 | tccggaggac ccaccaagta tccggcatag ccccgcggag acaccgactg aacccgagtc |
| 301 | tgatgcacat tttgagactt atcttttgga ctgtgaacat aatccagggg acttagcaag |
| 361 | tgccccccag gtcaccaagc agccacagaa gcgctcccgg gccgccttct ctcacactca |
| 421 | ggtgattgag ttggagagga agttcagcca tcagaagtac ctgtctgccc ctgagagggc |
| 481 | tcacctggcc aagaacctca aactcaccga aacccaagtc aaaatatggt tccagaacag |
| 541 | acgctataag accaagcgaa agcagctgtc ggaagacctg ggagtcttgg agaagaactc |
| 601 | accattgtct ttgccagccc tgaaagatga cagcctgccc agtacctcct tggtctccgt |
| 661 | gtatactagc tatccctact acccctacct gtactgtctg ggcagctggc atccatcttt |
| 721 | ctggtagcaa cagttctgct gataactgca tatatcagaa actgtcttcc ctgggggtct |
| 781 | ctgggaaaaa gccacagtgg ctgatgtcaa ggagtcggaa gcaagaaatg tacagatgca |
| 841 | aactgttggg ggttcccagg gaacactcca attcttctct ggtgggcagg tgaaagatct |
| 901 | ggggcagtga ctatttgaag atgtagtccc agttccaagt gtcctgtagg tgactgctgc |
| 961 | ccgctcccct gtttagagag agccaggcaa gtttgagtct tgtttccaga acttagaatg |
| 1021 | gctacagatg caatggctat atcgttccta ggaattctgt tgtggcaact ccatcctctc |
| 1081 | ccatgactga gtatcatgga agggccaagc aaggggaggg atggcccctt agatgaggat |
| 1141 | ttcctcttga gttaaacttg ggtctcctgt tggccttgga ctctggccag agtttctctc |
| 1201 | tgcccttgta aaaatgaggg taaacggaag atttgaagaa tgggggtggg agggggtgac |
| 1261 | ccttagtgat tgaggcaggt tggcatagaa cgtgaagggg gtggaaggtg gaattttcaa |
| 1321 | cttacccttt tcacagtgat gtgccagtcc aaggatttac agggcagcct gagtgtggca |
| 1381 | gggtacacac gcggaggggg ttaagacaag ctgtgtaaac agtttgcctg caggggtggg |
| 1441 | ctgcaaaaca gacttcctct ccaagacatg ggcctatggg gtttccacac gccaggtgag |
| 1501 | cggcttccat caggacttta tattagcagg gaagaaaagg actctcctgc ccttcctgct |
| 1561 | tagactcagg agggcctgca gcttccctgg cctgaaaatt cacactttgt gatgacagag |
| 1621 | gtgccattag ttttaaacaa aggaccttcc aaccctcctt tttatttctt gttctttcct |
| 1681 | acaggacctc tccgttcatg cttttcatct gtgcctgtct tatttcgtgt aagatttgct |
| 1741 | cccacctcct cccaaggttt acctttcttg gtttaacaac ttaactagct acagacagat |
| 1801 | tatgacatac ttttaaaaga aaaaaatgag caactcatct cttcccttta aaaatatgac |
| 1861 | atactttaga gagagaaaaa aaacctagca actcctctct ctctttcctt taaaaatact |
| 1921 | cagcattaaa tcccaagccc tatttaaaga catgagagct gaagatgaca ggttctttcg |
| 1981 | cagggctgtg tgctagctct gtgttacagt tcaaaggctg atgcccattg gggtcctggg |
| 2041 | gatccctgta ggcagaggtg gtttaggggt gcttgttttt catgtgtaag gaagaacagg |
| 2101 | acaaaaggaa gggctggcac tgctgaggga tctgggggag atggaggggt cctgctgtca |
| 2161 | gcccaagcta accagcaagg agaggtaaac tgtcttcaaa atacaacctc accgaggctt |
| 2221 | ccatccacag atgtgtgtgt gtgtgtgtgc gcgcgcgcat gcacgcgcaa atgctttgga |
| 2281 | gaactatgct taggaactac cctaaagtgg taggagtgaa aaagagaatt ggttgacctt |
| 2341 | cccttttcca tcctgatatc tcaggtaacc tgccgaatgg gagataaagg aggtagagca |
| 2401 | cacttataac aaatgagact ttctggggcc aggaaatccc tttatcccac cactcagtgc |
| 2461 | tatacagtga gaaatctttt aatttcctcc ttttaaatgg gccaatttac tgttggtggc |
| 2521 | aaaactgcca aaggaagttg atagaaagct ggcggcttgc accctgttct ttgacagccc |
| 2581 | gggtccccat gttaaagtat tgttccgtgt cccttgttgc tgaccactgt ggtagcgtta |
| 2641 | gaccatacca aagccggagg agtccagttc tcttctttct atccgtcttt tggctctgag |
| 2701 | ttccctatca gtactttctc tcctctcact ctgactagaa tctttttcag cgtgcacttt |
| 2761 | cactacgttt gaactattat agtgtgcagt gtgtggcaaa gtcagaaagg gaaaggcttg |
| 2821 | tgtttaaaat tatgttgggg aagtctgact caattttgct ttctccatac tggaactacc |
| 2881 | cattcaccca cacctgaact ggtagaaacg tggcctgttg tctccagcct gtgaatggga |
| 2941 | aaggtatcgc tgtgggtatc acctgggtga catatctctg tcctgtttcg tcaatcagtt |
| 3001 | agcaaacccc ggctctaaca gaaaagtctg tcacttggca tttagtatag tactccactc |
| 3061 | aaaactttat ttttctacgg gttttttttt tttttgcaac ctatgagtgt tttaaaataa |
| 3121 | agcctctatg tctttat |

As used herein, a “Nkx3.1 molecule” means a nucleic acid which encodes a polypeptide that exhibits homeobox 1 transcription factor activity, or a polypeptide or peptidomimetic that exhibits homeobox 1 transcription factor activity. For example, a Nkx3.1 molecule can include the human Nkx3.1 protein (e.g., having the amino acid sequence shown in SEQ ID NO: 19), or a variant thereof, such as a fragment thereof, that exhibits homeobox 1 transcription factor activity. The nucleic acid can be any type of nucleic acid, including genomic DNA, complementary DNA (cDNA), synthetic or semi-synthetic DNA, as well as any form of corresponding RNA. For example, a Nkx3.1 molecule can comprise a recombinant nucleic acid encoding human Nkx3.1 protein. In one embodiment, a Nkx3.1 molecule can comprise a non-naturally occurring nucleic acid created artificially (such as by assembling, cutting, ligating or amplifying sequences). A Nkx3.1 molecule can be double-stranded. A Nkx3.1 molecule can be single-stranded. The Nkx3.1 molecule of the invention can be obtained from various sources and can be produced according to various techniques known in the art. In one embodiment, Nkx3.1 is expressed by CARNs.

For example, a nucleic acid that is a Nkx3.1 molecule can be obtained by screening DNA libraries, or by amplification from a natural source (such as prostate tissue, or prostate cancer cells). The Nkx3.1 molecule of the invention can be produced via recombinant DNA technology and such recombinant nucleic acids can be prepared by conventional techniques, including chemical synthesis, genetic engineering, enzymatic techniques, or a combination thereof. Non-limiting examples of a Nkx3.1 molecule that is a nucleic acid, is the nucleic acid having the nucleotide sequence shown in SEQ ID NO: 20. Another example of a Nkx3.1 molecule is a fragment of a nucleic acid having the sequence shown in SEQ ID NO: 20, wherein the fragment is exhibits homeobox 1 transcription factor activity. In one embodiment, a Nkx3.1 nucleic acid sequence is expressed by luminal prostate stem cells, CARNs.

The nucleic acids used to practice the invention, whether RNA, RNAi, antisense nucleic acid, cDNA, genomic DNA, vectors, viruses or hybrids thereof, can be produced or isolated from a variety of sources, genetically engineered, amplified, and/or expressed/generated recombinantly. Recombinant polypeptides generated from these nucleic acids can be individually isolated or cloned and tested for a desired activity. Any recombinant expression system can be used, including bacterial, mammalian, yeast, insect or plant cell expression systems. Alternatively, these nucleic acids can be synthesized in vitro by well-known chemical synthesis techniques, as described in, e.g., Adams, J. Am. Chem. Soc. 105:661, 1983; Belousov, Nucleic Acids Res. 25:3440-3444, 1997; Frenkel, Free Radic. Biol. Med. 19:373-380, 1995; Blommers, Biochemistry 33:7886-7896, 1994; Narang, Meth. Enzymol. 68:90, 1979; Brown Meth. Enzymol. 68:109, 1979; Beaucage, Tetra. Lett. 22:1859, 1981; U.S. Pat. No. 4,458,066, all of which are incorporated by reference in their entireties. Techniques for the manipulation of nucleic acids, such as, subcloning, labeling probes (for example, random-primer labeling using Klenow polymerase, nick translation, amplification), sequencing, and hybridization are well described in the scientific and patent literature, see, e.g., Sambrook, ed., MOLECULAR CLONING: A LABORATORY MANUAL (3^{rd} Ed.), Vols. 1-3, Cold Spring Harbor Laboratory, 2001; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, Part I. Theory and Nucleic Acid Preparation, Tijssen, ed. Elsevier, N.Y., 1993.

Nucleic acids or polypeptides can be analyzed and quantified by any of a number of general means well known to those of skill in the art. These include, for example, analytical biochemical methods such as radiography, electrophoresis, NMR, spectrophotometry, capillary electrophoresis, thin layer chromatography (TLC), high performance liquid chromatography (HPLC), and hyperdiffusion chromatography; various immunological methods, such as immuno-electrophoresis, Southern analysis, Northern analysis, dot-blot analysis, fluid or gel precipitation reactions, immunodiffusion, quadrature radioimmunoassay (RIAs), enzyme-linked immunosorbent assays (ELISAs), immuno-fluorescent assays, gel electrophoresis (e.g., SDS-PAGE), nucleic acid or target or signal amplification methods, radiolabeling, scintillation counting, and affinity chromatography.

According to this invention, a Nkx3.1 molecule encompasses orthologs of human Nkx3.1. For example, a Nkx3.1 molecule encompasses the orthologs in mouse, rat, non-human primates, canines, goat, rabbit, porcine, feline, and horses. In other words, a Nkx3.1 molecule can comprise a nucleic acid sequence homologous to the human nucleic acid that encodes a human Nkx3.1, wherein the nucleic acid is found in a different species and wherein that homolog encodes a protein with a homeobox transcription factor function similar to Nkx3.1 molecule.

The variants can comprise, for instance, naturally-occurring variants due to allelic variations between individuals (e.g., polymorphisms), mutated alleles related to prostate cancer, or alternative splicing forms. In one embodiment, a Nkx3.1 molecule is a nucleic acid variant of the nucleic acid having the sequence shown in SEQ ID NO: 20, wherein the variant has a nucleotide sequence identity to SEQ ID NO: 20 of at least about 65%, at least about 75%, at least about 85%, at least about 90%, at least about 91%, at least about 92%, at least about 93%, at least about 94%, at least about 95%, at least about 96%, at least about 97%, at least about 98%, or at least about 99%.

In one embodiment, a Nkx3.1 molecule encompasses any portion of at least about 8 consecutive nucleotides of SEQ ID NO: 20. In one embodiment, the fragment can comprise at least about 15 nucleotides, at least about 20 nucleotides, or at least about 30 nucleotides of SEQ ID NO: 20. Fragments include all possible nucleotide lengths between about 8 and 100 nucleotides, for example, lengths between about 15 and 100, or between about 20 and 100.

The invention further provides for nucleic acids that are complementary to a nucleic acid encoding a Nkx3.1 protein. Such complementary nucleic acids can comprise nucleic acid sequences, which hybridize to a nucleic acid sequence encoding a Nkx3.1 protein under stringent hybridization conditions. Non-limiting examples of stringent hybridization conditions include temperatures above 30° C., above 35° C., in excess of 42° C., and/or salinity of less than about 500 mM, or less than 200 mM. Hybridization conditions can be adjusted by the skilled artisan via modifying the temperature, salinity and/or the concentration of other reagents such as SDS or SSC.

In one embodiment, a Nkx3.1 molecule comprises a protein or polypeptide encoded by SEQ ID NO: 20 or 22. In another embodiment, the polypeptide can be modified, such as by glycosylations and/or acetylations and/or chemical reaction or coupling, and can contain one or several non-natural or synthetic amino acids. An example of a Nkx3.1 molecule is the polypeptide having the amino acid sequence shown in SEQ ID NO: 19 or 21. In one embodiment, a Nkx3.1 polypeptide is expressed by luminal prostate stems cells, CARNs

In another embodiment, a Nkx3.1 molecule can be a fragment of a Nkx3.1 protein. For example, the Nkx3.1 molecule can encompass any portion of at least about 8 consecutive amino acids of SEQ ID NO: 19 or 21. The fragment can comprise at least about 10 amino acids, a least about 20 amino acids, at least about 30 amino acids, at least about 40 amino acids, a least about 50 amino acids, at least about 60 amino acids, or at least about 75 amino acids of SEQ ID NO: 19 or 21. Fragments include all possible amino acid lengths between about 8 and 100 about amino acids, for example, lengths between about 10 and 100 amino acids, between about 15 and 100 amino acids, between about 20 and 100 amino acids, between about 35 and 100 amino acids, between about 40 and 100 amino acids, between about 50 and 100 amino acids, between about 70 and 100 amino acids, between about 75 and 100 amino acids, or between about 80 and 100 amino acids.

In certain embodiments, the Nkx3.1 molecule of the invention includes variants of the human Nkx3.1 protein (having the amino acid sequence shown in SEQ ID NO: 19 and 21, respectively). Such variants can include those having at least from about 46% to about 50% identity to SEQ ID NO: 19 or 21, or having at least from about 50.1% to about 55% identity to SEQ ID NO: 19 or 21, or having at least from about 55.1% to about 60% identity to SEQ ID NO: 19 or 21, or having from at least about 60.1% to about 65% identity to SEQ ID NO: 19 or 21, or having from about 65.1% to about 70% identity to SEQ ID NO: 19 or 21, or having at least from about 70.1% to about 75% identity to SEQ ID NO: 19 or 21, or having at least from about 75.1% to about 80% identity to SEQ ID NO: 19 or 21, or having at least from about 80.1% to about 85% identity to SEQ ID NO: 19 or 21, or having at least from about 85.1% to about 90% identity to SEQ ID NO: 19 or 21, or having at least from about 90.1% to about 95% identity to SEQ ID NO: 19 or 21, or having at least from about 95.1% to about 97% identity to SEQ ID NO: 19 or 21, or having at least from about 97.1% to about 99% identity to SEQ ID NO: 19 or 21. In another embodiment, the Nkx3.1 molecule of the invention encompasses a peptidomimetic which exhibits homeobox 1 transcription factor activity.

A peptidomimetic is a small protein-like chain designed to mimic a peptide that can arise from modification of an existing peptide in order to protect that molecule from enzyme degradation and increase its stability, and/or alter the molecule's properties (for example modifications that change the molecule's stability or biological activity). These modifications involve changes to the peptide that can not occur naturally (such as altered backbones and the incorporation of non-natural amino acids). Drug-like compounds may be able to be developed from existing peptides. A peptidomimetic can be a peptide, partial peptide or non-peptide molecule that mimics the tertiary binding structure or activity of a selected native peptide or protein functional domain (e.g., binding motif or active site). These peptide mimetics include recombinantly or chemically modified peptides.

In one embodiment, a Nkx3.1 molecule comprising SEQ ID NO: 19, SEQ ID NO: 21, variants of each, or fragments thereof, can be modified to produce peptide mimetics by replacement of one or more naturally occurring side chains of the 20 genetically encoded amino acids (or D amino acids) with other side chains. This can occur, for instance, with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, ifuryl, imidazolidinyl imidazolyl, imidazolinyl, isothiazolyl, isoxazolyl, morpholinyl (e.g. morpholino), oxazolyl, piperazinyl (e.g. 1-piperazinyl), piperidyl (e.g. 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g. 1-pyrrolidinyl), pyrrolinyl, pyrrolyl, thiadiazolyl, thiazolyl, thienyl, thiomorpholinyl (e.g. thiomorpholino), and triazolyl. These heterocyclic groups can be substituted or unsubstituted. Where a group is substituted, the substituent can be alkyl, alkoxy, halogen, oxygen, or substituted or unsubstituted phenyl. Peptidomimetics may also have amino acid residues that have been chemically modified by phosphorylation, sulfonation, biotinylation, or the addition or removal of other moieties. For example, peptidomimetics can be designed and directed to amino acid sequences encoded by a Nkx3.1 molecule comprising SEQ ID NO: 19 or 21.

A variety of techniques are available for constructing peptide mimetics with the same or similar desired biological activity as the corresponding native but with more favorable activity than the peptide with respect to solubility, stability, and/or susceptibility to hydrolysis or proteolysis (see, e.g., Morgan & Gainor, Ann. Rep. Med. Chem. 24,243-252, 1989). Certain peptidomimetic compounds are based upon the amino acid sequence of the peptides of the invention. Peptidomimetic compounds can be synthetic compounds having a three-dimensional structure (i.e. a peptide motif) based upon the three-dimensional structure of a selected peptide. The peptide motif provides the peptidomimetic compound with the desired biological activity, wherein the binding activity of the mimetic compound is not substantially reduced, and is often the same as or greater than the activity of the native peptide on which the mimetic is modeled. Peptidomimetic compounds can have additional characteristics that enhance their therapeutic application, such as increased cell permeability, greater affinity and/or avidity and prolonged biological half-life. Peptidomimetic design strategies are readily available in the art (see, e.g., Ripka & Rich, Curr. Op. Chem. Biol. 2, 441452, 1998; Hrubyet al., Curr. Op. Chem. Biol. 1, 114119, 1997; Hruby & Balse, Curr. Med. Chem. 9, 945-970,-2000).

Methods Isolating or Purifying Stem Cells

The present invention provides methods for separating, enriching, isolating or purifying stem cells from a tissue or mixed population of cells. The methods comprise obtaining a mixed population of cells, contacting the population of cells with an agent that binds to Nkx3.1, and separating the subpopulation of cells that are bound by the agent from the subpopulation of cells that are not bound by the agent, wherein the subpopulation of cells that are bound by the agent is enriched for Nkx3.1-positive stem cells (e.g., CARNs).

The methods for separating, enriching, isolating or purifying stem cells from a mixed population of cells according to the invention may be combined with other methods for separating, enriching, isolating or purifying stem or progenitor cells that are known in the art. For example, the methods described herein may be performed in conjunction with techniques that use other stem cell markers. For example, an additional selection step may be performed either before, after, or simultaneously with the Nkx3.1 selection step, in which a second agent, such as an antibody, that binds to a second stem cell marker is used. The second stem cell marker may be any stem cell marker known in the art. In one embodiment, the second stem cell marker is cytokeratin 18 (CK18). In another embodiment, the second stem cell marker is Androgen receptor (AR). The mixed population of cells can be any source of cells from which to obtain Nkx3.1-positive stem cells (e.g., CARNs), including but not limited to a tissue biopsy from a subject, a dissociated cell suspension derived from a tissue biopsy, or a population of cells that have been grown in culture.

The agent used can be any agent that binds to Nkx3.1, as described above. The term “Agent” includes, but is not limited to, small molecule drugs, peptides, proteins, peptidomimetic molecules, and antibodies. It also includes any Nkx3.1 binding molecule that is labeled with a detectable moiety, such as a histological stain, an enzyme substrate, a fluorescent moiety, a magnetic moiety or a radio-labeled moiety. Such “labeled” agents are particularly useful for embodiments involving isolation or purification of Nkx3.1-positive cells, or detection of Nkx3.1-positive cells. In some embodiments, the agent is an antibody that binds to Nkx3.1.

There are many cell separation techniques known in the art, and any such technique may be used. For example magnetic cell separation techniques can be used if the agent is labeled with an iron-containing moiety. Cells may also be passed over a solid support that has been conjugated to an agent that binds to Nkx3.1, such that the Nkx3.1-positive cells will be selectively retained on the solid support. Cells may also be separated by density gradient methods, particularly if the agent selected significantly increases the density of the Nkx3.1-positive cells to which it binds. For example, the agent can be a fluorescently labeled antibody against Nkx3.1, and the Nkx3.1-positive stem cells are separated from the other cells using fluorescence activated cell sorting (FACs).

Methods for Detecting Stem Cells

The present invention also provides methods for detecting stem cells in a tissue, a tissue sample, or a cell population. In one embodiment, the method comprises obtaining a tissue, a tissue sample, or a cell population, contacting the tissue, tissue sample or cell population with an agent that binds to Nkx3.1, and determining whether the agent has bound to the tissue, tissue sample or cell population. Thus, the binding indicates the presence of stem cells expressing Nkx3.1 (e.g., CARNs) and the absence of binding indicates the absence of such stem cells. The agent used can be any agent that binds to Nkx3.1, as described herein.

Diagnosis

The invention provides diagnosis methods based on monitoring a gene encoding Nkx3.1 or stem cells (e.g., CARNs) that express Nkx3.1. As used herein, the term “diagnosis” includes the detection, typing, monitoring, dosing, comparison, at various stages, including early, pre-symptomatic stages, and late stages, of prostate cancer in adults. Diagnosis can include the assessment of a predisposition or risk of development, the prognosis, or the characterization of a subject to define most appropriate treatment (pharmacogenetics). In one embodiment, the invention provides diagnostic methods to determine whether an individual is at risk of developing prostate cancer. A method of detecting the presence of or a predisposition prostate cancer in a subject is provided. In one embodiment, the subject is a human or a non-human animal. Non-limiting examples of non-human animals include primates (such as monkeys), rodents, (such as mice, rats and rabbits), ovine species (such as sheep and goats), bovine species (such as cows), porcine species, equine species, feline species and canine species. In a particular embodiment, the subject is a human. The method can comprise detecting in a sample from the subject the presence of a Nkx3.1 molecule or the presence of Nkx3.1-positive stem cells, such as CARNs. In one embodiment, the detecting comprises detecting the expression of a Nkx3.1 molecule. In some embodiments, the detecting comprises detecting in the sample the presence of an mRNA encoding a Nkx3.1 molecule. In other embodiments, the detecting comprises detecting the presence of luminal prostate stem cells that express Nkx3.1, such as CARNs. The presence of Nkx3.1 or CARNs is indicative of the presence or predisposition to prostate cancer. The presence of a gene encoding a Nkx3.1 molecule in the sample is detected through genotyping a sample, for example via gene sequencing, selective hybridization, amplification, gene expression analysis, or a combination thereof. In one embodiment, the sample can comprise prostate tissue.

The presence of Nkx3.1 can be determined at the DNA, RNA or polypeptide level. The detection can also be determined by performing an oligonucleotide ligation assay, a confirmation based assay, a hybridization assay, a sequencing assay, an allele-specific amplification assay, a microsequencing assay, a melting curve analysis, a denaturing high performance liquid chromatography (DHPLC) assay (for example, see Jones et al, (2000) Hum Genet., 106(6):663-8), or a combination thereof. In some embodiments, the detection is performed by sequencing all or part of a Nkx3.1 gene or by selective hybridization or amplification of all or part of a Nkx3.1 gene.

In another embodiment, the method can comprise detecting the presence of Nkx3.1 RNA expression, for example in a population of prostate stem cells. RNA expression includes the presence of an RNA sequence, the presence of an RNA splicing or processing, or the presence of a quantity of RNA. These can be detected by various techniques known in the art, including by sequencing all or part of the Nkx3.1 RNA, or by selective hybridization or selective amplification of all or part of the RNA. In a further embodiment, the method can comprise detecting the presence of a Nkx3.1 polypeptide expression. Polypeptide expression includes the presence of a Nkx3.1 polypeptide sequence, or the presence of an elevated quantity Nkx3.1 polypeptide as compared to a non-prostate cancer sample. These can be detected by various techniques known in the art, including by sequencing and/or binding to specific ligands (such as antibodies).

Various techniques known in the art can be used to detect or quantify DNA expression, RNA expression, or nucleic acid sequences, which include, but are not limited to, hybridization, sequencing, amplification, and/or binding to specific ligands (such as antibodies). Other suitable methods include allele-specific oligonucleotide (ASO), oligonucleotide ligation, allele-specific amplification, Southern blot (for DNAs), Northern blot (for RNAs), single-stranded conformation analysis (SSCA), PFGE, fluorescent in situ hybridization (FISH), gel migration, clamped denaturing gel electrophoresis, denaturing HLPC, melting curve analysis, heteroduplex analysis, RNase protection, chemical or enzymatic mismatch cleavage, ELISA, radio-immunoassays (RIA) and immuno-enzymatic assays (IEMA). Some other approaches are based on specific hybridization between nucleic acids from the subject and a probe specific for wild type gene or RNA. The probe can be in suspension or immobilized on a substrate. The probe can be labeled to facilitate detection of hybrids. Some of these approaches are suited for assessing a polypeptide sequence or expression level, such as Northern blot, ELISA and RIA. These latter require the use of a ligand-specific for the polypeptide, for example, the use of a specific antibody.

Sequencing. Sequencing can be carried out using techniques well known in the art, using automatic sequencers. The sequencing can be performed on the complete gene or on specific domains thereof, such as those known or suspected to carry deleterious mutations or other alterations.

Amplification. Amplification is based on the formation of specific hybrids between complementary nucleic acid sequences that serve to initiate nucleic acid reproduction. Amplification can be performed according to various techniques known in the art, such as by polymerase chain reaction (PCR), ligase chain reaction (LCR), strand displacement amplification (SDA) and nucleic acid sequence based amplification (NASBA). These techniques can be performed using commercially available reagents and protocols. Useful techniques in the art encompass real-time PCR, allele-specific PCR, or PCR-SSCP. Amplification usually requires the use of specific nucleic acid primers, to initiate the reaction. For example, nucleic acid primers useful for amplifying sequences from the gene or locus of Nkx3.1 are able to specifically hybridize with a portion of the gene locus that flanks a target region of the locus, wherein the target region is present in subjects having or are at risk of developing prostate cancer.

The invention provides for a nucleic acid primer, wherein the primer can be complementary to and hybridize specifically to a portion of a coding sequence (e.g., gene or RNA) of Nkx3.1 that is present in subjects having or at risk of developing prostate cancer. Primers of the invention are specific for sequences in a gene or RNA of Nkx3.1. By using such primers, the detection of an amplification product indicates the presence of the Nkx3.1gene or the absence of such. Examples of primers of this invention can be single-stranded nucleic acid molecules of about 5 to 60 nucleotides in length, or about 8 to about 25 nucleotides in length. The sequence can be derived directly from the sequence of Nkx3.1, for example SEQ ID NO: 20. Perfect complementarity is useful, to ensure high specificity. However, certain mismatch can be tolerated. For example, a nucleic acid primer or a pair of nucleic acid primers as described herein can be used in a method for detecting the presence of or a predisposition to prostate cancer in a subject.

Amplification methods include, e.g., polymerase chain reaction, PCR (PCR PROTOCOLS, A GUIDE TO METHODS AND APPLICATIONS, ed. Innis, Academic Press, N.Y., 1990 and PCR STRATEGIES, 1995, ed. Innis, Academic Press, Inc., N.Y., ligase chain reaction (LCR) (see, e.g., Wu, Genomics 4:560, 1989; Landegren, Science 241:1077, 1988; Barringer, Gene 89:117, 1990); transcription amplification (see, e.g., Kwoh, Proc. Natl. Acad. Sci. USA 86:1173, 1989); and, self-sustained sequence replication (see, e.g., Guatelli, Proc. Natl. Acad. Sci. USA 87:1874, 1990); Q Beta replicase amplification (see, e.g., Smith, J. Clin. Microbiol. 35:1477-1491, 1997), automated Q-beta replicase amplification assay (see, e.g., Burg, Mol. Cell. Probes 10:257-271, 1996) and other RNA polymerase mediated techniques (e.g., NASBA, Cangene, Mississauga, Ontario); see also Berger, Methods Enzymol. 152:307-316, 1987; Sambrook; Ausubel; U.S. Pat. Nos. 4,683,195 and 4,683,202; Sooknanan, Biotechnology 13:563-564, 1995. All the references stated above are incorporated by reference in their entireties.

Hybridization. Hybridization detection methods are based on the formation of specific hybrids between complementary nucleic acid sequences that serve to detect nucleic acid sequences. A detection technique involves the use of a nucleic acid probe specific for wild type gene or RNA. The probe can be in suspension or immobilized on a substrate or support (for example, as in nucleic acid array or chips technologies). For example, a sample from the subject can be contacted with a nucleic acid probe specific for wild type Nkx3.1. According to the invention, a probe can be a polynucleotide sequence which is complementary to and specifically hybridizes with a, or a target portion of a, Nkx3.1 gene or RNA. Useful probes are those that are complementary to the Nkx3.1 gene, RNA, or target portion thereof. Probes can comprise single-stranded nucleic acids of between 8 to 1000 nucleotides in length, for instance between 10 and 800, between 15 and 700, or between 20 and 500. Longer probes can be used as well. A useful probe of the invention is a single stranded nucleic acid molecule of between 8 to 500 nucleotides in length, which can specifically hybridize to a region of a gene or RNA.

The sequence of the probes can be derived from the sequences of Nkx3.1 genes. Nucleotide substitutions can be performed, as well as chemical modifications of the probe. Such chemical modifications can be accomplished to increase the stability of hybrids (e.g., intercalating groups) or to label the probe. Some examples of labels include, without limitation, radioactivity, fluorescence, luminescence, and enzymatic labeling.

A guide to nucleic acid hybridization is found in e.g., Sambrook, ed., Molecular Cloning: A Laboratory Manual (3^{rd} Ed.), Vols. 1-3, Cold Spring Harbor Laboratory, 2001; CURRENT PROTOCOLS IN MOLECULAR BIOLOGY, Ausubel, ed. John Wiley & Sons, Inc., New York, 1997; LABORATORY TECHNIQUES IN BIOCHEMISTRY AND MOLECULAR BIOLOGY: HYBRIDIZATION WITH NUCLEIC ACID PROBES, PART I. Theory and Nucleic Acid Preparation, Tijssen, ed. Elsevier, N.Y., 1993.

Specific Ligand Binding. As indicated herein, the presence of a Nkx3.1 gene locus or Nkx3.1 expression can also be detected. Different types of ligands can be used, such as specific antibodies. In one embodiment, the sample is contacted with an antibody specific for a Nkx3.1 and the formation of an immune complex is subsequently determined. Various methods for detecting an immune complex can be used, such as ELISA, radioimmunoassays (RIA) and immuno-enzymatic assays (IEMA).

For example, an antibody can be a polyclonal antibody, a monoclonal antibody, as well as fragments or derivatives thereof having substantially the same antigen specificity. Fragments include Fab, Fab′2, or CDR regions. Derivatives include single-chain antibodies, humanized antibodies, or poly-functional antibodies. An antibody specific for a Nkx3.1 polypeptide can be an antibody that selectively binds Nkx3.1, namely, an antibody raised against a Nkx3.1 polypeptide or an epitope-containing fragment thereof. Although non-specific binding towards other antigens can occur, binding to the target polypeptide occurs with a higher affinity and can be reliably discriminated from non-specific binding. In one embodiment, the method comprises contacting a sample from the subject with an antibody specific for a wild type Nkx3.1 polypeptide, and determining the presence of an immune complex. Optionally, the sample can be contacted to a support coated with antibody specific for the wild type Nkx3.1 polypeptide.

The invention also provides for a diagnostic kit comprising products and reagents for detecting in a sample from a subject the presence of a Nkx3.1 gene, or a Nkx3.1 polypeptide, and/or the presence of homeobox 1 transcription factor activity. The kit can be useful for determining whether a sample from a subject exhibits Nkx3.1 expression, for example lumincal prostate stem cells, such as CARNs. For example, the diagnostic kit according to the present invention comprises any primer, any pair of primers, any nucleic acid probe and/or any ligand, (for example, an antibody directed to Nkx3.1). The diagnostic kit according to the present invention can further comprise reagents and/or protocols for performing a hybridization, amplification or antigen-antibody immune reaction. In one embodiment, the kit can comprise nucleic acid primers that specifically hybridize to and can prime a polymerase reaction from Nkx3.1. In another embodiment, the primer can comprise a nucleotide sequence of SEQ ID NOS: 9 or 11. In one embodiment, the presence of Nkx3.1 can be detected in a population of prostate stem cells, such as CARNs.

The diagnosis methods can be performed in vitro, ex vivo, or in vivo. These methods utilize a sample from a subject in order to assess the status of the Nkx3.1 gene locus. The sample can be any biological sample derived from a subject, which contains nucleic acids or polypeptides. Examples of such samples include, but are not limited to, fluids, tissues, cell samples, organs, or tissue biopsies. In one embodiment, the sample comprises prostate tissue. In another embodiment, the sample is an isolated population of prostate stem cells. The sample can be collected according to conventional techniques and used directly for diagnosis or stored. The sample can be treated prior to performing the method, in order to render or improve availability of nucleic acids or polypeptides for testing. Treatments include, for instance, lysis (e.g., mechanical, physical, or chemical), centrifugation. Also, the nucleic acids and/or polypeptides can be pre-purified or enriched by conventional techniques, and/or reduced in complexity. Nucleic acids and polypeptides can also be treated with enzymes or other chemical or physical treatments to produce fragments thereof. In one embodiment, the sample is contacted with reagents, such as probes, primers, or ligands, in order to assess the presence of Nkx3.1, for example, in a population of luminal prostate stem cells, such as CARNs. Contacting can be performed in any suitable device, such as a plate, tube, well, or glass. In specific embodiments, the contacting is performed on a substrate coated with the reagent, such as a nucleic acid array or a specific ligand array. The substrate can be a solid or semi-solid substrate such as any support comprising glass, plastic, nylon, paper, metal, or polymers. The substrate can be of various forms and sizes, such as a slide, a membrane, a bead, a column, or a gel. The contacting can be made under any condition suitable for a complex to be formed between the reagent and the nucleic acids or polypeptides of the sample.

Identifying a polypeptide, RNA or DNA of Nkx3.1 in the sample can be correlated to the presence, predisposition or stage of progression of prostate cancer. For example, an individual expressing Nkx3.1 has an increased risk of developing prostate cancer. The determination of the presence of Nkx3.1 in a subject also allows the design of appropriate therapeutic intervention, which is more effective and customized. Also, this determination at the pre-symptomatic level allows a preventive regimen to be applied.

Methods of Drug Targeting

The present invention provides methods for targeting a therapeutic agent to a stem cell (e.g., a CARN) in a subject by conjugating a therapeutic agent to an agent that binds to Nkx3.1 and administering the conjugated agent to the subject. These methods can be used to target therapeutic agents, such as drugs, to Nkx3.1-positive cells (e.g., CARNs or prostate cancer cells, or luminal prostate stem cells). For example, therapeutic agents that may be targeted to Nkx3.1-positive cells include, but are not limited to, cytotoxic drugs, other toxins and radionuclides. Conjugates can be useful where Nkx3.1-positive cells are Nkx3.1-positive cancer cells, CARNs, or other Nkx3.1-positive cells that are over-proliferative. In some embodiments, the therapeutic agents are conjugated to an antibody that binds to Nkx3.1. For example, the antibody can be a monoclonal antibody directed to Nkx3.1 (such as a humanized monoclonal antibody), or a polyclonal antibody directed to Nkx3.1. Methods of conjugating therapeutic agents to antibodies are known in the art, and any such method can be used.

The invention provides for methods used to identify compounds that inhibit prostate cancer. For example, the stem cell would be maintained in a de-differentiated state. In one embodiment, the method comprises contacting a population of luminal, prostate epithelium stem cells with a test compound under culture conditions which would cause differentiation of the stem cells into prostate cancer cells. The method can further comprise determining whether the differentiation of prostate cancer cells is inhibited in the presence of the test compound as compared to differentiation of the stem cells in the absence of the test compound. Test compounds that modulate the function of Nkx3.1 can be useful. In one embodiment, the present invention is directed to agents that modulate the function of Nkx3.1 and to methods of identifying such compounds. These test compounds can be useful as anti-tumor drugs, or as agents for maintaining stem cells in culture, or as agents for facilitating differentiation of stem cells into differentiated cells types.

Test compounds can be screened from large libraries of synthetic or natural compounds (see Wang et al., (2007) Curr Med Chem, 14(2):133-55; Mannhold (2006) Curr Top Med Chem, 6 (10):1031-47; and Hensen (2006) Curr Med Chem 13(4):361-76). Numerous means are currently used for random and directed synthesis of saccharide, peptide, and nucleic acid based compounds. Synthetic compound libraries are commercially available from Maybridge Chemical Co. (Trevillet, Cornwall, UK), Comgenex (Princeton, N.J.), Brandon Associates (Merrimack, N.H.), and Microsource (New Milford, Conn.). A rare chemical library is available from Aldrich (Milwaukee, Wis.). Alternatively, libraries of natural compounds in the form of bacterial, fungal, plant and animal extracts are available from e.g. Pan Laboratories (Bothell, Wash.) or MycoSearch (N.C.), or are readily producible. Additionally, natural and synthetically produced libraries and compounds are readily modified through conventional chemical, physical, and biochemical means (Blondelle et al., (1996) Tib Tech 14:60).

Methods for preparing libraries of molecules are well known in the art and many libraries are commercially available. Libraries of interest in the invention include peptide libraries, randomized oligonucleotide libraries, synthetic organic combinatorial libraries, and the like. Degenerate peptide libraries can be readily prepared in solution, in immobilized form as bacterial flagella peptide display libraries or as phage display libraries. Peptide ligands can be selected from combinatorial libraries of peptides containing at least one amino acid. Libraries can be synthesized of peptoids and non-peptide synthetic moieties. Such libraries can further be synthesized which contain non-peptide synthetic moieties, which are less subject to enzymatic degradation compared to their naturally-occurring counterparts. Libraries are also meant to include for example but are not limited to peptide-on-plasmid libraries, polysome libraries, aptamer libraries, synthetic peptide libraries, synthetic small molecule libraries, neurotransmitter libraries, and chemical libraries. The libraries can also comprise cyclic carbon or heterocyclic structure and/or aromatic or polyaromatic structures substituted with one or more of the functional groups.

Small molecule combinatorial libraries can also be generated and screened. A combinatorial library of small organic compounds is a collection of closely related analogs that differ from each other in one or more points of diversity and are synthesized by organic techniques using multi-step processes. Combinatorial libraries include a vast number of small organic compounds. One type of combinatorial library is prepared by means of parallel synthesis methods to produce a compound array. A compound array can be a collection of compounds identifiable by their spatial addresses in Cartesian coordinates and arranged such that each compound has a common molecular core and one or more variable structural diversity elements. The compounds in such a compound array are produced in parallel in separate reaction vessels, with each compound identified and tracked by its spatial address. Examples of parallel synthesis mixtures and parallel synthesis methods are provided in U.S. Ser. No. 08/177,497, filed Jan. 5, 1994 and its corresponding PCT published patent application WO95/18972, published Jul. 13, 1995 and U.S. Pat. No. 5,712,171 granted Jan. 27, 1998 and its corresponding PCT published patent application WO96/22529, which are hereby incorporated by reference.

Examples of chemically synthesized libraries are described in Fodor et al., (1991) Science 251:767-773; Houghten et al., (1991) Nature 354:84-86; Lam et al., (1991) Nature 354:82-84; Medynski, (1994) BioTechnology 12:709-710; Gallop et al., (1994) J. Medicinal Chemistry 37(9):1233-1251; Ohlmeyer et al., (1993) Proc. Natl. Acad. Sci. USA 90:10922-10926; Erb et al., (1994) Proc. Natl. Acad. Sci. USA 91:11422-11426; Houghten et al., (1992) Biotechniques 13:412; Jayawickreme et al., (1994) Proc. Natl. Acad. Sci. USA 91:1614-1618; Salmon et al., (1993) Proc. Natl. Acad. Sci. USA 90:11708-11712; PCT Publication No. WO 93/20242, dated Oct. 14, 1993; and Brenner et al., (1992) Proc. Natl. Acad. Sci. USA 89:5381-5383.

Screening the libraries can be accomplished by any variety of commonly known methods. See, for example, the following references, which disclose screening of peptide libraries: Parmley and Smith, (1989) Adv. Exp. Med. Biol. 251:215-218; Scott and Smith, (1990) Science 249:386-390; Fowlkes et al., (1992) BioTechniques 13:422-427; Oldenburg et al., (1992) Proc. Natl. Acad. Sci. USA 89:5393-5397; Yu et al., (1994) Cell 76:933-945; Staudt et al., (1988) Science 241:577-580; Bock et al., (1992) Nature 355:564-566; Tuerk et al., (1992) Proc. Natl. Acad. Sci. USA 89:6988-6992; Ellington et al., (1992) Nature 355:850-852; U.S. Pat. Nos. 5,096,815; 5,223,409; and 5,198,346, all to Ladner et al.; Rebar et al., (1993) Science 263:671-673; and PCT Pub. WO 94/18318.

Cancer Stem Cells

The present invention provides methods involving prostate cancer cells. These methods are based on the discovery that Nkx3.1 is a marker of cancer stem cells, such as those giving rise to prostate cancer. For example, CARNs, a population of luminal prostate stem cells, expresses Nkx3.1, are cells of origin for prostate cancer. In one embodiment, the present invention provides a method of detecting a cancer stem cell comprising contacting a tissue, tissue sample or cell population with an agent that binds to Nkx3.1 and determining whether the agent has bound to the tissue, tissue sample, or cell population. For example, binding of the agent indicates the presence of a cancer stem cell and absence of binding indicates an absence of cancer stem cells. In another embodiment, the invention is directed to methods for detecting a tumor comprising. For example, the method can comprise contacting a tissue, tissue sample, or cell population with an agent that binds to Nkx3.1 and further determining whether the agent has bound to the tissue, tissue sample, or cell population. The binding of the agent indicates the presence of tumor cells and an absence of such agent binding indicates an absence of tumor cells.

The present invention is also directed to methods for determining whether a subject is likely to develop cancer, by determining whether a tissue, tissue sample, or cell population from the subject contains one or more Nkx3.1-positive cancer stem cells or tumor cells. The presence of such cells may provide an early prognostic marker, and thus be useful for detecting tumors, or subjects likely to develop tumors, at an early stage, allowing appropriate preventative or therapeutic regimens to be initiated early.

The drug targeting methods described herein, are useful to use with Nkx3.1-positive cancer cells. Such methods can be used to target chemotherapeutic drugs, radionuclide drugs, or other toxic agents to Nkx3.1-positive cancer stem cells, thereby killing the Nkx3.1-positive cancer stem cells but not the surrounding non-cancerous tissue.

Unless otherwise defined, all technical and scientific terms used herein have the same meaning as commonly understood by one of ordinary skill in the art to which this invention belongs. Exemplary methods and materials are described below, although methods and materials similar or equivalent to those described herein can also be used in the practice or testing of the present invention.

All publications and other references mentioned herein are incorporated by reference in their entirety, as if each individual publication or reference were specifically and individually indicated to be incorporated by reference. Publications and references cited herein are not admitted to be prior art.

### Examples

Examples are provided below to facilitate a more complete understanding of the invention. The following examples illustrate the exemplary modes of making and practicing the invention. However, the scope of the invention is not limited to specific embodiments disclosed in these Examples, which are for purposes of illustration only, since alternative methods can be utilized to obtain similar results.

### Example 1

### A Luminal Epithelial Stem Cell that is a Cell of Origin for Prostate Cancer

In epithelial tissues, the lineage relationship between normal progenitor cells and cell type(s) of origin for cancer has been poorly understood. Here we show that a known regulator of prostate epithelial differentiation, the homeobox gene Nkx3.1, marks a stem cell population that functions during prostate regeneration. Genetic lineage-marking demonstrates that rare luminal cells which express Nkx3.1 in the absence of testicular androgens (castration-resistant Nkx3.1-expressing cells, CARNs) are bipotential and can self-renew in vivo, while single-cell transplantation assays show that CARNs can reconstitute prostate ducts in renal grafts. Functional assays of Nkx3.1 mutant mice in serial prostate regeneration assays suggest that Nkx3.1 is required for stem cell maintenance. Finally, targeted deletion of the Pten tumor suppressor gene in CARNs results in rapid formation of carcinoma following androgen-mediated regeneration. These observations indicate that CARNs represent a luminal stem cell population that is an efficient target for oncogenic transformation in prostate cancer.

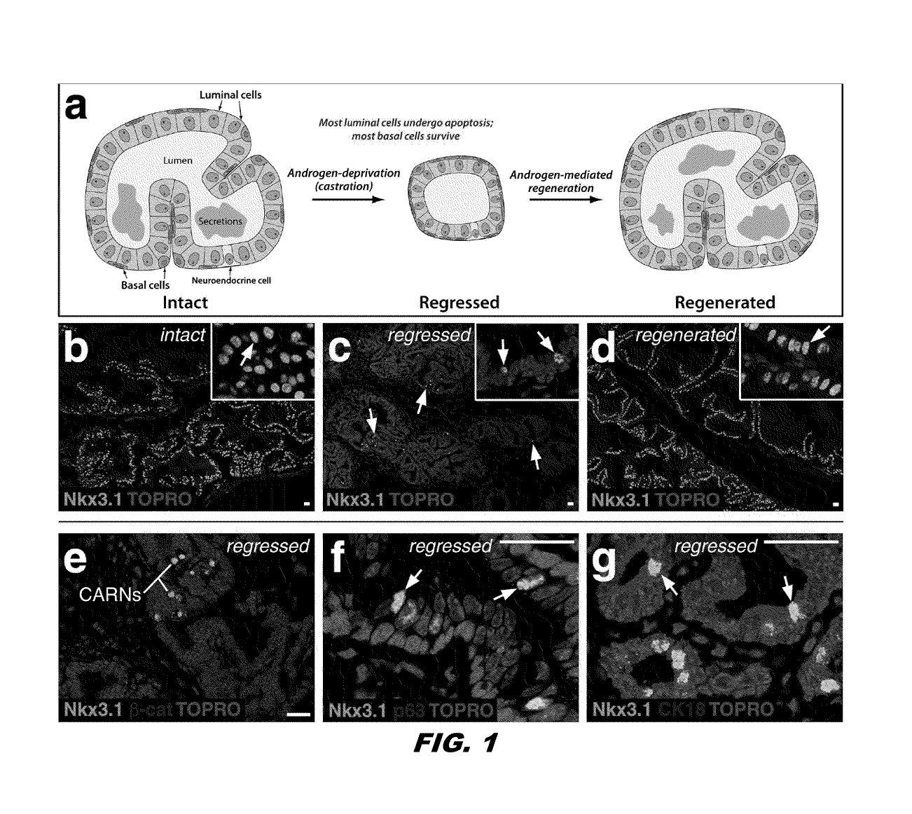

The prostate represents an excellent system for studying the function and molecular regulation of adult epithelial stem cells in the context of both tissue regeneration and cancer. The prostate epithelium is comprised of three differentiated cell types: luminal secretory cells, basal cells, and neuroendocrine cells (FIG. 1A)¹. Androgen-deprivation leads to rapid apoptosis of approximately 90% of luminal cells and a small percentage of basal cells, although a stable cell number is maintained in the regressed state^{2,3}. Following re-administration of androgens, the prostate epithelium regenerates over approximately two weeks²⁻⁴, and is capable of more than 15 rounds of serial regression/regeneration^{5,6}, implying that the prostate epithelium contains a longterm population of castration-resistant stem cells.

Substantial evidence supports the existence of a basal stem cell population in the prostate⁷, consistent with analyses of progenitor cells in other epithelial tissues⁸. In particular, subpopulations of basal cells isolated using cell-surface markers display bipotentiality and self-renewal in explant culture and tissue grafts⁹⁻¹³. Furthermore, single Lin⁺Sca-1⁺CD133⁺CD44⁺CD117⁺ cells, which are predominantly basal in the mouse and exclusively basal in the human, can reconstitute prostatic ducts in renal grafts¹⁴. However, explants from p63 null mice can form prostate tissue and undergo multiple rounds of serial regression/regeneration in the absence of basal cells¹⁵, suggesting the existence of a distinct luminal stem cell population. To date, however, luminal stem cells have not been identified in the prostate or other stratified epithelial tissues.

Although basal stem/progenitor cells have been proposed to represent a cell type of origin^{7,16,17}, human prostate cancer has a strikingly luminal phenotype. Notably, the absence of basal cells is a diagnostic feature for prostate adenocarcinoma^{18,19}, suggesting either that prostate cancer arises from a luminal cell, or that oncogenic transformation of a basal progenitor results in rapid differentiation of luminal progeny. Here we show that expression of the Nkx3.1 homeobox gene in the androgen-deprived prostate epithelium marks a rare luminal cell population that displays stem/progenitor properties during prostate regeneration. Our findings also indicate the relevance of this luminal stem cell population as a cell type of origin for prostate cancer.

Detection of CARNs in the Prostate

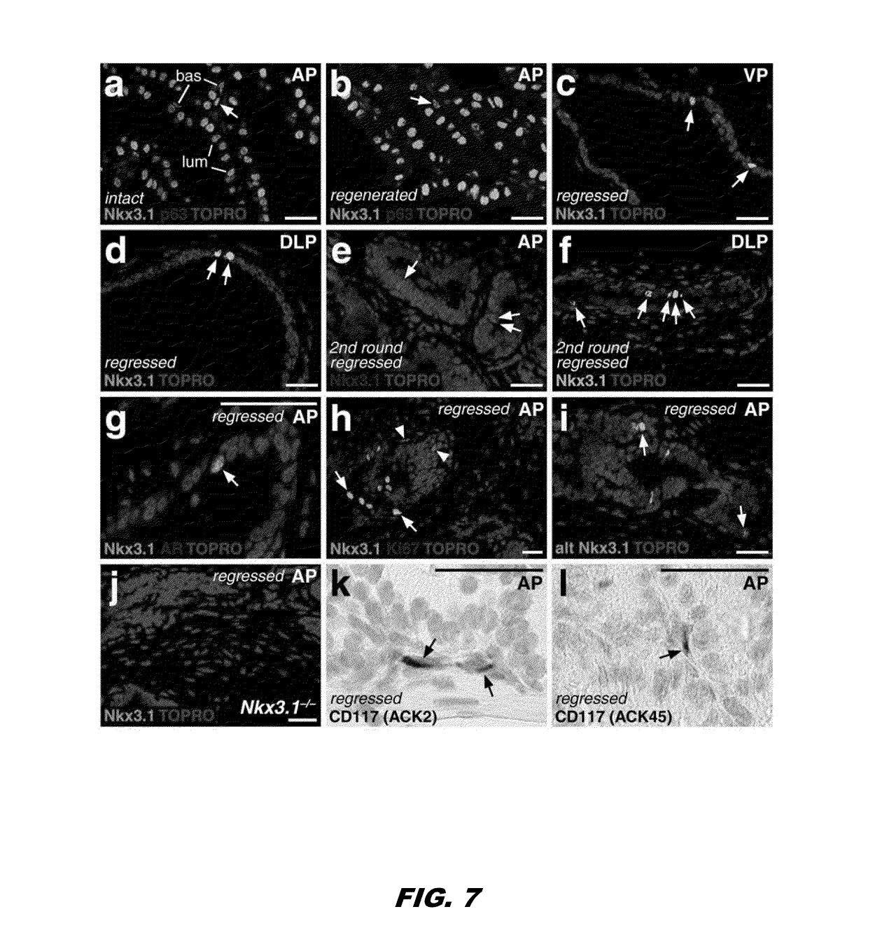

The Nkx3.1 homeobox gene regulates prostate epithelial differentiation, and is frequently inactivated at early stages of prostate tumorigenesis²⁰. Notably, Nkx3.1 homozygous mutant mice develop prostatic intraepithelial neoplasia (PIN), a precursor of prostate cancer, by one year of age²¹⁻²³. In the intact adult mouse prostate, all luminal cells express Nkx3.1, while 9.5% of p63⁺ basal cells (n=4291) also express Nkx3.1 (FIG. 1B; FIG. 7A)²⁴. Previous studies have shown that Nkx3.1 expression in prostate epithelial cells is reduced or abolished in the absence of androgens in vivo, and is consequently androgen-dependent^{25,26}. Thus, Nkx3.1 expression is rapidly lost following castration, while Nkx3.1 expression is quickly restored after androgen re-administration to induce prostate regeneration (FIG. 1C, 1D; FIG. 7B).

However, Nkx3.1 expression is not completely absent in the regressed prostate, but is instead retained in a rare population of epithelial cells (FIG. 1C, 1E). These CAstration-Resistant Nkx3.1-expressing cells (CARNs) comprise 0.7% of total epithelial cells (n=38,329) in the anterior prostate of androgen-deprived males, or approximately 460 CARNs per mouse (Table 1). In addition, CARNs are frequently clustered (FIG. 1E), and can be detected in the ventral and dorsal prostate, as well as after a second round of regression (FIG. 7C-7F).

TABLE 1
Summary of properties of CARNs.
1. CARNs are a rare cell population

| | # Epithelial cells | # Mice | # CARNs (Nkx3.1⁺) |
|---|---|---|---|
| Regressed prostate | 38,329 | 4 | 266 (0.7%) |

2. CARNs are luminal cells

| | | | | |
|---|---|---|---|---|
| Marker | Cell type | # CARNs examined | # Mice | # CARNs expressing |

a. Luminal phenotype of CARNs

| | | | | |
|---|---|---|---|---|
| p63 | basal | 379 | 9 | 0 (0%) |
| Cytokeratin 18 | luminal | 837 | 4 | 828 (99%) |
| Synaptophysin | neuroendocrine | 610 | 5 | 0 (0%) |

b. Luminal phenotype of lineage-marked CARNs

| | | | | |
|---|---|---|---|---|
| p63 | basal | 98 | 3 | 0 (0%) |
| Cytokeratin 14 | basal | 131 | 4 | 0 (0%) |
| Cytokeratin 5 | basal | 93 | 4 | 2 (2%) |
| Cytokeratin 18 | luminal | 123 | 4 | 123 (100%) |

3. CARNs are bipotential

| | # YFP⁺ cells | | # Basal YFP⁺ cells (basal |
|---|---|---|---|
| | (lineage-marked progeny) | # Mice | lineage-marked progeny) |
| Progeny of lineage- | 559 | 4 | 17 (3.0%) |
| marked CARNs in | | | |
| regenerated prostate | | | |

4. CARNs can self-renew

| | # Nkx3.1⁺YFP⁺ cells | | # Nkx3.1⁺YFP⁺BrdU⁺ cells |
|---|---|---|---|
| | (lineage-marked CARNs) | # Mice | (self-renewing CARNs) |
| Self-renewing lineage- | 68 | 4 | 16 (24%) |
| marked CARNs in | | | |
| regressed prostate | | | |

5. CARNs can reconstitute prostate ducts after single-cell transplantation

| Grafted cells | Grafts performed | Grafts recovered |
|---|---|---|
| Single YFP-positive | 43 | 16 (37%) |
| (lineage-marked CARN) | | (p < 0.001) |
| Single YFP-negative | 31 | 1 (3%) |

Importantly, all CARNs in the regressed prostate are strictly luminal, since they never express the basal cell marker p63 (n=0/379) or the neuroendocrine marker synaptophysin (n=0/610) (FIG. 1F; Table 1). Instead, CARNs express the luminal markers cytokeratin 18 (CK18) (n=828/837) and androgen receptor (AR) (n=46/46) and are growth-quiescent, as they do not co-express Ki67 (n=0/151) (FIG. 1G; FIG. 7G, 7H). The CARNs population is non-overlapping with the Lin⁻Sca-1⁺CD133⁺CD44⁻CD117⁺ stem/progenitor population¹⁴, since CD117 (c-kit) positive cells in regressed prostate are never luminal (n=0/79) (FIG. 7K, 7L). Furthermore, since the CARNs population is strictly luminal, it is also distinct from other previously described prostate stem cell populations that are exclusively basal^{9,10,13}.

Bipotentiality and Self-Renewal

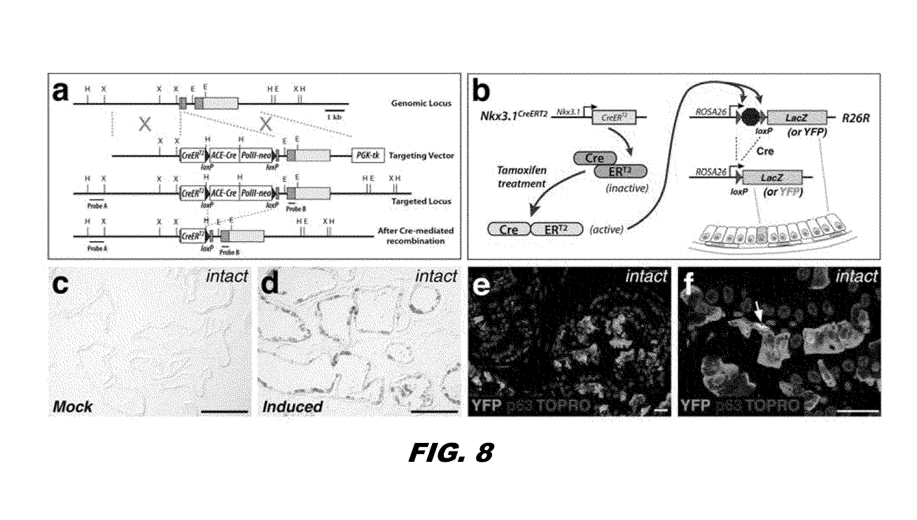

To investigate whether the CARNs population might correspond to prostate epithelial progenitors, we performed in vivo lineage-marking using a knock-in allele that places a tamoxifen-inducible Cre recombinase^{27,28} under the transcriptional control of the Nkx3.1 promoter (FIG. 8A). We assessed the specificity of the Nkx3.1^{CreERT2} allele in control crosses with the R26R-YFP Cre-reporter²⁹ and the R26R-lacZ alleles³⁰, and found that Cre-mediated recombination following tamoxifen administration faithfully recapitulates the endogenous pattern of Nkx3.1 expression in the intact prostate (FIG. 8B-8F).

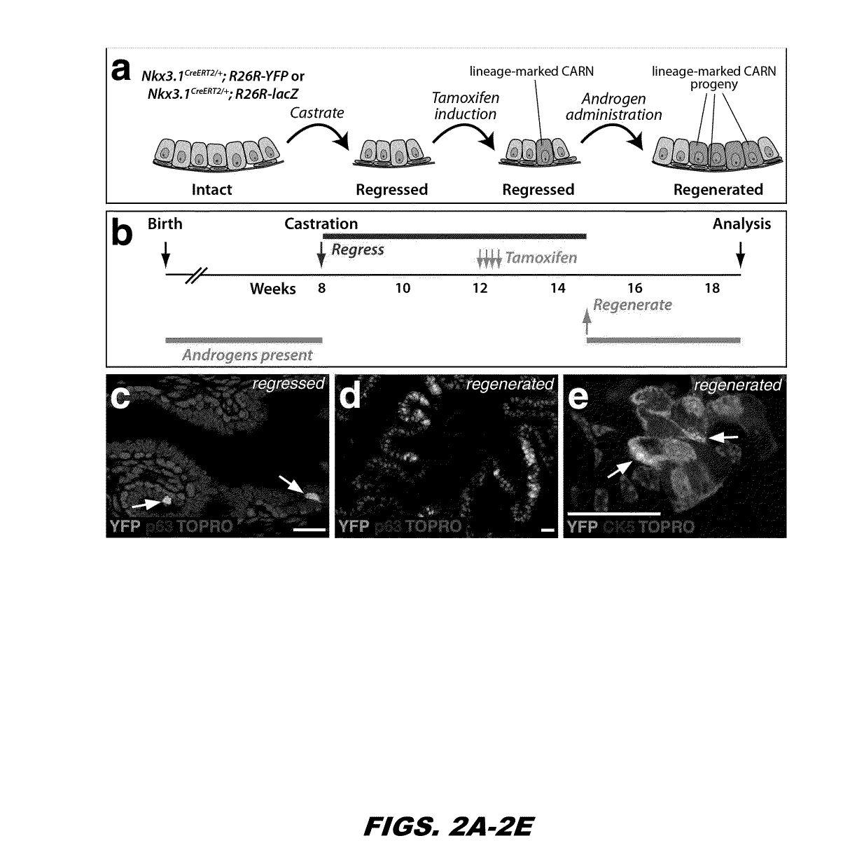

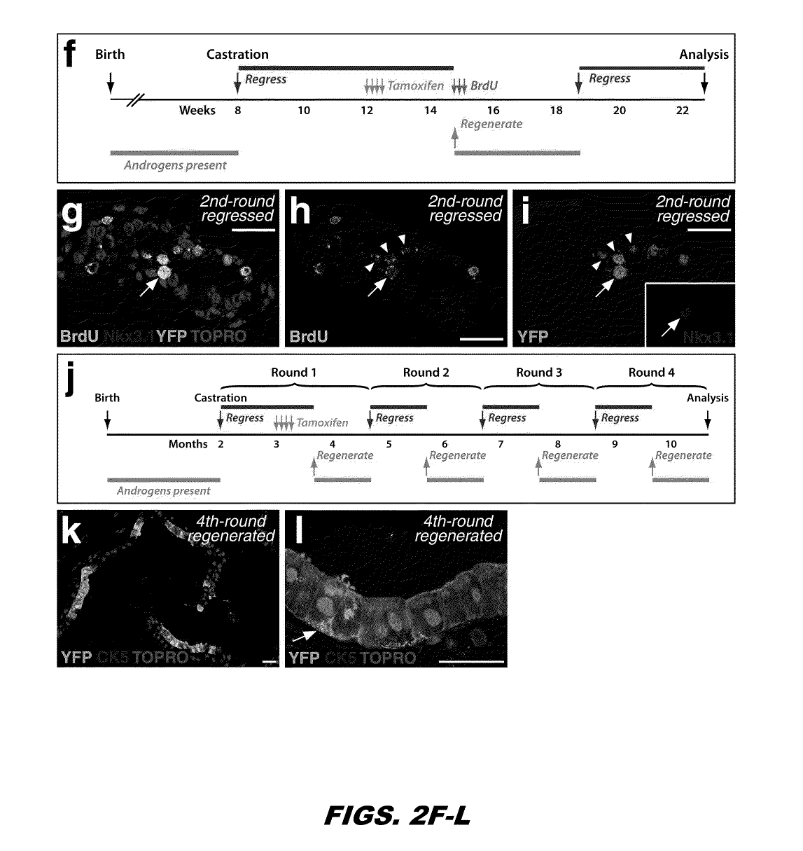

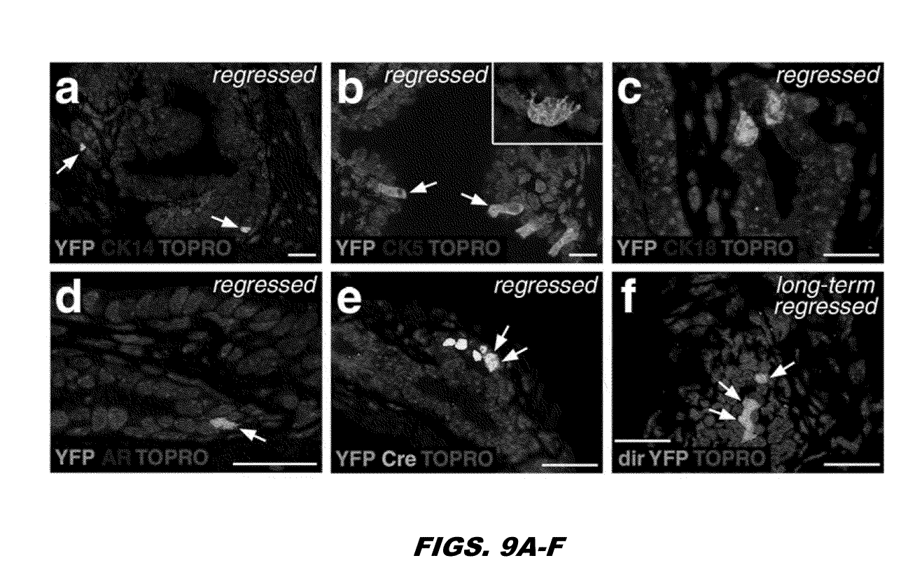

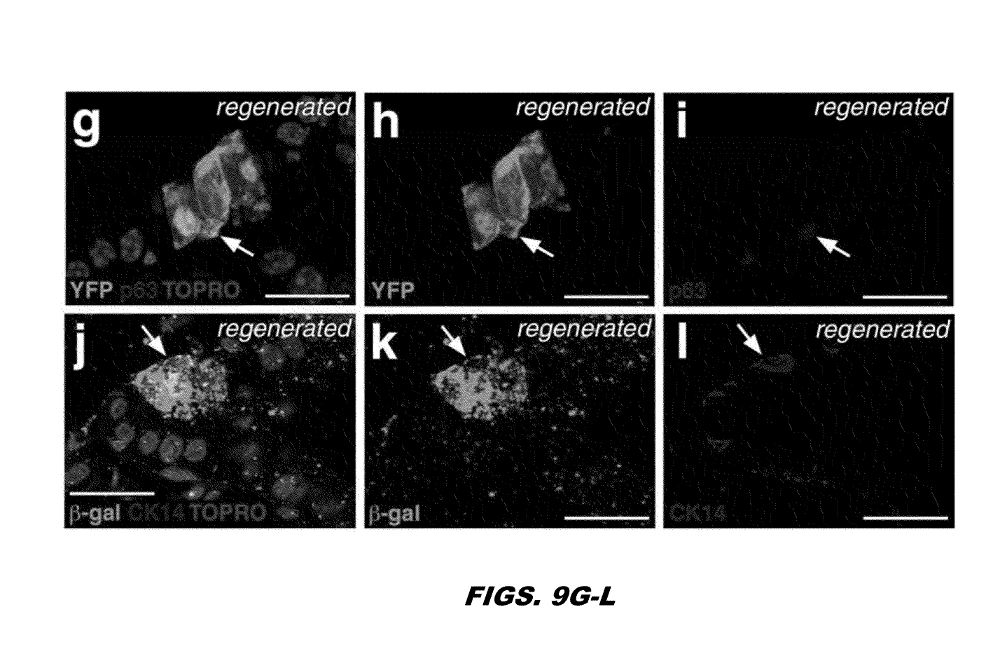

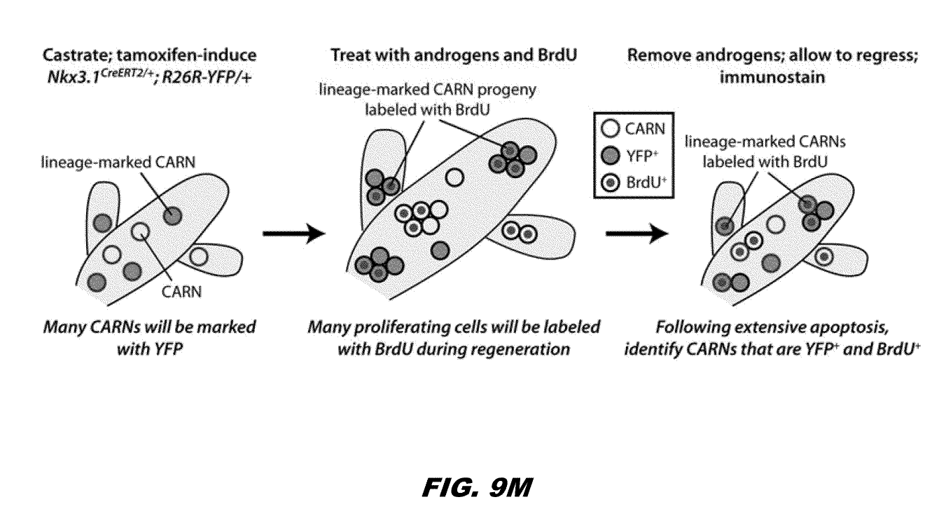

We performed lineage-marking of CARNs by tamoxifen treatment of castrated Nkx3.1^{CreERT2/+}; R26R-YFP/+ or Nkx3.1^{CreERT2/+}; R26R-lacZ/+ adult males (FIG. 2A, 2B). For genetic marking of CARNs in regressed prostate, we observed YFP fluorescence/β-galactosidase expression in rare epithelial cells that were strictly luminal (Table 1). These lineage-marked cells were never positive for the basal markers p63 (n=0/98) or CK14 (n=0/131), and almost never positive for CK5 (n=2/93), but always expressed the luminal markers CK18 (n=123/123) and AR (n=94/94) (FIG. 2C; FIG. 9A-D). Following regeneration, the percentage of lineage-marked cells increased 9-fold (from 0.37% (n=19,825) to 3.3% (n=95,017), p<0.0001) (FIG. 2D), indicating the proliferative potential of CARNs. Although most of the lineage-marked cells in regenerated prostates were luminal, we observed occasional YFP⁺CK5⁺, YFP⁺p63⁺, or β-gal⁺CK14⁻ basal cells, corresponding to 3.0% of lineage-marked cells (n=559) (FIG. 2E; FIG. 9G-L); this percentage of regenerated basal cells is consistent with the low percentage of basal cells lost during regression². Since all of the lineage-marked cells were luminal in the regressed prostate, but could give rise to both basal and luminal cells during regeneration, we conclude that the initial CARNs population contains bipotential progenitors.

To investigate the self-renewal of CARNs, we examined whether they could undergo at least one cell division during prostate regeneration to generate a daughter cell that is also a CARN. We determined whether lineage-marked CARNs in castrated Nkx3.1^{CreERT2/+}; R26R-YFP/+ mice would incorporate BrdU during prostate regeneration, while retaining CARN identity (Nkx3.1 expression) after a subsequent prostate regression (FIG. 2F; FIG. 9M). Such triple-positive Nkx3.1⁺YFP⁺BrdU⁺ cells were observed (FIG. 2G-I), providing evidence for CARN self-renewal. In particular, the percentage of BrdU⁺ cells among Nkx3.1⁺YFP⁺ cells, corresponding to CARNs in both the first and second regression, represents the percentage of CARNs undergoing a self-renewal division (24%, n=68; Table 2).

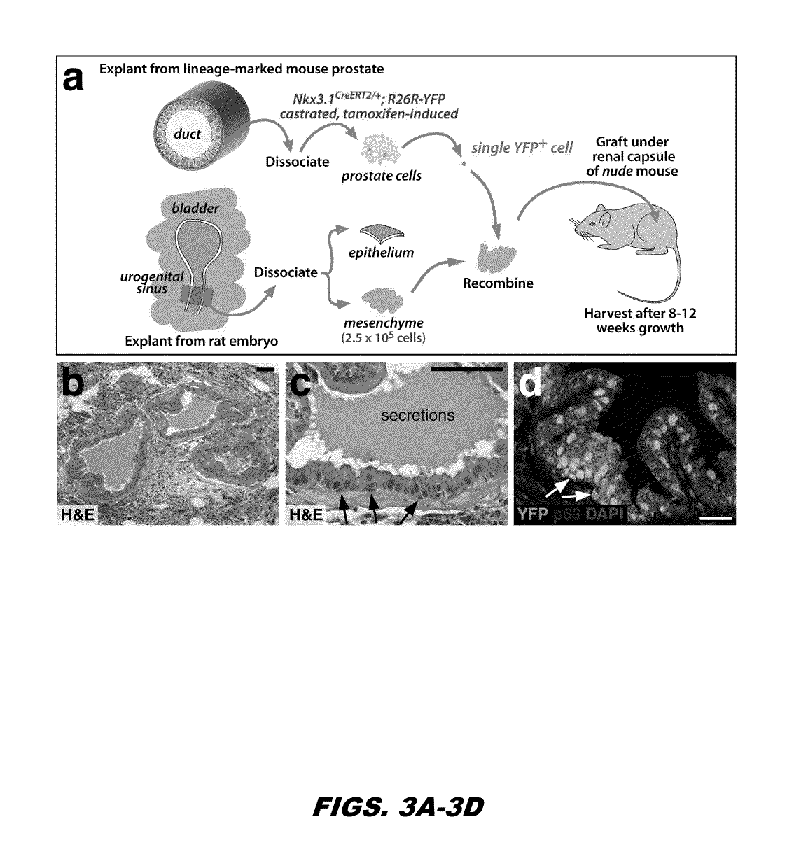

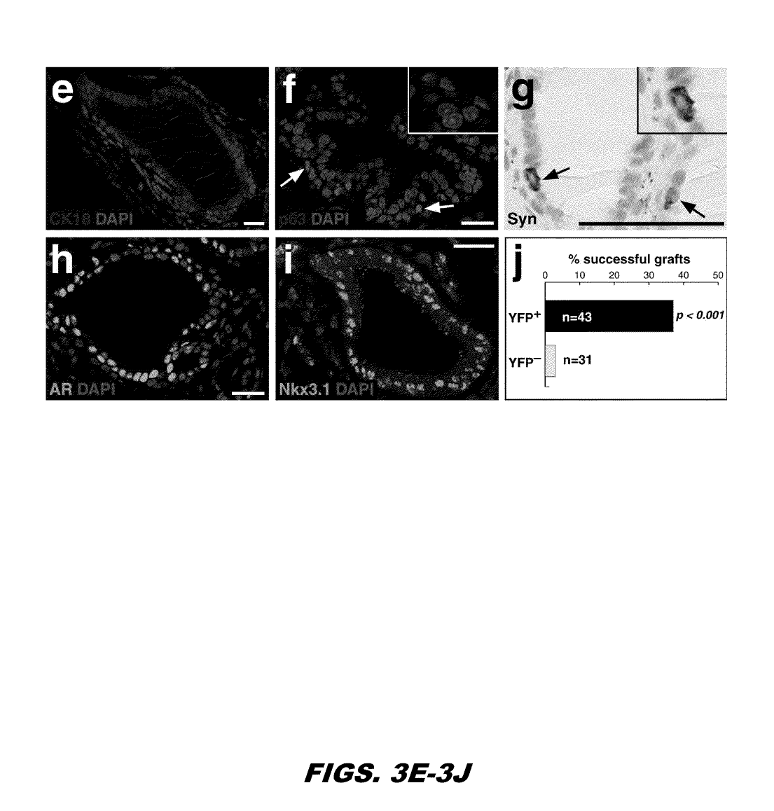

TABLE 2
Analysis of self-renewal in CARNs.

| Marker phenotype of cells in second | | Number of cells in category | | | | |
|---|---|---|---|---|---|---|
| round regressed state (FIG. 3a, b) | | Animal analyzed | | | | Total |
| Category of cells | Interpretation | #1 | #2 | #3 | #4 | (percent) |
| Nkx3.1⁺ | CARNs | 100 | 64 | 337 | 417 | 918 |
| | | | | | | (2.5%) |
| YFP⁺ | Lineage-marked cells | 169 | 108 | 330 | 378 | 985 |
| | | | | | | (2.6%) |
| BrdU⁺ | Proliferating cells | 318 | 196 | 816 | 920 | 2250 |
| | | | | | | (6.0%) |
| Nkx3.1⁺YFP⁺ | Lineage-marked | 13 | 12 | 19 | 24 | 68 |
| | CARNs (second | | | | | (0.18%) |
| | round) | | | | | |
| Nkx3.1⁺BrdU⁺ | CARNs that have | 14 | 24 | 58 | 70 | 166 |
| | proliferated | | | | | (0.44%) |
| YFP⁺BrdU⁺ | Lineage-marked cells | 27 | 20 | 35 | 45 | 127 |
| | that have proliferated | | | | | (0.34%) |
| Nkx3.1⁺YFP⁺BrdU⁺ | CARNs that have | 3 | 2 | 5 | 6 | 16 |
| | undergone a self- | | | | | (0.04%) |
| | renewal division | | | | | |
| Total epithelial cells | | 6259 | 3705 | 13484 | 14067 | 37515 |
| Ratios of cell categories | Interpretation | Percentages | | | | |
| Nkx3.1⁺BrdU⁺/Nkx3.1⁺ | Percentage of CARNs | 14% | 38% | 17% | 17% | 18% |
| | that have proliferated | | | | | |
| Nkx3.1⁺YFP⁺BrdU⁺/ | Percentage of lineage- | 23% | 17% | 26% | 25% | 24% |
| Nkx3.1⁺YFP⁺ | marked CARNs that | | | | | |
| | have undergone a self- | | | | | |
| | renewal division | | | | | |

Cells were counted in sections of anterior prostate from 4 different mice treated as shown in FIG. 2F. Regions enriched for CARNs (Nkx3.1⁺ cells) were analyzed; thus the percentage of CARNs (2.5%) is higher than that determined by counting sections through the entire prostate.

To assess long-term self-renewal, we examined the persistence of lineage-marked cells in Nkx3.1^{CreERT2/+}; R26R-YFP/+ mice after four rounds of regression/regeneration (FIG. 2J). In these mice, YFP⁺ cells represented 3.0% (n=21,559) of the prostate epithelium, similar to the percentage observed after one round (FIG. 2K, 2L). The persistence of YFP⁺ cells is consistent with the maintenance of a constant stem cell number during regeneration, as suggested by the ability of the epithelium to undergo apparently unlimited serial regeneration^{5,6}, and supports the long-term self-renewal of lineage-marked CARNs.

Single-Cell Transplantation of CARNs

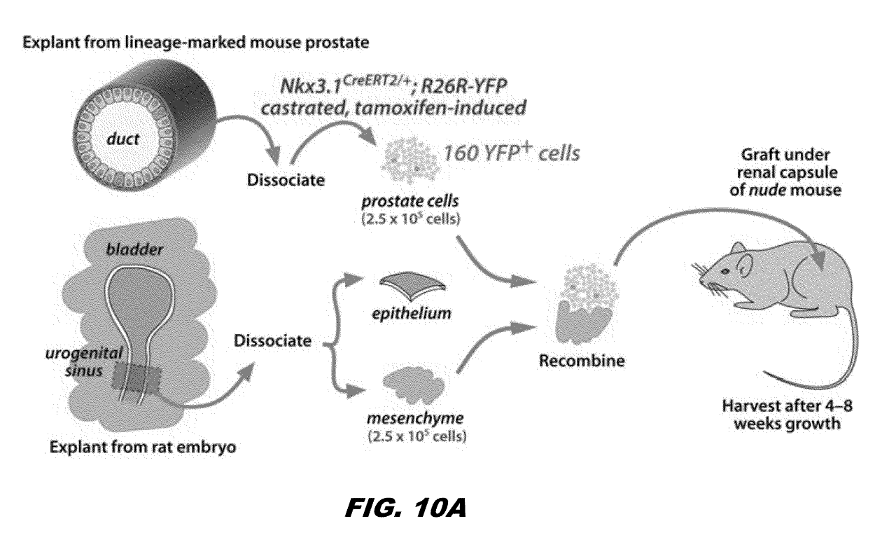

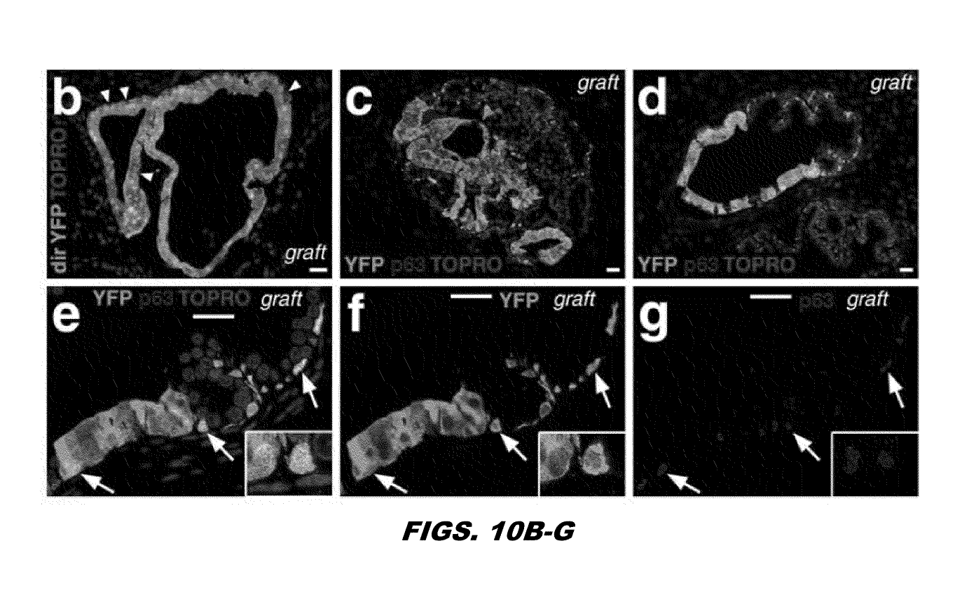

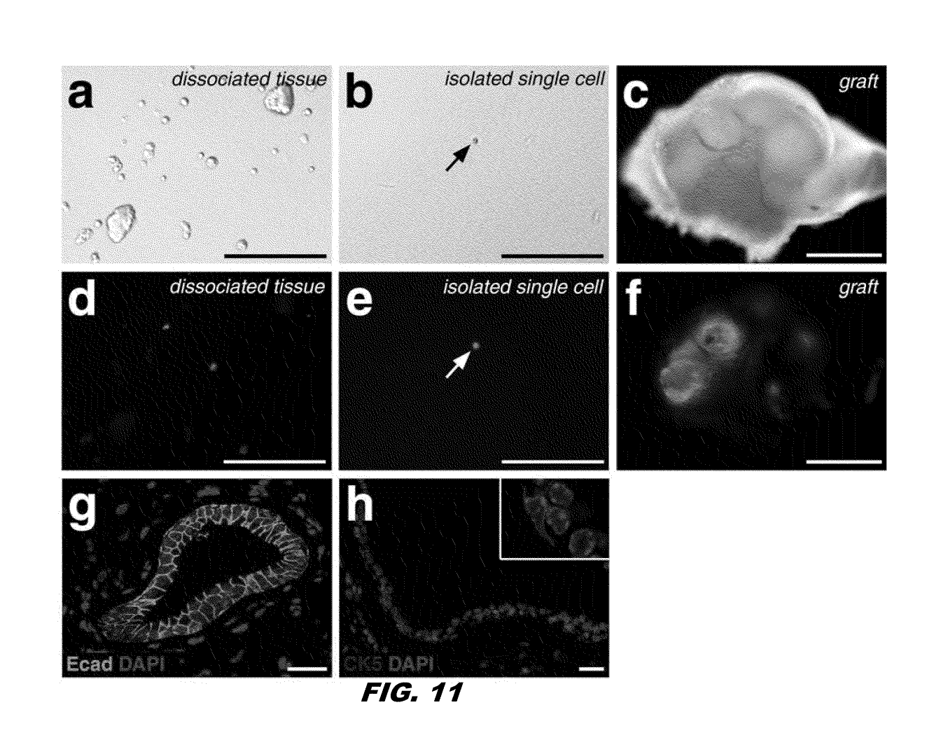

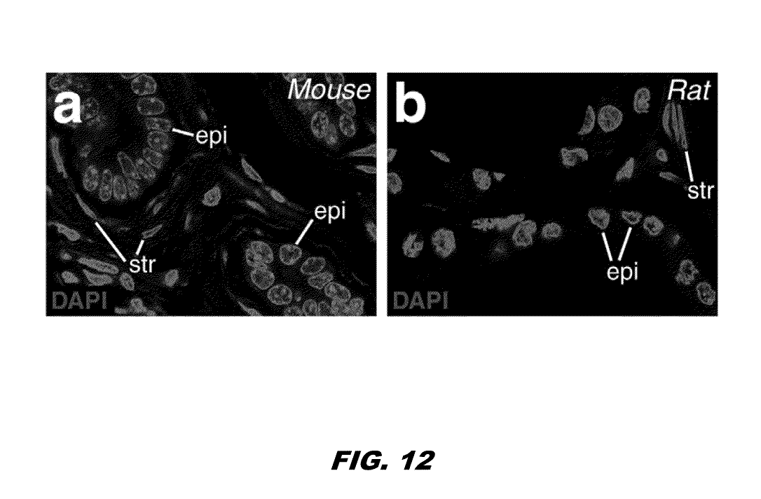

Next, we investigated whether CARNs could reconstitute prostate tissue in grafts generated from single or multiple lineage-marked CARNs (FIG. 3A; FIG. 10). To examine single lineage-marked CARNs, we isolated individual YFP⁺ cells from suspensions of dissociated prostate cells, followed by recombination with rat urogenital mesenchyme cells and renal grafting in immunodeficient male mice (FIG. 11A-F). The resulting grafts generated prostatic ducts with epithelial cells that were entirely YFP⁺ and that expressed luminal markers (E-cadherin, CK18, AR), basal markers (p63, CK5), or neuroendocrine markers (synaptophysin) (FIG. 3B-G; FIG. 11G, 11H); importantly, these ducts produced secretory proteins and expressed Nkx3.1, which is prostate-specific (FIG. 3C, 3I). Furthermore, we verified that the tissue formed in these grafts was unequivocally of mouse origin by nuclear morphology³¹ (FIG. 12). Notably, the frequency of successful single-cell transplantation of lineage-marked YFP⁺ cells (37%, n=43) was significantly greater than for the YFP⁻ control (3%, n=31; p<0.001) (FIG. 3J).

### Nkx3.1 Regulates Progenitor Maintenance

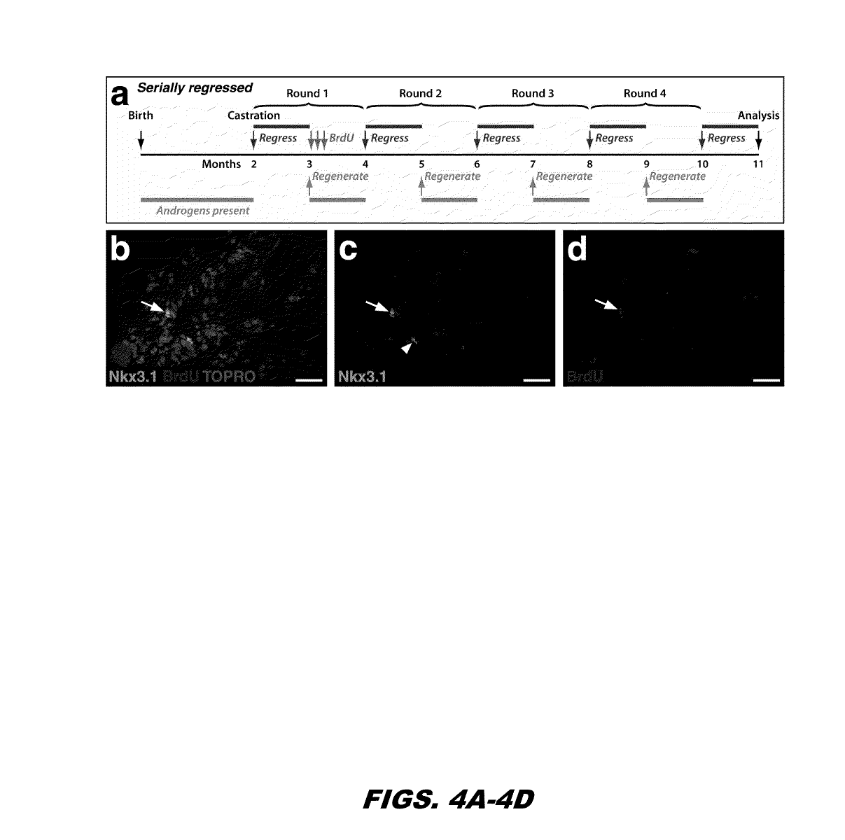

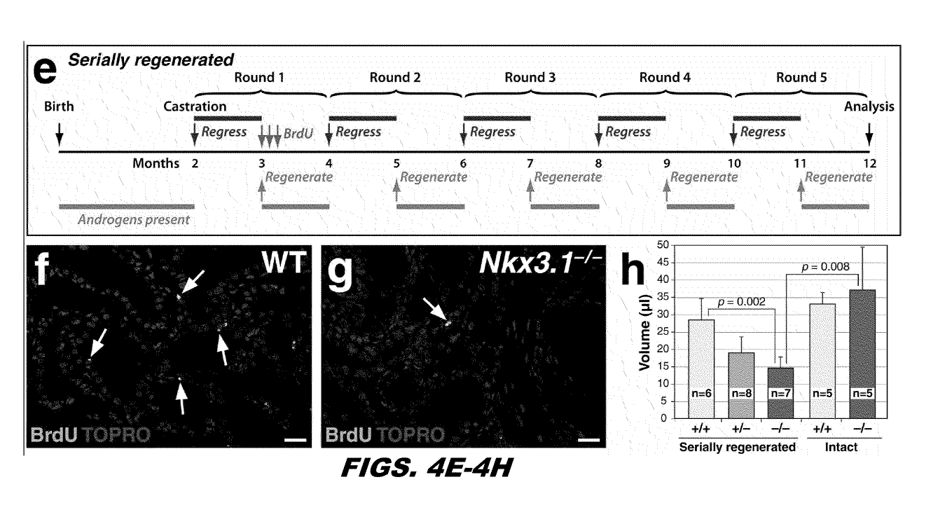

Since Nkx3.1 expression marks the CARNs population, we next investigated whether Nkx3.1 regulates progenitor maintenance and/or differentiation. First, we examined whether BrdU label-retaining cells (LRCs) might be affected by Nkx3.1 inactivation, since in many tissues (but not all³²) such long-term growth-quiescent cells are enriched for progenitors^{33,34}. In the prostate, such LRCs can be identified by BrdU pulse-chase labeling during serial regression/regeneration⁶ (FIG. 4A). Under conditions in which 1.4% of epithelial cells (n=33,086) retained BrdU-labeling at the fifth regression, 14.0% of CARNs (n=193) were also BrdU-positive (FIG. 4B-4D; Table 3), indicating that a significant proportion of CARNs are also LRCs. Secondly, the percentage of LRCs in Nkx3.1 mutants (0.3%, n=86,601) was significantly less than in wild-type controls (0.8%, n=75,758; p=0.003) after five rounds of regression/regeneration (FIG. 4E-4G; Table 3), suggesting a decrease in prostate epithelial progenitors.

TABLE 3
Quantitation of BrdU label-retaining cells.

| | | | |
|---|---|---|---|
| Mouse ID | # BrdU⁺ cells | # epithelial cells | % BrdU⁺ cells/total epithelial cells |

1. BrdU labeling efficiency immediately after labeling

| | | | |
|---|---|---|---|
| 0050 | 2,533 | 17,060 | 14.8% |
| 0057 | 1,822 | 15,851 | 11.5% |
| 0052 | 2,042 | 16,259 | 12.6% |
| 0053 | 720 | 16,123 | 4.5% |
| Totals | 7,117 | 65,293 | 10.9% |

2. Percentage of LRCs after five rounds of serial regression

| | | | |
|---|---|---|---|
| 0507 | 92 | 6,588 | 1.4% |
| 0502 | 151 | 15,388 | 1.0% |
| 2006 | 224 | 11,110 | 2.0% |
| Totals | 467 | 33,086 | 1.4% |

3. Overlap of LRCs with CARNs

| Mouse ID | # BrdU⁺Nkx3.1⁺ cells | # Nkx3.1⁺ cells | % BrdU⁺Nkx3.1⁺/Nkx3.1⁺ cells |
|---|---|---|---|
| 0507 | 6 | 42 | 14.3% |
| 0502 | 6 | 60 | 10.0% |
| 2006 | 15 | 91 | 16.5% |
| Totals | 27 | 193 | 14.0% |

4. Percentage of LRCs after five rounds of serial regeneration

| | | | |
|---|---|---|---|
| Mouse ID | # BrdU⁺ cells | # epithelial cells | % BrdU⁺ cells/total epithelial cells |

a. Wild-type

| | | | |
|---|---|---|---|
| 1011 | 239 | 28,971 | 0.82% |
| 4212 | 219 | 27,034 | 0.81% |
| 4782 | 156 | 19,753 | 0.79% |
| Totals | 614 | 75,758 | 0.81% |

b. Nkx3.1^{-/-} mutant

| | | | |
|---|---|---|---|
| 0909 | 36 | 24,602 | 0.15% |
| 0977 | 35 | 14,048 | 0.25% |
| 8371 | 89 | 15,394 | 0.58% |
| 8372 | 66 | 15,903 | 0.48% |
| 8373 | 46 | 16,654 | 0.28% |
| Totals | 272 | 86,601 | 0.31% (p = 0.003) |

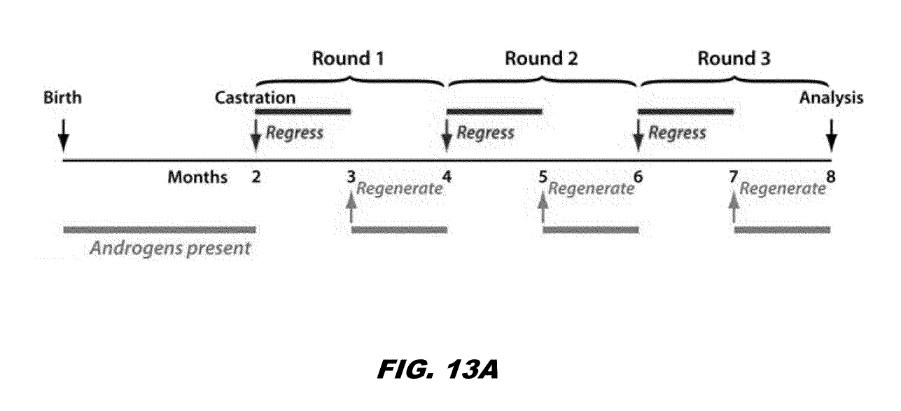

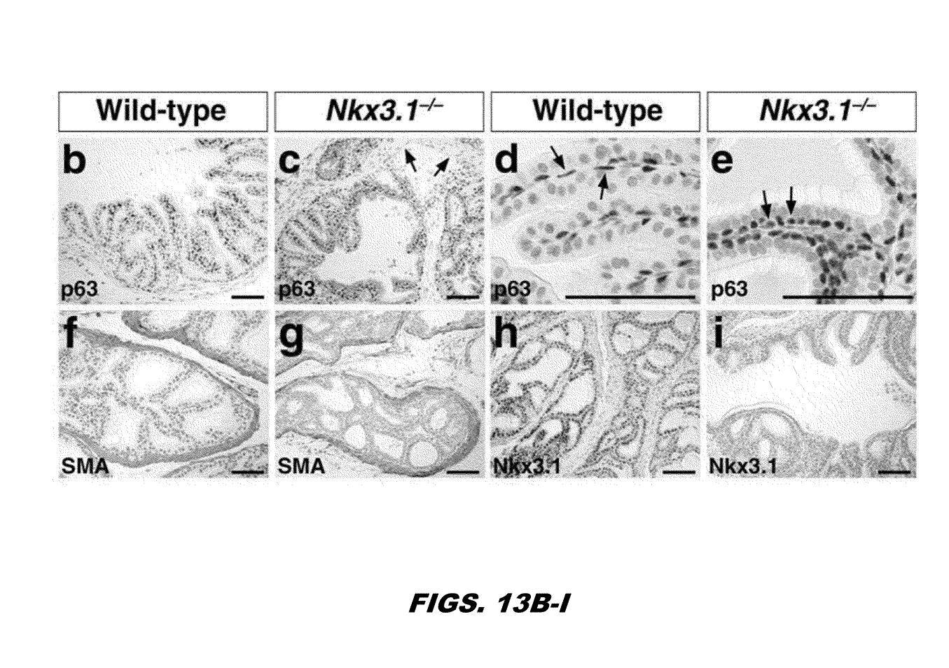

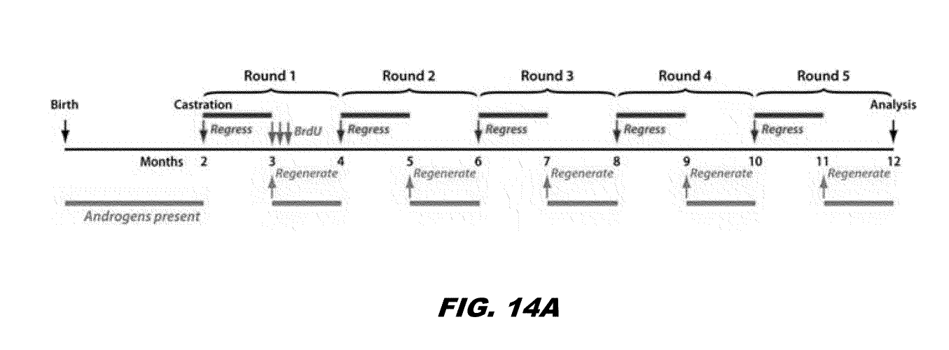

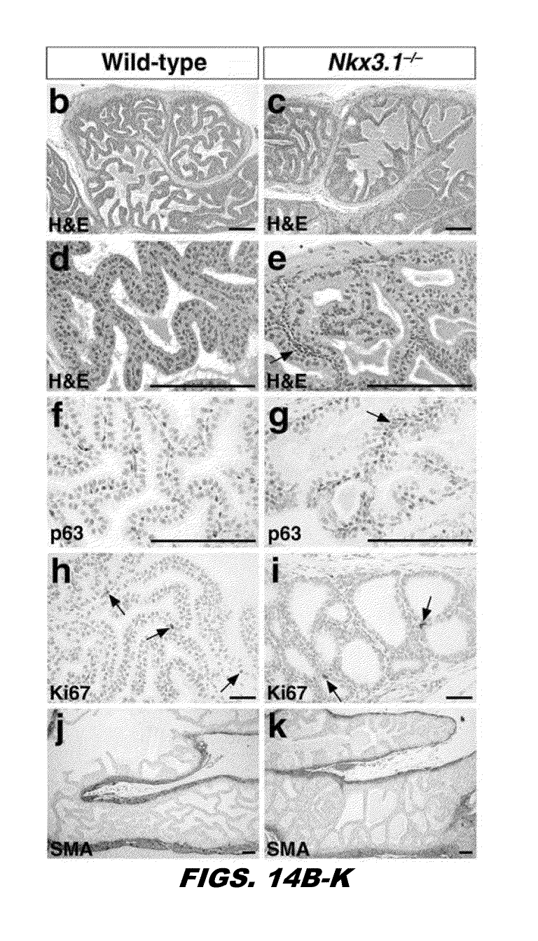

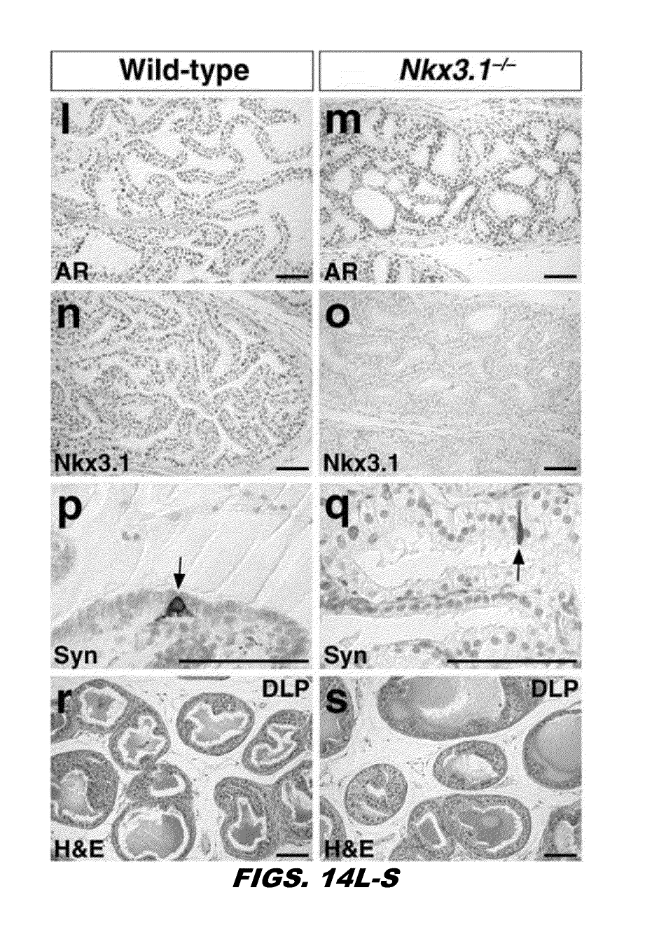

We also observed phenotypic alterations in Nkx3.1 mutants after five rounds of serial regeneration, including reduced anterior prostate volume relative to wild-type controls (FIG. 4H). At the histological level, the characteristic hyperplasia and PIN phenotype of Nkx3.1 homozygous (n=10) as well as heterozygous mice (n=8) was partially suppressed by three or five rounds of serial regeneration, while no abnormalities were observed in wild-type controls (n=9) treated in parallel (FIG. 13, FIG. 14; Table 4). Notably, the proliferative index of serially regenerated Nkx3.1^{-/-} mutants was similar to controls (FIG. 14H, 14I), in contrast with the elevated proliferation observed in intact Nkx3.1 homozygotes²¹. Overall, these findings suggest that Nkx3.1 is required for prostate stem cell maintenance during serial regression/regeneration.

TABLE 4
Summary of prostate phenotypes in control and Nkx3.1 mutant
mice after three and five rounds of serial regression/regeneration.

| Nkx3.1 genotype | Regeneration | Number | Normal | Hyperplasia | PIN |
|---|---|---|---|---|---|
| +/+ | 3 rounds | 7 | 4 | 2 | 1 |
| +/+ | 5 rounds | 9 | 8 | 1 | 0 |
| +/+ | Intact^{a} | 6 | 4 | 1 | 1 |
| +/− | 3 rounds | 8 | 3 | 5 | 0 |
| +/− | 5 rounds | 8 | 7 | 1 | 0 |
| +/− | Intact^{a} | 7 | 2 | 2 | 3 |
| −/− | 3 rounds | 5 | 0 | 5 | 0 |
| −/− | 5 rounds | 10 | 3 | 7 | 0^{b} |
| −/− | Intact^{a} | 9 | 3 | 1 | 5 |

Phenotypes were scored blind using hematoxylin-eosin stained anterior prostate sections, using standard criteria^{S1}.
^{a}Data for intact mice at 6-12 months of age have been previously published^{S2}.
^{b}Difference in frequency of PIN between serially regenerated and intact Nkx3.1 homozygous animals is significant (p < 0.01).

CARNs are a Cell of Origin for Cancer

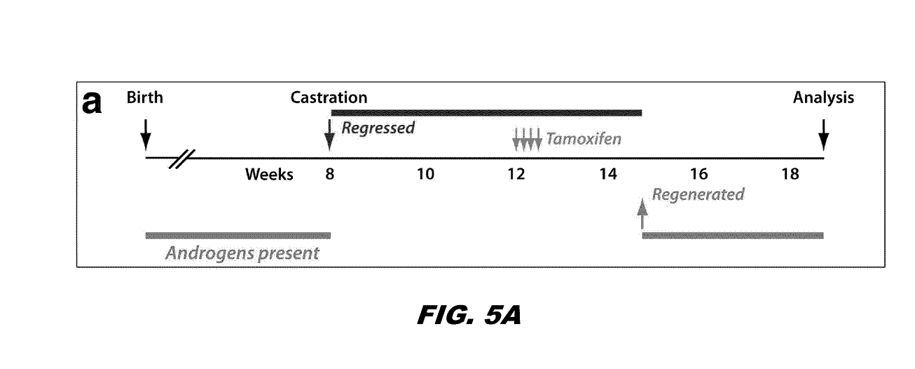

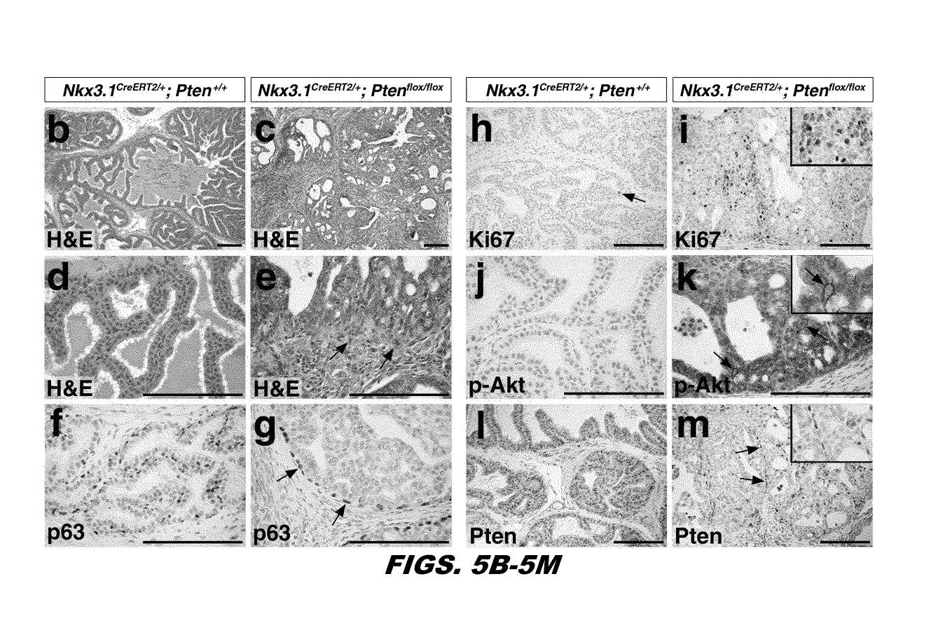

Finally, we investigated whether CARNs could represent a target of oncogenic transformation in prostate cancer, by examining the effects of CARNs-specific deletion of the tumor suppressor gene Pten, a key regulator of the PI3-kinase/Akt signaling pathway that is frequently inactivated in human prostate cancer. For this purpose, we inducibly deleted Pten in the CARNs population of castrated male mice carrying a conditional Pten allele³⁵ together with the inducible Nkx3.1^{CreERT2} allele (FIG. 5A). Following androgen-mediated regeneration of the prostate, we observed rapid formation of high-grade PIN and carcinoma with evidence of microinvasion in the Nkx3.1^{CreERT2/+}; pten^{flox/flox mice} (n=6), whereas control Nkx3.1^{CreERT2/+}; Pten^{+/+} mice were phenotypically normal (n=6) (FIG. 5B-5E). Notably, these PIN and carcinoma lesions showed increased proliferation and loss of basal cells, and displayed membrane-localized phospho-Akt activity (FIG. 5F-5M). These data indicate that the CARNs population can serve as a cell of origin for prostate cancer, and that the resulting carcinoma lesions have a luminal phenotype.

Discussion

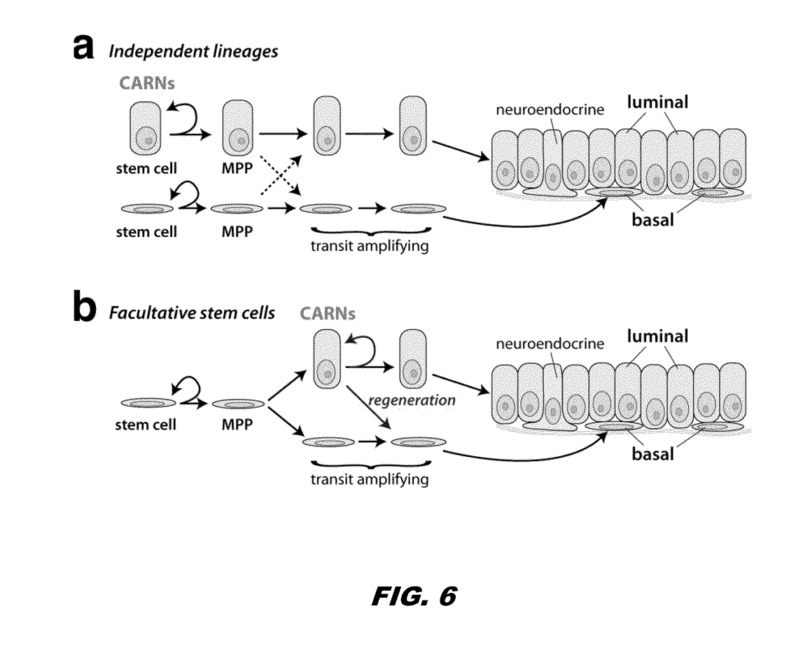

In context with previous studies describing basal stem cells^{9,10,13}, our identification of CARNs as luminal stem cells indicates the existence of distinct non-overlapping stem cell populations in the prostate epithelium. Consequently, we can propose two general models for the lineage relationship between CARNs and a basal stem cell population. One possibility is that basal and luminal cell types may possess independent progenitors that have partially redundant stem cell activities (FIG. 6A). A second possibility is that CARNs represent facultative or “potential” stem cells corresponding to transit-amplifying cells that acquire stem cell properties during regeneration/wound healing responses, as have been described in the mammalian testis and pancreas³⁶⁻³⁸ (FIG. 6B). In this model, the facultative stem cells that drive prostate regeneration could be independent from the stem cells for prostate organogenesis, and might co-exist in the adult gland; such a dual progenitor system functions during Drosophila tracheal remodeling³⁹.

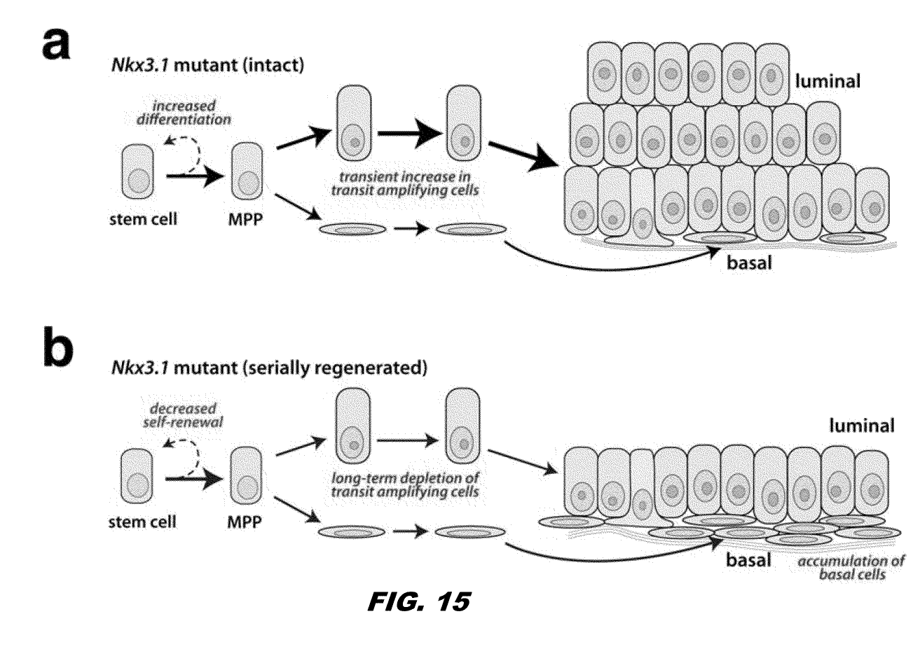

In addition, the observed defect in stem cell maintenance in Nkx3.1 mutants suggests a functional role for Nkx3.1 expression in CARNs. Thus, Nkx3.1 inactivation might result in increased differentiation of CARNs and expansion of a proliferative transit-amplifying population (FIG. 15). This interpretation is consistent with the finding that the duration of epithelial proliferation during prostate regeneration is prolonged in Nkx3.1 mutants relative to wild-type⁴⁰. The function of Nkx3.1 in stem cell maintenance may be direct, consistent with its feedback loop with androgen receptor and role in prostate epithelial differentiation^{20,41}, or may be indirect, for example due to increased oxidative damage with aging⁴².

Finally, the importance of the stem cell compartment as a target of oncogenic transformation has been highlighted by studies showing that stem cell populations in lung and colon are efficient cells of origin for cancer^{43,44}. In the case of prostate cancer, the identification of a castration-resistant stem cell population as a cell of origin also has implications for the onset of hormone-refractory disease. Thus, if oncogenic transformation of CARNs can result in the formation of a putative cancer stem cell, the eventual emergence of hormone-refractory disease may be pre-figured through an initiating event during prostate carcinogenesis.

Methods

Gene targeting and genotyping. Briefly, the Nkx3.1^{CreERT2/+} allele was generated by gene targeting using standard techniques; the Nkx3.1 null mutant mice have been previously described²¹. R26R-lacZ and Pten conditional mutant mice were obtained from the Jackson Laboratory Induced Mutant Resource; the R26R-YFP mice were provided by Dr. Frank Costantini. All lines were maintained on a hybrid C57BL/6-129/Sv strain background.

The Nkx3.1^{CreERT2/+} allele was generated by gene targeting using standard techniques⁴⁶. The targeting vector was generated using a 5′ arm corresponding to a 3.5 kb PCR fragment from a Nkx3.1 genomic clone²¹ up to the translation initiation site of Nkx3.1, and a 3′ arm corresponding to a 4.0 kb PCR fragment of genomic sequence (FIG. 8A). The positive selection cassette corresponded to the self-excising ACE-Cre/PolII-neo selection cassette from the pACN vector⁴⁷, while negative selection was provided by the PGK-tk cassette from the pPNT vector⁴⁸. Primers for generating the 5′ arm were: 5′-ACC GGA ATT CTC CGC TGC GCG CCG CTT TTG C-3′ [SEQ ID NO: 1] and 5′-ACC CAA GCT TCA TGC CTG CAG GTC GGA GGC C-3′ [SEQ ID NO: 2]. Primers to amplify the 3′ arm were: 5′-CTA GTC TAG AGC GGC TCA CCT CCT TCC TCA-3′ [SEQ ID NO: 3] and 5′-CTA GTC TAG AGG ATG GCA GGA GAG GTC ACT GC-3′ [SEQ ID NO: 4]. The gap between the 5′ and 3′ arms is approximately 80 bp, such that the 3′ arm contains the majority of exon 1 together with intron 1, exon 2, and 200 by of genomic sequence 3′ of the transcription termination site. The pGS-CreER^{T2} vector²⁸ was generously provided by Pierre Chambon, and was modified by insertion of a 65 by intron from ACE-Cre⁴⁷ between the PshAI and ClaI sites of the CreER^{T2} sequence. Culture and transfection of mouse ES cells followed standard protocols⁴⁶. Homologous recombinants in TC1 ES cells⁴⁹ were selected by positive-negative selection followed by Southern blot screening. 1/260 clones analyzed was properly targeted, and this clone was used to generate germline chimeras.

Mouse genotyping. Genotyping for the Nkx3.1^{CreRT2} allele was performed by Southern blotting or by PCR using tail genomic DNA. Primers for PCR genotyping were as follows: for the Nkx3.1 wild-type allele, 5′-CTC CGC TAC CCT AAG CAT CC-3′ [SEQ ID NO: 5] and 5′-GAC ACT GTC ATA TTA CTT GGA CC-3′ [SEQ ID NO: 6], which amplifies a region deleted in the targeting vector; and for the Nkx3.1^{CreERT2} allele, 5′-CAG ATG GCG CGG CAA CAC C-3′ [SEQ ID NO: 7] and 5′-GCG CGG TCT GGC AGT AAA AAC-3 [SEQ ID NO: 8]′.

The primers for genotyping Nkx3.1 mutant mice were: 5′-GCC AAC CTG CCT CAA TCA CTA AGG-3′ (wild-type Nkx3.1 forward [SEQ ID NO: 9]), 5′-TTC CAC ATA CAC TTC ATT CTC AGT-3′ (mutated forward [SEQ ID NO: 10]), and 5′-GCC AAC CTG CCT CAA TCA CTA AGG-3′ (wild-type and mutated reverse [SEQ ID NO: 11]). The primers for genotyping the R26R-lacZ Cre-reporter were: 5′-CCG CGC TGT ACT GGA GGC TGA AG-3′ (forward [SEQ ID NO: 12]) and 5′-ATA CTG CAC CGG GCG GGA AGG AT-3′ (reverse [SEQ ID NO: 13]). Primers for genotyping the Pten conditional (Pten^{flox}) allele were: 5′-ACT CAA GGC AGG GAT GAG C-3′ (forward [SEQ ID NO: 14]) and 5′-GTC ATC TTC ACT TAG CCA TTG G-3′ (reverse [SEQ ID NO: 15]). Primers for genotyping the R26R-YFP mice were: 5′-GCG AAG AGT TTG TCC TCA ACC-3′ (mutated forward [SEQ ID NO: 16]), 5′-GGA GCG GGA GAA ATG GAT ATG-3′ (wild-type forward [SEQ ID NO: 17]) and 5′-AAA GTC GCT CTG AGT TGT TAT-3′ (wild-type and mutated reverse [SEQ ID NO: 18]).

Mouse procedures. Briefly, castration of adult male mice was performed using standard techniques. For tamoxifen induction of Cre activity in mice containing Nkx3.1^{CreERT2/+}, mice were administered 9 mg/40 g tamoxifen for 4 consecutive days. For prostate regeneration, physiological levels of testosterone (1.875 μg/hr) were administered for four weeks by subcutaneous implantation of mini-osmotic pumps (Alzet)⁴⁵. When included, BrdU (100 mg/kg) was administered once daily during the first three days of regeneration. For single-cell transplantation, single YFP+ cells were isolated by mouth-pipetting under epifluorescence illumination from a dissociated prostate cell suspension obtained from castrated and tamoxifen-induced Nkx3.1^{CreERT2/+}, R26R-YFP/+ mice. A single YFP⁺ cell (or YFP⁻ cell as a control) was recombined with 2.5×10⁵ rat urogenital sinus mesenchyme cells in a 10 μl collagen pad, followed by transplantation under the kidney capsule of nude mice and harvesting after 10-12 weeks.

Castration of adult male mice was performed using standard techniques⁵⁰. Following castration at 8 weeks of age, mice were allowed to regress for four weeks to reach the fully involuted state. For tamoxifen induction of Cre activity in mice containing the Nkx3.1^{CreERT2} allele, mice were administered 9 mg/40 g tamoxifen (Sigma) suspended in corn oil, or vehicle alone for negative controls, by i.p. injection or oral gavage once daily for 4 consecutive days, followed by a chase period of 14 days.

For prostate regeneration, testosterone (Sigma) was dissolved at 25 mg/ml in 100% ethanol and diluted in PEG-400 to a final concentration of 7.5 mg/ml. Testosterone was administered for four weeks at a rate of 1.875 μg per hour delivered by subcutaneous implantation of mini-osmotic pumps (Alzet); this regimen yields physiological levels of serum testosterone⁴⁵. When included, BrdU (100 mg/kg) (Sigma) was also administered by i.p. injection once daily during the first three days of regeneration to label proliferating cells. After regeneration of the prostate, mice could be euthanized for analysis, or deprived of androgens by pump removal, returning to the regressed state after four additional weeks. At this point, mice were either euthanized for analysis, or osmotic pumps could be reimplanted for additional rounds of serial regression/regeneration.

For tissue recombination and renal grafting, prostate tissues (corresponding to the combined anterior, dorsolateral, and ventral lobes) were dissected and minced to small clumps, followed by enzymatic dissociation with 0.2% collagenase I (Invitrogen) in DMEM media with 10% fetal bovine serum for 90 min. Dissociated tissue was passed sequentially through 21, 23 and 26 gauge needles followed by a 40 μm cell strainer to obtain single-cell suspensions. The resulting cells were assessed for viability by trypan blue exclusion and counted. For grafts containing large numbers of epithelial cells, as in FIG. 10, 2.5×10⁵ dissociated prostate cells obtained from castrated and tamoxifen-induced Nkx3.1^{CreERT2/+}; R26R-YFP/+ mice were mixed with 2.5×10⁵ dissociated urogenital sinus mesenchyme (UGM) cells from E18.0 rat embryos. UGM cells were obtained from dissected urogenital sinus that was treated for 30 min in 1% trypsin, followed by mechanical dissociation and treatment with 0.1% collagenase B (Roche) for 30 min at 37°, and washing in PBS. Pelleted cell mixtures were resuspended in 10 μl of 1:5 collagen:setting buffer (10× Earle's Balanced Salt Solution (Life Technologies), 0.2 M NaHCO₃, 50 mM NaOH), and gelatinized in 37° C. for 20 minutes. Tissue recombinants were cultured in DMEM media with 10% fetal bovine serum supplemented with 10⁻⁷M dihydrotestosterone (DHT) overnight, followed by transplantation under the kidney capsules of nude mice. Grafts were harvested after 4-8 weeks of growth for analysis.

For single-cell grafts, as in FIG. 3, a single YFP⁺ (or YFP cell as a control) was isolated from the dissociated cell suspension from castrated and tamoxifen-induced Nkx3.1^{CreERT2/+}; R26R-YFP/+ prostates by mouth-pipetting under epifluorescence illumination on an Olympus IX51 inverted microscope with DP71 camera. This single cell was then recombined with 2.5×10⁵ dissociated urogenital sinus mesenchyme cells obtained from E18.0 rat embryos, and cultured and grafted as above. Grafts were harvested after 10-12 weeks of growth for analysis, and imaged under epifluorescence on an Olympus SZX16 stereomicroscope with DP71 camera. The resulting graft tissue was analyzed for YFP and other marker expression as described below, and counterstained with DAPI for visualization of nuclear morphology at high-power to distinguish mouse from rat nuclei³¹.

Grafts recovered from transplantation of a single lineage-marked YFP⁺ cell (n=16/43, 37%) were confirmed to be of mouse origin by YFP expression and nuclear morphology (FIG. 12), while the single graft (n=1/31, 3%) arising from a YFP⁻ cell was confirmed to be of mouse origin by nuclear morphology. Generation of prostatic ducts by a YFP cell might result from a CARN that was not lineage-marked by tamoxifen-induction, which is inefficient, or alternatively from a distinct stem cell type in the prostate epithelium. We note that a significant percentage (n=13/74, 18%) of the grafts contained ducts of rat origin, which are not included in FIG. 3J. These rat ducts likely arise from rat urogenital epithelial cells that are difficult to completely dissociate from the urogenital mesenchyme used in the graft, and can populate the graft under conditions in which the epithelial contribution is limiting.

For histological and immunofluorescence analysis, individual prostate lobes or renal grafts were dissected, and then fixed in 4% paraformaldehyde for subsequent cryoembedding in OCT compound (Sakura), or fixed in 10% formalin followed by paraffin embedding. Volume of dissected anterior prostate lobes was determined by physical displacement of known volumes of PBS solution in 0.5 ml centrifuge tubes.

Histology and immunostaining. Briefly, cryosections were stained with primary antibodies as listed in Table 5, and counterstained with TOPRO3 or DAPI (Invitrogen/Molecular Probes). Secondary antibodies were labeled with Alexa Fluor 488, 555, or 594 (Invitrogen/Molecular Probes). Immunofluorescence staining was imaged using a Leica TCS5 spectral confocal microscope. Cell counting was performed manually using confocal photomicrographs with at least three animals for each experiment or genotype analyzed.

TABLE 5
Antibodies used in this study.

| Antigen | Supplier | Ig type | Dilution |
|---|---|---|---|
| Androgen receptor | Sigma #A9853 | rabbit IgG | 1:500 |
| Androgen receptor (AN1-15) | Affinity Bioreagents MA1-150 | rat IgG | 1:50 |
| β-catenin | BD Biosciences #610153 | mouse IgG1 | 1:1000 |
| β-galactosidase | Rockland #100-4136 | rabbit IgG | 1:1000 |
| BrdU | Abcam #ab1893 | sheep IgG | 1:200 |
| CK5 | Covance #PRB-160P | rabbit IgG | 1:500 |
| CK14 | Biogenex #MU146-UC | mouse IgG1 | 1:100 |
| CK18 | Abcam #ab668 | mouse IgG1 | 1:100 |
| CD117 (ACK2) | eBioscience #14-1172 | rat IgG2b | 1:100 |
| CD117 (ACK45) | BD Pharmingen #553868 | rat IgG2b | 1:100 |
| Cre | Covance #PRB-106C | rabbit IgG | 1:1000 |
| E-cadherin | Cell Signaling #4046 | rabbit IgG | 1:200 |
| GFP | Invitrogen #A11122 | rabbit IgG | 1:1000 |
| GFP | Roche #11814460001 | mouse IgG1 | 1:200 |
| GFP | Abeam #ab13970 | chick IgY | 1:1000 |
| Ki67 | DakoCytomation #M7249 | rat IgG2a | 1:600 |
| Ki67 | Novocastra #NCL-Ki67p | rabbit IgG | 1:1400 |
| Nkx3.1 | Ref. S3 | rabbit IgG | 1:1000 or 1:8000 |
| Nkx3.1 | Ref. S4 | rabbit IgG | 1:2000 |
| p63 | Santa Cruz #sc-8431 | mouse IgG1 | 1:600 |
| p63 | Santa Cruz #sc-8343 | rabbit IgG | 1:50 |
| PTEN | Cell Signaling #9559 | rabbit IgG | 1:100 |
| Phospho-Akt | Cell Signaling #3787 | rabbit IgG | 1:50 |
| Smooth muscle actin | Sigma #A2547 | mouse IgG1 | 1:300 |
| Synaptophysin | Zymed #18-0130 | rabbit IgG | 1:500 |

Hematoxylin-eosin staining was performed using standard protocols on 6 μm paraffin sections. β-galactosidase staining was performed using 12 micron cryosections, which were incubated in staining solution (0.1 M PBS, 1.3 mM MgCl₂, 1 mg/ml X-gal, 0.02% Nonidet P-40, 5 mM K₄Fe(CN)₆, 5 mM K₃Fe(CN)₆, and 0.01% Na-deoxycholate) for 3 hours or overnight, followed by fixation in 10% formalin for 2 to 5 hours. Direct visualization of YFP was performed after washing 10 μm cryosections in PBST (PBS with 0.1% Triton X-100) 3 times, incubation with TOPRO3 (1:1000 diluted in PBST) (Invitrogen/Molecular Probes) for 30 min, and mounting with VECTASHIELD mounting medium (Vector Labs), which contains DAPI.

For immunohistochemical staining, 6 μm paraffin sections were deparaffinized in xylene, followed by antigen retrieval through boiling in antigen unmasking solution (Vector Labs). Slides were blocked in 10% normal serum or with blocking reagents provided in the M.O.M. kit (Vector Labs) for mouse primary antibodies, then incubated with primary antibodies overnight at 4° C. or room temperature. Primary antibodies and dilutions utilized are listed in Table 5. Secondary antibodies were obtained from Vectastain ABC kits (Vector Labs) and diluted 1:250 or 1:500. The signal was enhanced using the Vectastain ABC system and visualized with the NovaRed Substrate Kit (Vector Labs). The slides were counterstained with Harris Modified Hematoxylin (1:4 diluted in H₂O) (Fisher Scientific) and mounted with Clearmount (American Master*Tech Scientific). Immunohistochemical staining was imaged using a Nikon Eclipse E800 microscope equipped with a Nikon DXM1200 digital camera.

Immunofluorescence staining was performed on either 6 micron paraffin sections or 10 micron cryosections, which were incubated in 3% H₂O₂ and Antigen Unmasking Solution (Vector Labs). Primary antibodies and dilutions utilized are listed in Table 5. Slides were incubated with 10% normal goat (Vector Labs) or donkey serum (Sigma) and with primary antibodies diluted in the 10% normal goat or donkey serum overnight at 4° C. or room temperature. Slides then were incubated with secondary antibodies (diluted 1:500 in PBST) labeled with Alexa Fluor 488, 555, or 594 (Invitrogen/Molecular Probes). Detection of Nkx3.1, GFP, and Cre was enhanced using tyramide amplification (Invitrogen/Molecular Probes) by incubation of slides with HRP-conjugated secondary antibody (1:100 dilution) (Invitrogen/Molecular Probes), followed by incubation with tyramide 488 or 555 for 6 min. Sections were counterstained with TOPRO3 or TOTO3 (diluted 1:1000 in PBST) (Invitrogen/Molecular Probes) to visualize nuclei, and mounted with VECTASHIELD mounting medium (Vector Labs), which contains DAPI. Immunofluorescence staining was imaged using a Leica TCS5 spectral confocal microscope.

Quantitation and statistics. To calculate the number of CARNs in the regressed mouse prostate, we determined that there are an average of 112,000 total cells (n=5 animals; all lobes combined), of which 59% are epithelial as determined by immunoreactivity for the panepithelial marker CD24 (ref.⁹). Since 0.7% of epithelial cells in the regressed prostate are CARNs, there are approximately 460 CARNs in the total prostate. To determine the number of lineage-marked cells in the regressed prostate, we visualized 320 live YFP⁺ cells in dissociated prostate tissue (all lobes combined) from 5 castrated lineage-marked Nkx3.1^{CreERT2/+}; R26R-YFP/+ mice, for a total of 64 YFP⁺ live cells/mouse. For the experiment in FIG. 10, we performed 2 recombinations from these dissociated prostate cells, so that there were approximately 160 live YFP⁺ cells used in each graft.

For immunostaining experiments, cell numbers were counted manually using confocal 40× and 63× photomicrographs. Statistical analyses were performed using a two-sample T-test, X² test, or Fisher's Exact test as appropriate. At least three animals for each experiment or genotype were analyzed.

### REFERENCES

¹Abate-Shen, C. & Shen, M. M., Molecular genetics of prostate cancer. Genes Dev. 14, 2410-2434 (2000).

²English, H. F., Santen, R. J., & Isaacs, J. T., Response of glandular versus basal rat ventral prostatic epithelial cells to androgen withdrawal and replacement. Prostate 11 (3), 229-242 (1987).

³Evans, G. S. & Chandler, J. A., Cell proliferation studies in the rat prostate: II. The effects of castration and androgen-induced regeneration upon basal and secretory cell proliferation. Prostate 11 (4), 339-351 (1987).

⁴Sugimura, Y., Cunha, G. R., & Donjacour, A. A., Morphological and histological study of castration-induced degeneration and androgen-induced regeneration in the mouse prostate. Biol Reprod 34 (5), 973-983 (1986).

⁵Isaacs, J. T., Control of cell proliferation and cell death in the normal and neoplastic prostate: a stem cell model in Benign Prostatic Hyperplasia, edited by C. H. Rodgers et al. (Department of Helath and Human Services, Washington, D.C., 1985), pp. 85-94.

⁶Tsujimura, A. et al., Proximal location of mouse prostate epithelial stem cells: a model of prostatic homeostasis. J Cell Biol 157 (7), 1257-1265 (2002).

⁷Lawson, D. A. & Witte, O. N., Stem cells in prostate cancer initiation and progression. J Clin Invest 117 (8), 2044-2050 (2007).

⁸Senoo, M., Pinto, F., Crum, C. P., & McKeon, F., p63 is essential for the proliferative potential of stem cells in stratified epithelia. Cell 129 (3), 523-536 (2007).

⁹Lawson, D. A., Xin, L., Lukacs, R. U., Cheng, D., & Witte, O. N., Isolation and functional characterization of murine prostate stem cells. Proc Natl Acad Sci USA 104 (1), 181-186 (2007).

¹⁰Richardson, G. D. et al., CD133, a novel marker for human prostatic epithelial stem cells. J Cell Sci 117 (Pt 16), 3539-3545 (2004).

¹¹Burger, P. E. et al., Sca-1 expression identifies stem cells in the proximal region of prostatic ducts with high capacity to reconstitute prostatic tissue. Proc Natl Acad Sci USA 102 (20), 7180-7185 (2005).

¹²Xin, L., Lawson, D. A., & Witte, O. N., The Sca-1 cell surface marker enriches for a prostate-regenerating cell subpopulation that can initiate prostate tumorigenesis. Proc Natl Acad Sci USA 102 (19), 6942-6947 (2005).

¹³Goldstein, A. S. et al., Trop2 identifies a subpopulation of murine and human prostate basal cells with stem cell characteristics. Proc Natl Acad Sci USA 105 (52), 20882-20887 (2008).

¹⁴Leong, K. G., Wang, B. E., Johnson, L., & Gao, W. Q., Generation of a prostate from a single adult stem cell. Nature 456 (7223), 804-808 (2008).

¹⁵Kurita, T., Medina, R. T., Mills, A. A., & Cunha, G. R., Role of p63 and basal cells in the prostate. Development 131 (20), 4955-4964 (2004).

¹⁶Kasper, S., Stem cells: The root of prostate cancer? J Cell Physiol 216, 332-336 (2008).

¹⁷Wang, S. et al., Pten deletion leads to the expansion of a prostatic stem/progenitor cell subpopulation and tumor initiation. Proc Natl Acad Sci USA 103 (5), 1480-1485 (2006).

¹⁸ Grisanzio, C. & Signoretti, S., p63 in prostate biology and pathology. J Cell Biochem 103 (5), 1354-1368 (2008).

¹⁹Humphrey, P. A., Diagnosis of adenocarcinoma in prostate needle biopsy tissue. J Clin Pathol 60 (1), 35-42 (2007).

²⁰Abate-Shen, C., Shen, M. M., & Gelmann, E., Integrating differentiation and cancer: the Nkx3.1 homeobox gene in prostate organogenesis and carcinogenesis. Differentiation 76 (6), 717-727 (2008).

²¹Bhatia-Gaur, R. et al., Roles for Nkx3.1 in prostate development and cancer. Genes Dev. 13 (8), 966-977 (1999).

²²Abdulkadir, S. A. et al., Conditional loss of Nkx3.1 in adult mice induces prostatic intraepithelial neoplasia. Mol. Cell. Biol. 22 (5), 1495-1503 (2002).

²³Kim, M. J. et al., Nkx3.1 mutant mice recapitulate early stages of prostate carcinogenesis. Cancer Res. 62 (11), 2999-3004 (2002).

²⁴Chen, H., Mutton, L. N., Prins, G. S., & Bieberich, C. J., Distinct regulatory elements mediate the dynamic expression pattern of Nkx3.1. Dev Dyn 234 (4), 961-973 (2005).

²⁵Sciavolino, P. J. et al., Tissue-specific expression of murine Nkx3.1 in the male urogenital system. Dev. Dyn. 209, 127-138 (1997).

²⁶Bieberich, C. J., Fujita, K., He, W. W., & Jay, G., Prostate-specific and androgen-dependent expression of a novel homeobox gene. J. Biol. Chem. 271 (50), 31779-31782 (1996).

²⁷Feil, R., Wagner, J., Metzger, D., & Chambon, P., Regulation of Cre recombinase activity by mutated estrogen receptor ligand-binding domains. Biochem Biophys Res Commun 237 (3), 752-757 (1997).

²⁸Indra, A. K. et al., Temporally-controlled site-specific mutagenesis in the basal layer of the epidermis: comparison of the recombinase activity of the tamoxifen-inducible Cre-ER(T) and Cre-ER(T2) recombinases. Nucleic Acids Res 27 (22), 4324-4327 (1999).

²⁹Srinivas, S. et al., Cre reporter strains produced by targeted insertion of EYFP and ECFP into the ROSA26 locus. BMC Dev Biol 1, 4 (2001).

³⁰Soriano, P., Generalized lacZ expression with the ROSA26 Cre reporter strain. Nat. Genet. 21 (1), 70-71 (1999).

³¹Cunha, G. R. & Vanderslice, K. D., Identification in histological sections of species origin of cells from mouse, rat and human. Stain Technol 59 (1), 7-12 (1984).

³²Kiel, M. J. et al., Haematopoietic stem cells do not asymmetrically segregate chromosomes or retain BrdU. Nature 449 (7159), 238-242 (2007).

³³Bickenbach, J. R. & Holbrook, K. A., Label-retaining cells in human embryonic and fetal epidermis. J Invest Dermatol 88 (1), 42-46 (1987).

³⁴Cotsarelis, G., Cheng, S. Z., Dong, G., Sun, T. T., & Lavker, R. M., Existence of slow-cycling limbal epithelial basal cells that can be preferentially stimulated to proliferate: implications on epithelial stem cells. Cell 57 (2), 201-209 (1989).

³⁵Groszer, M. et al., Negative regulation of neural stem/progenitor cell proliferation by the Pten tumor suppressor gene in vivo. Science 294 (5549), 2186-2189 (2001).

³⁶Nakagawa, T., Nabeshima, Y., & Yoshida, S., Functional identification of the actual and potential stem cell compartments in mouse spermatogenesis. Dev Cell 12 (2), 195-206 (2007).

³⁷Xu, X. et al., Beta cells can be generated from endogenous progenitors in injured adult mouse pancreas. Cell 132 (2), 197-207 (2008).

³⁸Barroca, V. et al., Mouse differentiating spermatogonia can generate germinal stem cells in vivo. Nat Cell Biol 11 (2), 190-196 (2009).

³⁹Weaver, M. & Krasnow, M. A., Dual origin of tissue-specific progenitor cells in Drosophila tracheal remodeling. Science 321 (5895), 1496-1499 (2008).

⁴⁰Magee, J. A., Abdulkadir, S. A., & Milbrandt, J., Haploinsufficiency at the Nkx3.1 locus. A paradigm for stochastic, dosage-sensitive gene regulation during tumor initiation. Cancer Cell 3 (3), 273-283 (2003).

⁴¹Lei, Q. et al., NKX3.1 stabilizes p53, inhibits AKT activation, and blocks prostate cancer initiation caused by PTEN loss. Cancer Cell 9 (5), 367-378 (2006).

⁴²Ouyang, X., DeWeese, T. L., Nelson, W. G., & Abate-Shen, C., Loss-of-function of Nkx3.1 promotes increased oxidative damage in prostate carcinogenesis. Cancer Res 65 (15), 6773-6779 (2005).

⁴³Kim, C. F. et al., Identification of bronchioalveolar stem cells in normal lung and lung cancer. Cell 121 (6), 823-835 (2005).

⁴⁴Barker, N. et al., Crypt stem cells as the cells-of-origin of intestinal cancer. Nature 457 (7229), 608-611 (2009).

⁴⁵Banach-Petrosky, W. et al., Prolonged exposure to reduced levels of androgen accelerates prostate cancer progression in Nkx3.1; Pten mutant mice. Cancer Res 67 (19), 9089-9096 (2007).

⁴⁶Nagy, A., Gertsenstein, M., Vintersten, K., & Behringer, R., Manipulating the Mouse Embryo: A Laboratory Manual. (Cold Spring Harbor Laboratory Press, Cold Spring Harbor, N.Y., 2003).

⁴⁷Bunting, M., Bernstein, K. E., Greer, J. M., Capecchi, M. R., & Thomas, K. R., Targeting genes for self-excision in the germ line. Genes Dev. 13 (12), 1524-1528 (1999).

⁴⁸Tybulewicz, V. L., Crawford, C. E., Jackson, P. K., Bronson, R. T., & Mulligan, R. C., Neonatal lethality and lymphopenia in mice with a homozygous disruption of the c-abl proto-oncogene. Cell 65 (7), 1153-1163 (1991).

⁴⁹Deng, C., Wynshaw-Boris, A., Zhou, F., Kuo, A., & Leder, P., Fibroblast growth factor receptor 3 is a negative regulator of bone growth. Cell 84, 911-921 (1996).

⁵⁰Gao, H., Ouyang, X., Banach-Petrosky, W. A., Shen, M. M., & Abate-Shen, C., Emergence of androgen independence at early stages of prostate cancer progression in nkx3.1; pten mice. Cancer Res 66 (16), 7929-7933 (2006).

### ADDITIONAL REFERENCES FOR SUPPLEMENTARY MATERIAL

^{S1}Park, J. H. et al., Prostatic intraepithelial neoplasia in genetically engineered mice. Am J Pathol 161 (2), 727-735 (2002).

^{S2}Kim, M. J. et al., Nkx3.1 mutant mice recapitulate early stages of prostate carcinogenesis. Cancer Res. 62 (11), 2999-3004 (2002).

^{S3}Kim, M. J. et al., Cooperativity of Nkx3.1 and Pten loss of function in a mouse model of prostate carcinogenesis. Proc. Natl. Acad. Sci. USA 99 (5), 2884-2889 (2002).

^{S4}Chen, H., Mutton, L. N., Prins, G. S., & Bieberich, C. J., Distinct regulatory elements mediate the dynamic expression pattern of Nkx3.1. Dev Dyn 234 (4), 961-973 (2005).

^{S5}Bunting, M., Bernstein, K. E., Greer, J. M., Capecchi, M. R., & Thomas, K. R., Targeting genes for self-excision in the germ line. Genes Dev. 13 (12), 1524-1528 (1999).

^{S6}Cunha, G. R. & Vanderslice, K. D., Identification in histological sections of species origin of cells from mouse, rat and human. Stain Technol 59 (1), 7-12 (1984).

^{S7}Lei, Q. et al., NKX3.1 stabilizes p53, inhibits AKT activation, and blocks prostate cancer initiation caused by PTEN loss. Cancer Cell 9 (5), 367-378 (2006).

What is claimed:

1. An isolated population of luminal stem cells obtained from the prostate epithelium that express Nkx3.1 in the absence of androgens.

2. A population of luminal stem cells from the prostate epithelium isolated by a single-cell transplantation assay, wherein the cells express Nkx3.1 in the absence of androgens.

3. The population of luminal stem cells of claim 1 or 2, wherein the cells are castration-resistant.

4. The population of luminal stem cells of claim 1 or 2, wherein the cells further express cytokeratin 8 (CK8), cytokeratin 18 (CK18), Androgen receptor (AR), or a combination thereof.

5. The population of luminal stem cells of claim 1 or 2, wherein the cells fail to express the Ki67 marker.

6. The population of luminal stem cells of claim 1 or 2, wherein the cells are obtained from the anterior region of the mouse prostate.

7. The population of luminal stem cells of claim 1, wherein the population is obtained through a single-cell transplantation assay.

8. An isolated cell of origin for prostate cancer, wherein the cell expresses Nkx3.1 in the absence of androgens.

9. The isolated cell of origin of claim 8, wherein the cell further expresses cytokeratin 8 (CK8), cytokeratin 18 (CK18), Androgen receptor (AR), or a combination thereof.

10. The isolated cell of origin of claim 8, wherein the cell fails to express the Ki67 marker.

11. A purified preparation of prostate epithelium luminal stem cells wherein the cells express Nkx3.1 in the absence of androgens and cytokeratin 8 (CK8), cytokeratin 18 (CK18), Androgen receptor (AR), or a combination thereof.

12. A purified preparation of prostate epithelium luminal stem cells wherein the cells express Nkx3.1 in the absence of androgens and cytokeratin 8 (CK8), cytokeratin 18 (CK18), Androgen receptor (AR), or a combination thereof, and do not express Ki67.

13. A method for diagnosing whether a patient is at risk of developing prostate cancer, the method comprising:
(a) obtaining a tissue, a tissue sample, or a cell population;
(b) contacting the tissue, the tissue sample, or the cell population with an agent that binds to Nkx3.1; and
(c) determining whether the agent has bound to the tissue, the tissue sample, or the cell population,
wherein binding indicates the presence of stem cells that express Nkx3.1.

14. A method for diagnosing whether a subject is at risk of developing prostate cancer, the method comprising:
(a) obtaining a biological sample from a subject; and
(b) determining whether or not stem cells that express Nkx3.1 are present in a biological sample from the subject as compared to a non-prostate cancer subject.

15. A method for diagnosing prostate cancer stem cells in metastatic cells or metastases, the method comprising:
(a) obtaining a tissue, a tissue sample, or a cell population;
(b) contacting the tissue, the tissue sample, or the cell population with an agent that binds to Nkx3.1; and
(c) determining whether the agent has bound to the tissue, the tissue sample, or the cell population, wherein binding indicates the presence of stem cells that express Nkx3.1.

16. A method for diagnosing prostate cancer stem cells in metastatic cells or metastases, the method comprising:
(a) obtaining a biological sample from a subject; and
(b) determining whether or not stem cells that express Nkx3.1 are present in a biological sample from the subject as compared to a non-prostate cancer subject.

17. The method of claim 13, 14, 15, or 16, wherein the step of determining is performed using a method selected from the group consisting of RT PCR, in situ hybridization, Northern blotting RNAase protection, or any combination thereof.

18. The method of claim 13, 14, 15, or 16, wherein the tissue, the tissue sample, or the cell population comprises prostate tissue or cells, bone marrow, peripheral blood, lymph nodes, tumor metastases, or a combination thereof.

19. The method of claim 13, 14, 15, or 16, wherein the stem cells are castration-resistant, luminal prostate stem cells.

20. The method of claim 13 or 15, wherein the agent is an antibody.

21. The method of claim 20, wherein the antibody is a polyclonal antibody or a monoclonal antibody.

22. The method of claim 19, wherein the castration-resistant, luminal prostate stem cells are human stem cells or mouse stem cells.

23. A diagnostic kit for detecting the presence of Nkx3.1 in a sample, the kit comprising a nucleic acid molecule that specifically hybridizes to or a primer combination that amplifies a Nkx3.1 nucleic acid sequence.

24. A diagnostic kit for determining whether a sample from a subject exhibits a presence of prostate cancer or a predisposition to developing prostate cancer, the kit comprising a nucleic acid primer that specifically hybridizes to a luminal prostate cancer biomarker, wherein the primer will prime a polymerase reaction only when a luminal prostate cancer biomarker is present.

25. The kit of claim 23, wherein the nucleic acid molecule comprises a nucleic acid primer or nucleic acid probe.

26. The kit of claim 23, wherein the Nkx3.1 nucleic acid sequence comprises at least about 90% of SEQ ID NO: 20.

27. The kit of claim 25, wherein the probe comprises at least 10 consecutive nucleotide bases comprising SEQ ID NO: 20.

28. The kit of claim 25, wherein the probe comprises a reverse complement of at least 10 consecutive nucleotide bases comprising SEQ ID NO: 20.

29. The kit of claim 23 or 25, wherein the primer comprises a nucleotide sequence comprises SEQ ID NOS: 9, 11 or a combination thereof.

30. The kit of claim 24, wherein the luminal prostate cancer biomarker is Nkx3.1.

31. The kit of claim 23 or 24, wherein the sample is from a human or non-human animal.

32. The kit of claim 23 or 24, wherein the sample comprises prostate tissue or cells, bone marrow, peripheral blood, lymph nodes, tumor metastases, or a combination thereof.

33. A method for reconstituting prostate tissue, the method comprising:
(a) isolating luminal stem cells expressing Nkx3.1 in the absence of androgens from dissociated prostate cells of a subject;
(b) recombining the isolated luminal cells with mesenchymal cells; and
(c) performing a graft in an immunodeficient subject.

34. The method of claim 33, wherein the graft is a renal graft.

35. A method for identifying a compound that inhibits prostate cancer comprising contacting a population of luminal, prostate epithelium stem cells of claim 1 or 2 with a test compound under culture conditions which would cause differentiation of the stem cells into prostate cancer cells, and determining whether the differentiation of prostate cancer cells is inhibited in the presence of the test compound as compared to differentiation of the stem cells in the absence of the test compound.

36. A method for identifying a compound that inhibits prostate cancer, the method comprising:
(a) obtaining a population of luminal, prostate epithelium stem cells of claim 1 or 2;
(b) contacting the population of luminal, prostate epithelium stem cells of claim 1 or 2 with a test compound; and
(c) determining whether the population of luminal, prostate epithelium stem cells of claim 1 or 2 fails to form a tumor in a graft.

37. A method for identifying a compound that inhibits prostate cancer, the method comprising:
(a) obtaining a population of luminal, prostate epithelium stem cells of claim 1 or 2;
(b) contacting the population of luminal, prostate epithelium stem cells of claim 1 or 2 with a test compound; and
(c) determining whether the population of luminal, prostate epithelium stem cells of claim 1 or 2 is reprogrammed to an embryonic differentiation pattern in the presence of the test compound as compared to a population of luminal, prostate epithelium stem cells of claim 1 or 2 that were not treated with the test compound.

38. The population of luminal stem cells of claim 1 or 2, wherein the cells are human stem cells or mouse stem cells.


SEQUENCE LISTING (22 sequences)

SEQ ID NO: 1 - DNA, 31 bp, Artificial Sequence
  Description of Artificial Sequence Synthetic primer
accggaattctccgctgcgcgccgcttttgc

SEQ ID NO: 2 - DNA, 31 bp, Artificial Sequence
  Description of Artificial Sequence Synthetic primer
acccaagcttcatgcctgcaggtcggaggcc

SEQ ID NO: 3 - DNA, 30 bp, Artificial Sequence
  Description of Artificial Sequence Synthetic primer
ctagtctagagcggctcacctccttcctca

SEQ ID NO: 4 - DNA, 32 bp, Artificial Sequence
  Description of Artificial Sequence Synthetic primer
ctagtctagaggatggcaggagaggtcactgc

SEQ ID NO: 5 - DNA, 20 bp, Artificial Sequence
  Description of Artificial Sequence Synthetic primer
ctccgctaccctaagcatcc

SEQ ID NO: 6 - DNA, 23 bp, Artificial Sequence
  Description of Artificial Sequence Synthetic primer
gacactgtcatattacttggacc

SEQ ID NO: 7 - DNA, 19 bp, Artificial Sequence
  Description of Artificial Sequence Synthetic primer
cagatggcgcggcaacacc

SEQ ID NO: 8 - DNA, 21 bp, Artificial Sequence
  Description of Artificial Sequence Synthetic primer
gcgcggtctggcagtaaaaac

SEQ ID NO: 9 - DNA, 24 bp, Artificial Sequence
  Description of Artificial Sequence Synthetic primer
gccaacctgcctcaatcactaagg

SEQ ID NO: 10 - DNA, 24 bp, Artificial Sequence
  Description of Artificial Sequence Synthetic primer
ttccacatacacttcattctcagt

SEQ ID NO: 11 - DNA, 24 bp, Artificial Sequence
  Description of Artificial Sequence Synthetic primer
gccaacctgcctcaatcactaagg

SEQ ID NO: 12 - DNA, 23 bp, Artificial Sequence
  Description of Artificial Sequence Synthetic primer
ccgcgctgtactggaggctgaag

SEQ ID NO: 13 - DNA, 23 bp, Artificial Sequence
  Description of Artificial Sequence Synthetic primer
atactgcaccgggcgggaaggat

SEQ ID NO: 14 - DNA, 19 bp, Artificial Sequence
  Description of Artificial Sequence Synthetic primer
actcaaggcagggatgagc

SEQ ID NO: 15 - DNA, 22 bp, Artificial Sequence
  Description of Artificial Sequence Synthetic primer
gtcatcttcacttagccattgg

SEQ ID NO: 16 - DNA, 21 bp, Artificial Sequence
  Description of Artificial Sequence Synthetic primer
gcgaagagtttgtcctcaacc

SEQ ID NO: 17 - DNA, 21 bp, Artificial Sequence
  Description of Artificial Sequence Synthetic primer
ggagcgggagaaatggatatg

SEQ ID NO: 18 - DNA, 21 bp, Artificial Sequence
  Description of Artificial Sequence Synthetic primer
aaagtcgctctgagttgttat

SEQ ID NO: 19 - protein, 234 aa, Homo sapiens
MLRVPEPRPGEAKAEGAAPPTPSKPLTSFLIQDILRDGAQRQGGRTSSQRQRDPEPEPEPEPEGGRSRAGAQNDQLSTGPRAAPEEAETLAETEPERHLGSYLLDSENTSGALPRLPQTPKQPQKRSRAAFSHTQVIELERKFSHQKYLSAPERAHLAKNLKLTETQVKIWFQNRRYKTKRKQLSSELGDLEKHSSLPALKEEAFSRASLVSVYNSYPYYPYLYCVGSWSPAFW

SEQ ID NO: 20 - DNA, 3281 bp, Homo sapiens
gcggtgcgggccgggcgggtgcattcaggccaaggcggggccgccgggatgctcagggttccggagccgcggcccggggaggcgaaagcggagggggccgcgccgccgaccccgtccaagccgctcacgtccttcctcatccaggacatcctgcgggacggcgcgcagcggcaaggcggccgcacgagcagccagagacagcgcgacccggagccggagccagagccagagccagagggaggacgcagccgcgccggggcgcagaacgaccagctgagcaccgggccccgcgccgcgccggaggaggccgagacgctggcagagaccgagccagaaaggcacttggggtcttatctgttggactctgaaaacacttcaggcgcccttccaaggcttccccaaacccctaagcagccgcagaagcgctcccgagctgccttctcccacactcaggtgatcgagttggagaggaagttcagccatcagaagtacctgtcggcccctgaacgggcccacctggccaagaacctcaagctcacggagacccaagtgaagatatggttccagaacagacgctataagactaagcgaaagcagctctcctcggagctgggagacttggagaagcactcctctttgccggccctgaaagaggaggccttctcccgggcctccctggtctccgtgtataacagctatccttactacccatacctgtactgcgtgggcagctggagcccagctttttggtaatgccagctcaggtgacaaccattatgatcaaaaactgccttccccagggtgtctctatgaaaagcacaaggggccaaggtcagggagcaagaggtgtgcacaccaaagctattggagatttgcgtggaaatctcagattcttcactggtgagacaatgaaacaacagagacagtgaaagttttaatacctaagtcattcctccagtgcatactgtaggtcattttttttgcttctggctacctgtttgaaggggagagagggaaaatcaagtggtattttccagcactttgtatgattttggatgagttgtacacccaaggattctgttctgcaactccatcctcctgtgtcactgaatatcaactctgaaagagcaaacctaacaggagaaaggacaaccaggatgaggatgtcaccaactgaattaaacttaagtccagaagcctcctgttggccttggaatatggccaaggctctctctgtccctgtaaaagagaggggcaaatagagagtctccaagagaacgccctcatgctcagcacatatttgcatgggagggggagatgggtgggaggagatgaaaatatcagcttttcttattcctttttattccttttaaaatggtatgccaacttaagtatttacagggtggcccaaatagaacaagatgcactcgctgtgattttaagacaagctgtataaacagaactccactgcaagagggggggccgggccaggagaatctccgcttgtccaagacaggggcctaaggagggtctccacactgctgctaggggctgttgcatttttttattagtagaaagtggaaaggcctcttctcaacttttttcccttgggctggagaatttagaatcagaagtttcctggagttttcaggctatcatatatactgtatcctgaaaggcaacataattcttccttccctccttttaaaattttgtgttcctttttgcagcaattactcactaaagggcttcattttagtccagatttttagtctggctgcacctaacttatgcctcgcttatttagcccgagatctggtctttttttttttttttttttttttttttccgtctccccaaagctttatctgtcttgactttttaaaaaagtttgggggcagattctgaattggctaaaagacatgcatttttaaaactagcaactcttatttctttcctttaaaaatacatagcattaaatcccaaatcctatttaaagacctgacagcttgagaaggtcactactgcatttataggaccttctggtggttctgctgttacgtttgaagtctgacaatccttgagaatctttgcatgcagaggaggtaagaggtattggattttcacagaggaagaacacagcgcagaatgaagggccaggcttactgagctgtccagtggagggctcatgggtgggacatggaaaagaaggcagcctaggccctggggagcccagtccactgagcaagcaagggactgagtgagccttttgcaggaaaaggctaagaaaaaggaaaaccattctaaaacacaacaagaaactgtccaaatgctttgggaactgtgtttattgcctataatgggtccccaaaatgggtaacctagacttcagagagaatgagcagagagcaaaggagaaatctggctgtccttccattttcattctgttatctcaggtgagctggtagaggggagacattagaaaaaaatgaaacaacaaaacaattactaatgaggtacgctgaggcctgggagtctcttgactccactacttaattccgtttagtgagaaacctttcaattttcttttattagaagggccagcttactgttggtggcaaaattgccaacataagttaatagaaagttggccaatttcaccccattttctgtggtttgggctccacattgcaatgttcaatgccacgtgctgctgacaccgaccggagtactagccagcacaaaaggcagggtagcctgaattgctttctgctctttacatttcttttaaaataagcatttagtgctcagtccctactgagtactctttctctcccctcctctgaatttaattctttcaacttgcaatttgcaaggattacacatttcactgtgatgtatattgtgttgcaaaaaaaaaaaaaaagtgtctttgtttaaaattacttggtttgtgaatccatcttgctttttccccattggaactagtcattaacccatctctgaactggtagaaaaacatctgaagagctagtctatcagcatctgacaggtgaattggatggttctcagaaccatttcacccagacagcctgtttctatcctgtttaataaattagtttgggttctctacatgcataacaaaccctgctccaatctgtcacataaaagtctgtgacttgaagtttagtcagcacccccaccaaactttatttttctatgtgttttttgcaacatatgagtgttttgaaaataaagtacccatgtctttattagattta

SEQ ID NO: 21 - protein, 237 aa, Mus musculus
MLRVAEPREPRVEAGGRSPWAAPPTQSKRLTSFLIQDILRDRAERHGGHSGNPQHSPDPRRDSAPEPDKAGGRGVAPEDPPSIRHSPAETPTEPESDAHFETYLLDCEHNPGDLASAPQVTKQPQKRSRAAFSHTQVIELERKFSHQKYLSAPERAHLAKNLKLTETQVKIWFQNRRYKTKRKQLSEDLGVLEKNSPLSLPALKDDSLPSTSLVSVYTSYPYYPYLYCLGSWHPSFW

SEQ ID NO: 22 - DNA, 3137 bp, Mus musculus
cagcggaggagggatgcttagggtagcggagccccgagagccacgggtggaggcgggtggccgcagtccttgggcagcgccacccacgcagtccaagcggctcacctccttcctcatccaggacatcctgcgggaccgcgcggagcggcacgggggacactcaggcaatccgcagcactcgccggaccctaggagggactccgctccagagcccgacaaagcagggggtcgcggcgtggctccggaggacccaccaagtatccggcatagccccgcggagacaccgactgaacccgagtctgatgcacattttgagacttatcttttggactgtgaacataatccaggggacttagcaagtgccccccaggtcaccaagcagccacagaagcgctcccgggccgccttctctcacactcaggtgattgagttggagaggaagttcagccatcagaagtacctgtctgcccctgagagggctcacctggccaagaacctcaaactcaccgaaacccaagtcaaaatatggttccagaacagacgctataagaccaagcgaaagcagctgtcggaagacctgggagtcttggagaagaactcaccattgtctttgccagccctgaaagatgacagcctgcccagtacctccttggtctccgtgtatactagctatccctactacccctacctgtactgtctgggcagctggcatccatctttctggtagcaacagttctgctgataactgcatatatcagaaactgtcttccctgggggtctctgggaaaaagccacagtggctgatgtcaaggagtcggaagcaagaaatgtacagatgcaaactgttgggggttcccagggaacactccaattcttctctggtgggcaggtgaaagatctggggcagtgactatttgaagatgtagtcccagttccaagtgtcctgtaggtgactgctgcccgctcccctgtttagagagagccaggcaagtttgagtcttgtttccagaacttagaatggctacagatgcaatggctatatcgttcctaggaattctgttgtggcaactccatcctctcccatgactgagtatcatggaagggccaagcaaggggagggatggccccttagatgaggatttcctcttgagttaaacttgggtctcctgttggccttggactctggccagagtttctctctgcccttgtaaaaatgagggtaaacggaagatttgaagaatgggggtgggagggggtgacccttagtgattgaggcaggttggcatagaacgtgaagggggtggaaggtggaattttcaacttacccttttcacagtgatgtgccagtccaaggatttacagggcagcctgagtgtggcagggtacacacgcggagggggttaagacaagctgtgtaaacagtttgcctgcaggggtgggctgcaaaacagacttcctctccaagacatgggcctatggggtttccacacgccaggtgagcggcttccatcaggactttatattagcagggaagaaaaggactctcctgcccttcctgcttagactcaggagggcctgcagcttccctggcctgaaaattcacactttgtgatgacagaggtgccattagttttaaacaaaggaccttccaaccctcctttttatttcttgttctttcctacaggacctctccgttcatgcttttcatctgtgcctgtcttatttcgtgtaagatttgctcccacctcctcccaaggtttacctttcttggtttaacaacttaactagctacagacagattatgacatacttttaaaagaaaaaaatgagcaactcatctcttccctttaaaaatatgacatactttagagagagaaaaaaaacctagcaactcctctctctctttcctttaaaaatactcagcattaaatcccaagccctatttaaagacatgagagctgaagatgacaggttctttcgcagggctgtgtgctagctctgtgttacagttcaaaggctgatgcccattggggtcctggggatccctgtaggcagaggtggtttaggggtgcttgtttttcatgtgtaaggaagaacaggacaaaaggaagggctggcactgctgagggatctgggggagatggaggggtcctgctgtcagcccaagctaaccagcaaggagaggtaaactgtcttcaaaatacaacctcaccgaggcttccatccacagatgtgtgtgtgtgtgtgtgcgcgcgcgcatgcacgcgcaaatgctttggagaactatgcttaggaactaccctaaagtggtaggagtgaaaaagagaattggttgaccttcccttttccatcctgatatctcaggtaacctgccgaatgggagataaaggaggtagagcacacttataacaaatgagactttctggggccaggaaatccctttatcccaccactcagtgctatacagtgagaaatcttttaatttcctccttttaaatgggccaatttactgttggtggcaaaactgccaaaggaagttgatagaaagctggcggcttgcaccctgttctttgacagcccgggtccccatgttaaagtattgttccgtgtcccttgttgctgaccactgtggtagcgttagaccataccaaagccggaggagtccagttctcttctttctatccgtcttttggctctgagttccctatcagtactttctctcctctcactctgactagaatctttttcagcgtgcactttcactacgtttgaactattatagtgtgcagtgtgtggcaaagtcagaaagggaaaggcttgtgtttaaaattatgttggggaagtctgactcaattttgctttctccatactggaactacccattcacccacacctgaactggtagaaacgtggcctgttgtctccagcctgtgaatgggaaaggtatcgctgtgggtatcacctgggtgacatatctctgtcctgtttcgtcaatcagttagcaaaccccggctctaacagaaaagtctgtcacttggcatttagtatagtactccactcaaaactttatttttctacgggttttttttttttttgcaacctatgagtgttttaaaataaagcctctatgtctttat